(19) United States
(12) Patent Application Publication
(10) Pub. No.: US 2025/0340587 A1
(43) Pub. Date: Nov. 6, 2025

(54) INSOLUBLE SUPPORT FOR SOLID PHASE SYNTHESIS

(71) Applicant: PolyPeptide Laboratories Holding (PPL) AB, Limhamn (SE)

(72) Inventors: John Lee, Carmel, IN (US); Byoung J. Min, Rolling Hills Estates, CA (US); Ayat Mohsin Garawi, Cerritos, CA (US); Hanich Hossein Nejad Ariani, Los Angeles, CA (US); Tharwat Mohy-Eldine, Strasbourg (FR); Olivier Ludemann-Hombourger, Rohrwiller (FR)

(21) Appl. No.: 18/869,999

(22) PCT Filed: May 31, 2023
(86) PCT No.: PCT/SE2023/050541

§ 371 Date: Nov. 27, 2024

Related U.S. Application Data
(60) Provisional application No. 63/347,168, filed on May 31, 2022.

(30) Foreign Application Priority Data
May 31, 2022 (SE) 2230168-3

(51) Int. Cl. C07K 1/04; C08F 12/36

(52) U.S. Cl. CPC C07K 1/042; C08F 12/36

(57) ABSTRACT

The present invention relates to an insoluble support comprising distal binding sites, the support comprising a homogeneous polymeric matrix and constructs, the constructs covalently bound to the polymeric matrix, wherein the constructs comprise at least one branching agent selected from aminoalkanoic acids comprising at least 2 amino groups and from 3 up to 10 carbon atoms, cleavable linkers and at least one spacer coupled to at least one branching agent via an amide bond, the cleavable linkers providing the distal binding sites.

### FIELD OF THE INVENTION

The present invention relates to insoluble supports comprising constructs for use in solid phase organic synthesis such as solid phase morpholino oligomer synthesis, solid phase oligonucleotide synthesis and solid phase peptide synthesis protocols, methods for the preparation of insoluble supports and methods for the synthesis of polypeptides using the insoluble support.

### BACKGROUND AND OBJECTS OF THE INVENTION

Heterogeneous liquid-solid phase chemical reaction protocols are attractive for the synthesis of molecules comprising recurrent sub-units. The liquid-solid phase protocol enables the effective separation of the solid phase from the liquid phase providing suitable conditions for the application of recurrent cycles comprising reaction steps for the successive, step-wise introduction (addition) of sub-units. Liquid-solid phase reaction protocols have been successfully implemented in the field of peptide, morpholino oligomer and oligonucleotide synthesis. Under the course of the synthesis the nascent molecule (such as a growing peptide, morpholino oligomer or oligonucleotide) is covalently bound to the insoluble support providing the conditions for the efficient removal of by-products by washing between reaction steps of the recurrent cycles.

Solid phase peptide, morpholine and nucleotide synthesis is an established methodology to produce peptides. The peptide, is synthesized in a single reaction vessel thereby reducing process complexity. Further, isolation and purification of intermediates are avoided or significantly reduced. The possibility to use excess reactants translates into commercially relevant yields with commercially attractive purity already prior to post synthesis purification.

The peptide, morpholino oligomer or oligonucleotide is gradually synthesized on an insoluble support (solid phase). The insoluble support provides binding sites for the peptide to be synthesized. Whereas solid phase peptide synthesis provides significant advantages as elaborated herein, the reactions occur on an insoluble support (phase) while crucial reactants are present in the liquid phase. Hence, solid phase synthesis by its very nature creates a phase boundary which needs to be negotiated. The interaction of the solid and liquid phase is important whereby the swelling and solvation of the solid phase are important properties. Swelling and solvation influence e.g. diffusion and accessibility of the reagents into the solid phase and the consumption of washing solution.

A further property of the solid phase is the number of binding sites available for peptide synthesis, often denoted as loading. An increased number of binding sites may not automatically generate an increase in the capacity of the insoluble support and purity of the peptide. An increase of the capacity of the insoluble support translates into an increased yield (in terms of peptide output). However, binding sites must be made available for peptide synthesis and high loading is known to be detrimental to purity and yield of the synthetized peptide especially when the number of amino acids increase.

Non-adequate swelling and solvation of the solid phase often translates into poor reaction site accessibility and diminished reaction rates. Additionally, swelling and solvation is influenced during peptide synthesis as the solid phase is gradually modified by the coupling of amino acids to the nascent peptides bound to the solid phase.

For synthesizing a target polypeptide, one chooses a combination of solid support and liquid phase for optimizing yield and crude target polypeptide purity. Usually, such an optimization translates into a synthesis specification striving to increase swelling and solvation of the solid phase as much as possible. Increased swelling also equates to increasing volume of the reaction composition.

Sometimes the reactor volume of a solid phase peptide synthesis process may be a limiting factor. The present invention provides a novel insoluble support which is obtained by converting standard resins with a construct comprising readily available amino acids, said insoluble supports significantly increasing reactor throughput while exhibiting good crude peptide yield and commercially relevant purities. Thus, by applying the novel insoluble support in a given reactor the reactor throughput can be significantly increased. The novel insoluble support offers the opportunity to increase the output of a polypeptide without increasing the reactor volume.

For shorter polypeptides, i.e. polypeptides with a limited number of amino acids, e.g. under 10 amino acids, an increase of the binding sites of the resin (often referred to as loading [mmol binding sites/g resin]) usually translates into increased yield while still attaining a satisfactory purity. As the number of amino acids of the polypeptide increases an increase of the loading does not necessarily translate into higher yield and useful purity. Quite the opposite, a polypeptide with a significant number of amino acids (e.g. above 30 amino acids) may provide poor yield or not even be possible to synthesis if not the loading of a resin is reduced. Thus, when synthesizing a long polypeptide usually a resin with a low loading is selected.

With the insoluble supports of the invention, it is possible to synthesize complex, long polypeptides at high yield and commercially useful purity.

Lee et al (Tetrahedron Letters 41 (2000) 7481-7485) discloses resins with a cross-linked polystyrene (PS) core and a poly(ethylene glycol) (PEG) shell prepared by suspension polymerization for use in solid-phase synthesis. The PEG monomer used in the suspension polymerization is formed by reacting O,O′-Bis(2-aminopropyl) polyethylene glycol 500 and methacryloyl chloride thereby providing a PEG comprising an ammonium function. The provision of a charged PEG macromonomer under the polymerization conditions outlined in Lee provides polymeric beads with a core of cross-linked polystyrene and the PEG macromonomers arranged at the surface of the bead thereby providing a PEG shell. The PS core is highly cross-linked with over 4% divinylbenzene (DVB). The loading (substitution) of the core-shell resin may be increased by modifying the shell with coupling of lysine. The presence of the PEG shell covering the core together with the high degree of cross-linking of the core prevents polypeptide synthesis within the core. All synthesis happens at the shell.

Lee et al (Tetrahedron Letters 42 (2001) 7443-7445) discloses a tris(hydroxymethyl)aminomethane (Tris) based dendrimer monomer used for increasing the loading capacity of a core-shell type resin as disclosed in Lee (2000) and a gel type TentaGel resin. Lee 2001 fails to teach monomers based on aminoalkanoic acids, such as diaminoalkanoic acids for use as the repeating unit for dendrimerization of a support. Also, Lee (2001) does not disclose the use of spacer molecules between the support and dendrimer monomer or between dendrimer monomers.

Chan et al (Tetrahedron Letters 40 (1999) 4909-4912) discloses the synthesis of tri-amino acids and the use of such molecules for the generation of dendrimers on resins thereby increasing the loading of the resin. Chan et al fails to disclose the application of spacer molecules between the PS resin or between the generations of tri-amino acid units.

The present invention provides an insoluble support increasing the yield and specifically the reactor throughput (mass of product after cleavage per swelling of insoluble support) of the product (polypeptide) at a commercially acceptable purity at a given reaction volume. Thus, a homogeneous polymeric matrix for e.g. solid phase peptide synthesis modified according to the present invention, i.e. by the provision of a construct of at least one branching agent, thereby providing a modified polymeric matrix, an insoluble support, significantly increases the yield and the reactor throughput of the product (polypeptide) at a commercially acceptable purity at a given reaction volume. The insoluble support of the invention also enables the synthesis of long (complex) polypeptides such as polypeptides having more than e.g. 25 amino acids in good yield and purity.

An objective of the present invention is to increase the throughput (mass of crude peptide after cleavage as a function of the volume of the resin with the target polypeptide [mass/volume g/L]) of a target polypeptide in a given solid phase peptide synthesis reactor.

A further objective is the provision of an insoluble support (polymeric insoluble support) capable of increasing capacity/yield while at the same time reducing the disproportional increase of the swelling of the insoluble support without impacting the purity of the synthetized peptide.

A further objective is the provision of an insoluble support obtained by modifying a base resin thereby increasing capacity/yield for a given reactor volume and simultaneously reduce the solvent consumption, in absolute terms and/or solvent consumption per unit of synthesized target polypeptide specifically when compared to the non-modified base resin.

A further objective is the provision of an insoluble support reducing the solvent consumption per unit of peptide (e.g. per mmol of peptide), preferably compared to the un-modified insoluble support.

A still further objective is the provision of an insoluble support which reduces the swelling and/or which reduces the solvent consumption (per synthesized unit of target peptide) while at least maintaining, or significantly increasing, the capacity/yield compared to the un-modified insoluble support.

A further objective is the provision of an insoluble support (modified resin) which significantly increases the yield of (crude) peptide without significantly reducing purity at a given reactor volume (compared to the non-modified resin).

A further objective is the provision of an insoluble support for the synthesis of long polypeptides at good yield and purity where the insoluble support is formed from commercially available resins and modified using SPPS and readily available compounds such as amino acids.

A further objective is the provision of an insoluble support (modified resin) for increasing the throughput (mass of crude peptide after cleavage as a function of the volume of the resin with the target polypeptide [mass/volume g/L]) compared to the non-modified resin while preserving a commercially relevant purity of the (crude) target polypeptide.

A still further objective is to increase the throughput (mass of crude peptide after cleavage as a function of the volume of the resin with the target polypeptide) of an insoluble support (modified resin) compared to the non-modified polymeric matrix/resin while preserving a commercially relevant purity of the (crude) target polypeptide while also reducing the final volume of the polymeric matrix/resin.

As alluded to above, swelling is an important property of an insoluble support implemented in peptide synthesis. Generally, an increase in swelling facilitates the diffusion of reagents (e.g. amino acids) into the insoluble support potentially also increasing the availability of binding sites. However, increased swelling usually increases the required reactor volume and solvent consumption. The present invention provides for an insoluble support (modified insoluble support) which increases throughput at a given reactor volume and reduces solvent consumption while increasing capacity/yield and throughput (cleaved target peptide as function of volume of final swelling of insoluble support prior to cleavage) compared to the un-modified polymeric matrix.

The implementation of an insoluble support in accordance with the invention is an efficient measure for increasing the capacity of a given reactor volume. Furthermore, the implementation of the insoluble support increases the capacity of a solid phase peptide synthesis without increasing the reaction volume (volume of the reaction vessel) or even decreasing the reaction volume while also reducing solvent consumption while maintaining the same conventional polymeric matrix.

The insoluble support is preferably used as the solid phase in solid phase peptide synthesis (SPPS), such as an Fmoc/tBu or Boc SPPS strategy. The implementation of the insoluble support in SPPS significantly increases the throughput of the target peptide in relation to the non-modified base resin/polymeric matrix.

The insoluble support of the invention may also be successfully applied in aqueous-based solid phase peptide synthesis protocols, i.e. where the solvent used is aqueous-based. Thus, a further objective of the present invention is to provide SPPS conditions enabling the reduction of organic solvents and/or replacing organic solvents with aqueous-based solvents.

### SUMMARY OF THE INVENTION

One embodiment of the invention relates to an insoluble support comprising constructs increasing the number of available binding sites compared to the non-modified polymeric matrix. The insoluble supports of the invention are successfully implemented in solid phase synthesis protocols notably in solid phase peptide synthesis. Additionally, the invention also relates to methods for preparation of insoluble supports by modifying polymeric matrices, and methods for synthesizing peptides using the insoluble support or construct.

More specifically, one embodiment relates to an insoluble support (resin) in particulate form comprising distal binding sites, the support comprising a homogeneous polymeric matrix and constructs, the constructs covalently bound to the polymeric matrix, wherein the constructs comprise at least one branching agent selected from aminoalkanoic acids comprising at least 2 amino groups and from 3 up to 10 carbon atoms, cleavable linkers and at least one spacer coupled to at least one branching agent via an amide bond, the cleavable linkers providing the distal binding sites.

A further embodiment relates to a method for forming an insoluble support in particulate form comprising secondary binding sites, the insoluble support comprising a homogeneous polymeric matrix and constructs, the constructs comprising cleavable linkers and at least one spacer, the method comprising providing a polymeric matrix in particulate form comprising primary binding sites, wherein the construct is formed by a solid phase synthesis protocol, the solid phase protocol comprising at least one reaction steps where a branching agent selected from amino protected aminoalkanoic acids comprising at least 2 amino groups and from 3 up to 10 carbon atoms is coupled by an amide bond to either of the primary binding sites of the polymeric matrix, a spacer or a branching agent, the protocol further comprising a reaction step where cleavable linkers are coupled to branching agents either directly or through one or more spacers.

A further embodiment relates to a method for synthesizing a polypeptide, a morpholino oligomer or a oligonucleotide using a solid phase peptide synthesis protocol comprising the application of the insoluble support disclosed herein.

### Definitions

Polymeric matrix: The insoluble support is formed from a polymeric matrix which is modified by a construct covalently attached to the polymeric matrix. The polymeric matrix typically comprises binding sites, also referred to as primary binding sited. The constructs are covalently bound to the biding sites of the polymeric matrix. The polymeric matrix may be modified to comprise any type of binding sites suitable for the coupling the constructs. It should be noted that the primary binding sites of a polymeric matrix are consumed during the formation of the insoluble support of the invention. Thus, the primary binding sites of the polymeric matrix are used to covalently bind the constructs. Ideally, all primary binding sites of the polymeric matrix are consumed by the covalent coupling of constructs signifying that the insoluble support of the invention does not have a polymeric matrix with primary binding sites. The polymeric matrix is typically a polymer network usually comprising polymers cross-linked to a degree providing structural integrity of the polymeric matrix particles while enabling a significant solvation of the polymer network. The homogenous polymeric matrix is preferably selected from polymeric matrices comprising binding sites distributed throughout the polymeric matrix. Typically, the binding sites of the polymeric matrix are distributed essentially homogeneously through (over) the matrix. The polymeric matrix is preferably selected from homogeneous polymeric matrices. The polymeric matrix is typically in particulate form. The polymeric matrix is preferably insoluble. The size of the particles may range from about 1 μm up to about 2000 μm and typically from about 20 μm up to about 500 μm. The polymeric matrix may be formed by emulsion polymerization. Particularly useful homogeneous polymeric matrices are styrene-based matrices cross-linked with a cross-linker suitably divinyl benzene (DVB). Styrene-based homogeneous polymeric matrices are typically cross-linked with a content of cross-linker below 4.0 wt %, preferably below 3.5 wt %, preferably below about 3.0 wt %, suitably from about 0.5 up to 2.5 wt %.

Insoluble support: The insoluble support comprises a polymeric matrix and constructs and constitutes the solid phase in any liquid-solid synthesis protocols such as solid phase peptide synthesis.

Construct: A construct is a molecule increasing the number of binding sites (primary binding sites) of a polymeric matrix. The construct is a branched molecule which may be characterized as a dendrimer or dendron. According to an aspect, the construct may be referred to as a branched polypeptide. A construct per se may also function as an anchor molecule in liquid phase peptide synthesis (LPPS). The constructs comprise cleavable linkers, at least one branching agent and at least one spacer. According to an aspect, the constructs consist of cleavable linkers, at least one branching agent and at least one spacer, that is the construct does only contain compounds selected from cleavable linkers, branching agents and spacers. According to a further aspect, the cleavable linkers, at least one branching agent and at least one spacer are all coupled to each other via amide bonds. The simplest construct comprises one branching agent. Constructs are preferably formed by recurrent coupling branching agents to previous generations of branching agents (repeated branching cycles) thereby obtaining highly ordered, branched macromolecules with ever increasing binding sites as a function of the number of branching agents of a construct. A construct can be formed by a divergent reaction scheme, a convergent reaction scheme or a combination of divergent and convergent reaction schemes. Preferably, the insoluble construct, i.e. the construct is formed by a divergent reaction scheme such as a solid phase organic synthesis scheme and preferably a solid phase peptide synthesis scheme where all compounds making up the construct, inter alia cleavable linker, branching agent, spacer have functional groups enabling that the compounds are coupled to each other via amide bonds.

Branching agent: The branching agent is the molecule responsible for creating the specific structure of the construct and the ability of the construct to increase the number of binding sites of a polymeric matrix. The branching agent comprises at least three binding sites. In its simplest form, a construct comprises only one branching agent, at least one spacer coupled to the branching agent via an amide bond and cleavable linkers coupled to the branching agent.

Distal branching agent: A distal branching agent is a branching agent furthest away from the polymeric matrix. Secondary or distal binding sites are provided by distal branching agents by cleavable linkers coupled to available binding sites of distal branching agents.

Proximal branching agent: A proximal branching agent is the branching agent of a construct bound to the polymeric matrix.

If a construct only has one branching agent, said one benching agent may be designated proximal or distal branching agent.

Intermediate branching agent is a branching agent positioned between a distal and a proximal branching agent. Intermediate branching agents necessitate at least three generations (layers) of branching agents.

Primary, secondary, tertiary, etc branching agent: Primary, secondary branching agents, etc. denote branching agents with respect to other branching agents of the same construct. A primary branching agent or first generation branching agent (first layer branching agent) is the branching agent coupled to the base matrix without any other branching agent between. A secondary branching agent or second generation branching agent (second layer branching agent) is coupled to a primary branching agent. A tertiary branching agent or third generation branching agent (third layer branching agent) is coupled to a secondary branching agent. Put differently, a tertiary branching agent is coupled to the polymeric matrix by way of two other branching agents, a secondary and the primary branching agent. Primary, secondary, tertiary, etc branching agents may also be referred to as primary (first), secondary layers or generations of branching agents.

Distal branching agents are the branching agents of a construct to which the cleavable linkers are attached. The generation comprising the distal branching agent is the generation having the highest integer.

The proximal branching agent is bound to the polymeric matrix with or without one or more spacers. The proximal branching agent is the first-generation branching agent.

The presence of intermediate (generation[s]) of branching agents necessitates at least three generations of branching agents. Intermediate generations are found between distal branching agents and the proximal branching agent.

Coupling: When referring to that a molecule (e.g. branching agents, cleavable linkers and spacers) is coupled to another moiety (e.g. branching agent, spacer or polymeric matrix) such coupling can be a direct coupling or a coupling of the molecule to the other moiety by any number and types of intermediate molecules, herein referred to as spacers or spacer molecules. By coupling herein is usually meant covalent coupling. The terms ‘bound’ and ‘coupling’ are used interchangeably.

Spacer: A spacer, or spacer molecule, is a molecule which does not form part of the definition of the branching agent. A spacer typically comprises (only) two binding sites. Hence, a spacer cannot provide a branching point. While the binding sites of a spacer may be selected from a variety of functional groups, the binding sites of a spacer are preferably chosen such that an amide bond is formed when a spacer is coupled to a branching agent.

Binding site: A binding site (functional group or reactive site) is a site of a molecule or a polymeric matrix, specifically branching agent, spacer, cleavable linker, available for chemical reaction with a binding site of a molecule of another identity. A binding site may be regarded as a functional group with the ability of forming a covalent bond with a molecule different from the molecule comprising a binding site, e.g. a branching agent. Two binding sites usually form a covalent bond.

Primary (1^{st}) binding sites: A primary binding site is a binding site available on the polymeric matrix available for covalently coupling of constructs for the provision of the insoluble supports of the invention suitable for solid phase peptide synthesis.

Secondary (2^{nd}) or distal binding sites: A secondary or distal binding site is a site provided by the construct and more specifically by cleavable linkers covalently bound to the binding sites of distal branching agents optionally with one or more spacers between the linker and binding site of the distal branching agent. A secondary or distal binding site provides the conditions for covalently anchoring peptides during solid phase peptide synthesis.

Cleavable linker. A cleavable linker is a molecule to which a growing polypeptide is covalently coupled further facilitating or making possible the cleavage of peptides from the construct. The construct comprises cleavable linkers which are covalently attached to the binding sites of distal branching agents either directly or through one or more spacers. Preferably, the cleavable linkers are coupled to the branching agents or a spacer via amide bonds.

Wherever mentioning amino acids any relevant amino acid derivatives are also encompassed.

### DISCLOSURE OF THE INVENTION

One embodiment of the invention relates to an insoluble support. A further embodiment relates to a method for forming an insoluble support. An additional embodiment pertains to a solid phase peptide synthesis protocol for synthesizing polypeptides, morpholino oligomers or oligonucleotides comprising the application of the insoluble support.

The insoluble support preferably functions as the solid phase in solid phase peptide synthesis. According to an aspect, the insoluble support is used as the solid phase in solid phase chemical synthesis protocols. In particular, the insoluble support is used as the solid phase in solid phase morpholino oligomer synthesis, solid phas oligonucleotides synthesis and solid phase peptide synthesis. More particularly, the insoluble support comprises binding sites, herein also referred to as secondary binding sites or distal binding sites and a polymeric matrix where the distal binding sites are provided by constructs, the constructs comprising at least one branching agent, cleavable linkers and at least one spacer. A branching agent is positioned between the secondary binding sites and the polymeric matrix. The polymeric matrix is preferably in particulate form representing a solid phase.

The polymeric matrix typically comprises binding sites, herein also referred to as primary binding sites. The polymeric matrix preferably comprises binding sites suited for coupling of constructs or functioning as the binding sites (anchoring points) for the synthesis of the constructs. The type of polymeric matrix is dependent on the solid phase synthesis application. The polymeric matrix may comprise polymers such as comprising a polymeric network, e.g. a crosslinked polymeric network. Typically, the polymeric matrix is based on commercially available polymeric matrices (resins) configured for the use as the solid phase in peptide or nucleotide synthesis. The construct is the entity increasing the number of binding sites of a polymeric matrix such that one primary binding site (of the polymeric matrix) provides for several secondary binding sites (at least two) by way of the construct covalently bound to the primary binding site. A construct being covalently bound to the polymeric matrix comprises at least a branching agent, said branching agent being directly bound to a binding site of the polymeric matrix or covalently bound to the primary binding site by any number and any type of molecules (also referred to as pacers or spacer molecules) between branching agent and primary binding site of the polymeric matrix.

The construct may be denoted as a branched molecule. According to an aspect, the construct may be referred to as a branched molecule comprising amino acids, or as a branched peptide/polypeptide. The construct comprises at least one branching agent, cleavable linkers, and at least one spacer. A branching agent is a molecule comprising at least three binding sites. A spacer is a molecule which cannot function as a branching agent. Thus, a spacer is a molecule preferably having two binding sites or preferably having exactly two binding sites.

The insoluble support of the present invention is herein defined (claimed) in several ways (aspects) to adequately frame the invention. However, all aspects of the insoluble support relate to the same basic concept, the provision of an insoluble support comprising constructs, the construct comprising at least one branching agent, cleavable linkers and at least one spacer, increasing the number of binding sites and typically providing a specific geometrical configuration.

Some aspects of the insoluble support are defined by the characterization of the final insoluble support. Some aspects of the insoluble support are partly defined by introducing features relating more to how the insoluble support is formed.

An example of a simple, non-complex construct comprises only one branching agent, such as one diaminoalkanoic acid, e.g. lysin, ornithine, etc, suitable cleavable linkers coupled to the branching agent thereby providing secondary (distal) binding sites, and e.g. a glycine as a spacer, the spacer coupled to the branching agent via an amide bind and a binding site of the polymeric matrix.

One aspect of the invention relates to an insoluble support in particulate form for use in solid phase peptide synthesis, solid phase morpholino oligomer synthesis, solid phase oligonucleotides synthesis, preferably for use in solid phase peptide synthesis, the insoluble support providing binding sites (distal or secondary binding sites), the insoluble support comprising a homogeneous polymeric matrix and constructs, the constructs covalently bound to the polymeric matrix, the constructs comprising at least one branching agent, cleavable linkers and at least one spacer, the at least one branching agent selected from aminoalkanoic acids comprising at least 2 amino groups and from 3 up to 10 carbon atoms, the at least one spacer coupled to at least one branching agent by an amide bond, the cleavable linkers providing the distal binding sites and being covalently bound to the at least one branching agent.

According to a further aspect, the insoluble support may be obtainable by the provision of a homogeneous polymeric matrix comprising primary binding sites and by the application of recurrent synthesis steps comprising the coupling of branching agents such as aminoalkanoic acids comprising at least 2 amino groups and from 3 up to 10 carbon atoms and at least one spacer, and eventually coupling cleavable linkers to binding sites of the distal branching agent (or distal branching agents) thereby forming a construct said construct providing at least two secondary/distal binding sites.

According to an embodiment the branching agent is selected from aminoalkanoic acids comprising at least 2 but not more than 3 amino groups and from 3 up to 10 carbon atoms, suitably from 3 up to 8 carbon atoms, suitably from 4 up to 8 carbon atoms.

According to a further embodiment the branching agent is selected from diaminoalkanoic acids comprising from 3 up to 10 carbon atoms, suitably from 3 up to 8 carbon atoms, suitably from 4 up to 8 carbon atoms.

The number of carbon atoms of any of the aminoalkanoic acids disclosed herein may range from 3, from 4, from 5, from 6 and may range up to 10, up to 9, up to 8, up to 7, and any range of combination of lower and upper number.

According to a further embodiment the branching agent is selected from 2,3-diaminopropionic acid (Dpr), 2,4-diaminobutyric acid and 2,5-diaminopentanoic acid (ornithine), 2,6-diaminohexanoic acid, suitably the branching agent is selected from 2,4-diaminobutyric acid and 2,5-diaminopentanoic acid (ornithine), 2,6-diaminohexanoic acid.

According to an embodiment all branching agents of a construct are selected from aminoalkanoic acids comprising at least 2 amino groups and from 3 up to 10 carbon atoms.

According to an embodiment all branching agents of a construct are identical and selected from aminoalkanoic acids comprising at least 2 amino groups and from 3 up to 10 carbon atoms.

According to an embodiment the branching agents of a construct may be any combination of branching agents selected from aminoalkanoic acids comprising at least 2 amino groups and from 3 up to 10 carbon atoms.

One embodiment relates to an insoluble support, where the number of branching agents δ of a construct is given by the formula δ(n)=2ⁿ−1, where n denotes number of generations of branching agents and n is a positive integer.

One embodiment relates to an insoluble support, where the number of distal binding site λ of a construct is given by the formula λ(n)=2″, where n denotes number of generations of branching agents and n is a positive integer.

One embodiment relates to an insoluble support, where the number of branching agents δ of a construct is given by the formula δ(n)=2ⁿ−1 and the number of distal binding site λ of a construct is given by the formula λ(n)=2″, where n denotes number of generations of branching agents and n is a positive integer.

According to one embodiment the number of generations n of branching agents of a construct is preferably from 1 to 10, suitably from 1 to 5.

According to a further embodiment the number of generations n of branching agents of a construct is preferably from 2 to 10, suitably from 2 to 5.

The construct comprises at least one spacer. If the construct contains only one branching agent at least one spacer may be positioned between the branching agent and the polymeric matrix. Alternatively, a spacer may be positioned between each of the two or more cleavable linkers and the branching agent.

If the construct comprises multiple generations of branching agents, i.e. two or more generations, spacers are typically positioned between any one of the following positions: between the branching agents of any generation, between cleavable linkers and (distal) branching agents, between (proximal) branching agent and the polymeric matrix. Any number of spacers may be present at any of the positions.

According to an aspect at least one spacer is positioned between branching agents. Spacers between branching agents signify that the construct comprises at least two generations of branching agents. An example is a construct comprising three lysine branching agents, three glycine spacers and four cleavable linkers. Two glycine spacers are positioned between the first generation lysine and the two second generation lysines. More specifically, one glycine spacer each is covalently attached to the epsilon and alpha amines of the first generation lysine via amide bonds. Furthermore, two second generation lysines are covalently attached to each of the two glycine spacers via amide bonds. Additionally, a further third glycine spacer may be positioned between the first generation lysine and the homogeneous polymeric matrix by way of two amide bonds. Four cleavable linkers are coupled to the epsilon and alpha amines of the two second generation lysines via fours amide bonds.

According to an aspect multiple spacers are positioned between branching agents. If three or more spacers are positioned between branching agents, between branching agent and polymeric matrix, and/or between cleavable linkers and branching agents, at least one spacer is coupled to two spacers, preferably by two amide bonds.

According to an embodiment, the spacer or spacers are coupled to a branching agent via an amide bond.

The modification of a polymeric matrix with branching agents such as an aminoalkanoic acids comprising at least 2 amino groups and from 3 up to 10 carbon atoms converts one binding site (primary binding site) of the polymeric matrix into two or more binding sites (secondary or distal binding sites).

Distal binding sites are binding sites provided after modification of a polymeric matrix.

Distal binding sites are binding sites for synthesis in solid phase synthesis such as solid phase peptide synthesis.

The number of additional secondary binding sites is correlated with the number of branching agents comprised in the construct. More importantly, the increase of secondary binding sites is preferably achieved by coupling branching agents to other branching agents consecutively of which one branching agent, the proximal branching agent, is coupled to the polymeric matrix, optionally with spacers, thereby generating layers or generations of branching agents. Such layers may be referred to as primary (first), secondary (second), tertiary (third), quaternary (fourth), etc. layers or generations. In aspects of the invention branching agents are denoted primary, secondary, tertiary, quaternary branching agents. For example, tertiary branching agents form the tertiary (third) layer or third generation.

One aspect of the invention relates to an insoluble support in particulate form comprising a homogeneous polymeric matrix and constructs, the constructs covalently bound to the polymeric matrix, the constructs comprising at least one branching agent, cleavable linkers and at least one spacer; the branching agent selected from aminoalkanoic acids comprising at least 2 amino groups and from 3 up to 10 carbon atoms, of which at least one branching agent is a distal branching agent comprising binding sites, the cleavable linkers covalently bound to the binding sites of the distal branching agent either directly or by way of one or more spacers, each cleavable linker providing a distal binding site; the at least one spacer positioned at anyone of the following locations: between branching agents, between polymeric matrix and branching agent, between binding sites of the distal branching agents and cleavable linkers.

One aspect of the invention relates to an insoluble support in particulate form comprising a polymeric matrix and constructs, the constructs covalently bound to the polymeric matrix, the constructs comprising at least two branching agents, cleavable linkers and spacers, one branching agent denoted distal branching agent and one branching agent denoted proximal branching agent and optional branching agents denoted intermediate branching agents; the branching agents selected from diaminoalkanoic acids comprising from 3 up to 10 carbon atoms, the distal branching agent comprising binding sites, the cleavable linkers covalently bound to the binding sites of the distal branching agent either directly or by way of one or more spacers, each cleavable linker providing a distal binding site; the spacers positioned at anyone of the following locations: between branching agents, between polymeric matrix and branching agents, between binding sites of the distal branching agents and linkers, and the distal branching agent bound to only one branching agent selected from either optional intermediate branching agents or the proximal branching agent; the optional intermediate branching agents bound to three branching agents selected from distal branching agents, optional intermediate branching agents and a proximal branching agent, and the proximal branching agent bound to two branching agents selected form distal branching agents and optional intermediated branching agents.

One aspect of the invention relates to an insoluble support in particulate form comprising a polymeric matrix and constructs, the constructs covalently bound to the polymeric matrix, the constructs comprising at least three branching agents, cleavable linkers and spacers; the branching agents denoted distal branching agents, intermediate branching agents and proximal branching agent; the branching agents having three binding sites, the distal branching agents selected from diaminoalkanoic acids comprising from 3 up to 10 carbon atoms, the cleavable linkers covalently bound to the binding sites of the distal branching agent either directly or by way of one or more spacers, each cleavable linker providing a distal binding site; the spacers positioned at anyone of the following locations: between branching agents, between polymeric matrix and branching agents, between binding sites of the distal branching agents and linkers, and the distal branching agent bound to only one intermediate branching agent; the intermediate branching agent bound to three branching agents selected from distal branching agents, intermediated branching agents and a proximal branching agent, and the proximal branching agent bound to two intermediate branching agents.

One aspect of the invention relates to an insoluble support in particulate form comprising a polymeric matrix and constructs, the constructs covalently bound to the polymeric matrix, the constructs comprising one branching agent, two cleavable linkers and at least one spacer; the branching agent selected from selected from diaminoalkanoic acids comprising from 3 up to 10 carbon atoms comprising three binding sites, the two cleavable linkers covalently bound to the binding sites (amino groups) of the branching agent either directly or by way of one or more spacers, each cleavable linker providing a distal binding site, the branching agent bound to the polymeric matrix; the at least one spacer positioned at anyone of the following locations: between the polymeric matrix and the branching agent, between binding sites of the distal branching agent and cleavable linkers.

One aspect of the invention relates to an insoluble support in particulate form comprising a polymeric matrix and constructs, the constructs covalently bound to the polymeric matrix, the constructs comprising three branching agents, four cleavable linkers and spacers; all branching agents selected from diaminoalkanoic acids comprising from 3 up to 10 carbon atoms comprising binding sites, two branching agents being second generation distal branching agents comprising binding sites, and one branching agent being the first generation proximal branching agent, the cleavable linkers covalently bound to the binding sites of the distal branching agents either directly or by way of one or more spacers, each cleavable linker providing a distal binding site; the first generation proximal branching agent bound to two second generation distal branching agents, the first generation proximal branching agent bound to the matrix, the spacers positioned at anyone of the following locations: between branching agents, between polymeric matrix and branching agents, between binding sites of the distal branching agents and linkers.

One aspect of the invention relates to an insoluble support in particulate form comprising a polymeric matrix and constructs, the constructs covalently bound to the polymeric matrix, the constructs comprising seven (7) branching agents, eight cleavable linkers and spacers; all branching agents selected from selected from diaminoalkanoic acids comprising from 3 up to 10 carbon atoms, four (4) branching agents being third generation distal branching agents comprising binding sites, two branching agents being second generation intermediate branching agents, one branching agent being the proximal branching agent, the cleavable linkers covalently bound to the binding sites of the distal branching agents either directly or by way of one or more spacers, each cleavable linker providing a distal binding site, each second generation intermediate branching agent bound to two third generation distal branching agents and to one first generation intermediate branching agent, the first generation intermediate branching agent bound to two second generation intermediate branching agents and the first generation proximal branching agent, the first generation proximal branching agent bound to the matrix; the spacers positioned at anyone of the following locations: between branching agents, between polymeric matrix and branching agents, between binding sites of the distal branching agents and linkers.

One aspect of the invention relates to an insoluble support in particulate form comprising a polymeric matrix and constructs, the constructs covalently bound to the polymeric matrix, the constructs comprising fifteen (15) branching agents, sixteen (16) cleavable linkers and spacers; all branching agents selected from diaminoalkanoic acids comprising from 3 up to 10 carbon atoms, eight (8) branching agents being fourth generation distal branching agents comprising binding sites, four (4) branching agents being third generation intermediate branching agents, two branching agents being second generation intermediate branching agents, one branching agent being the first generation proximal branching agent, the cleavable linkers covalently bound to the binding sites of the distal branching agents either directly or by way of one or more spacers, each cleavable linker providing a distal binding site, each third generation intermediate branching agent bound to two fourth generation distal branching agents and one second generation intermediate branching agent, each second generation intermediate branching agent bound to two third generation intermediate branching agents and to one first generation intermediate branching agent, the first generation intermediate branching agent bound to two second generation intermediate branching agents and the first generation proximal branching agent, the first generation proximal branching agent bound the matrix; the spacers positioned at anyone of the following locations: between branching agents, between polymeric matrix and branching agents, between binding sites of the distal branching agents and linkers.

One aspect of the invention relates to an insoluble support in particulate form comprising a polymeric matrix and constructs, the constructs covalently bound to the polymeric matrix, the constructs comprising thirty-one (31) branching agents, thirty-two (32) linkers and optional spacers; all branching agents selected from diaminoalkanoic acids comprising from 3 up to 10 carbon atoms, sixteen (16) branching agents being fifth generation distal branching agents comprising binding sites, eight (8) branching agents being fourth generation intermediate branching agents, four (4) branching agents being third generation intermediate branching agents, two (2) branching agents being second generation intermediate branching agents, and one branching agent being the first generation proximal branching agent, the linkers covalently bound to the binding sites of the distal branching agents either directly or by way of one or more spacers, each linker providing a distal binding site, each fourth generation intermediate branching agent bound to two fifth generation distal branching agents and one third generation branching agent, each third generation intermediate branching agent bound to two fourth generation intermediate branching agents and one second generation intermediate branching agent, each second generation intermediate branching agent bound to two third generation intermediate branching agents and one first generation proximal branching agent, and the proximal branching agent bound to the matrix; the spacers positioned at anyone of the following locations: between branching agents, between polymeric matrix and branching agents, between binding sites of the distal branching agents and linkers.

According to a further embodiment the construct does not contain arginine.

One aspect of the invention relates to an insoluble support in particulate form comprising secondary binding sites and a polymeric matrix, wherein said secondary binding sites are provided by cleavable linkers bound to constructs comprising at least one branching agent selected from aminoalkanoic acids comprising at least 2 amino groups and from 3 up to 10 carbon atoms, cleavable linkers and spacers, the branching agent positioned between the secondary binding sites and the polymeric matrix, the constructs coupled to the polymeric matrix covalently.

A further aspect relates to an insoluble support in particulate form comprising constructs, distal binding sites and a polymeric matrix, the constructs bound to the polymeric matrix, the constructs comprising cleavable linkers and at least two branching agents selected from aminoalkanoic acids comprising at least 2 amino groups and from 3 up to 10 carbon atoms, wherein (with the proviso that) the at least two branching agents comprise different numbers of binding sites, one branching agent being a distal branching agent providing the distal binding sites, the cleavable linkers covalently bound to the binding sites of the distal branching agent either directly or by way of one or more spacers and each cleavable linker providing a distal binding site, and at least one spacer being positioned between branching agents, between polymeric matrix and branching agents and between distal branching agents and cleavable linkers.

Yet a further aspect relates to an insoluble support (resin) in particulate form for use in solid phase peptide synthesis, solid phase morpholino oligomer synthesis and solid phase oligonucleotides synthesis, preferably solid phase peptide synthesis, comprising distal binding sites, the support comprising a homogeneous polymeric matrix and constructs, the constructs covalently bound to the polymeric matrix, wherein the constructs comprise at least one branching agent selected from diaminoalkanoic acids comprising from 3 up to 10 carbon atoms, cleavable linkers and at least one spacer coupled to at least one branching agent via an amide bond, the cleavable linkers providing the distal binding sites and the constructs selected from branched molecules of the formulas:

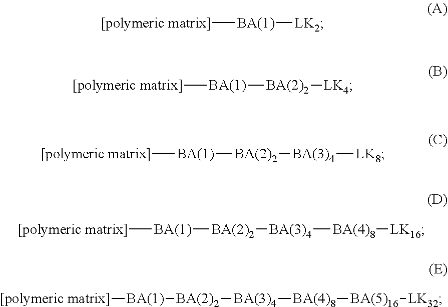

where BA is a branching agent and the integer denoting the generation of branching agents, and LK denoting a cleavable linker.

According to yet a further aspect, the constructs are selected from the molecules:

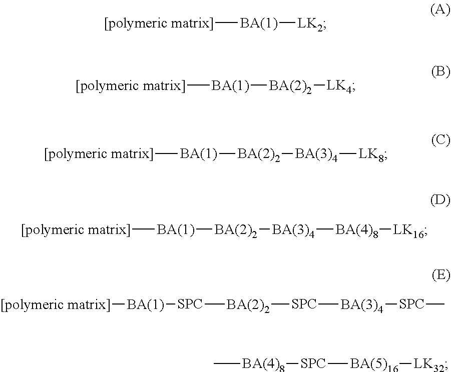

where BA is a branching agent and the integer denoting the generation of branching agents, LK denoting a cleavable linker, and SPC denoting any number of spacers (from at least one spacer) and where optionally any number of spacers (at least one spacer) may be positioned between the polymeric matrix and the first generation branching agent BA (1), and between cleavable linkers and last generation of branching agents (distal branching agents).

A still further aspect relates to an insoluble support (resin) in particulate form for use in solid phase morpholino oligomer synthesis, solid phase oligonucleotides synthesis and solid phase peptide synthesis, preferably solid phase peptide synthesis, comprising distal binding sites, the support comprising a homogeneous polymeric matrix and constructs, the constructs covalently bound to the polymeric matrix, wherein the constructs comprise at least one branching agent selected from diaminoalkanoic acids comprising from 3 up to 10 carbon atoms, cleavable linkers and at least one spacer coupled to at least one branching agent via an amide bond, the cleavable linkers providing the distal binding sites and the constructs selected from branched molecules:

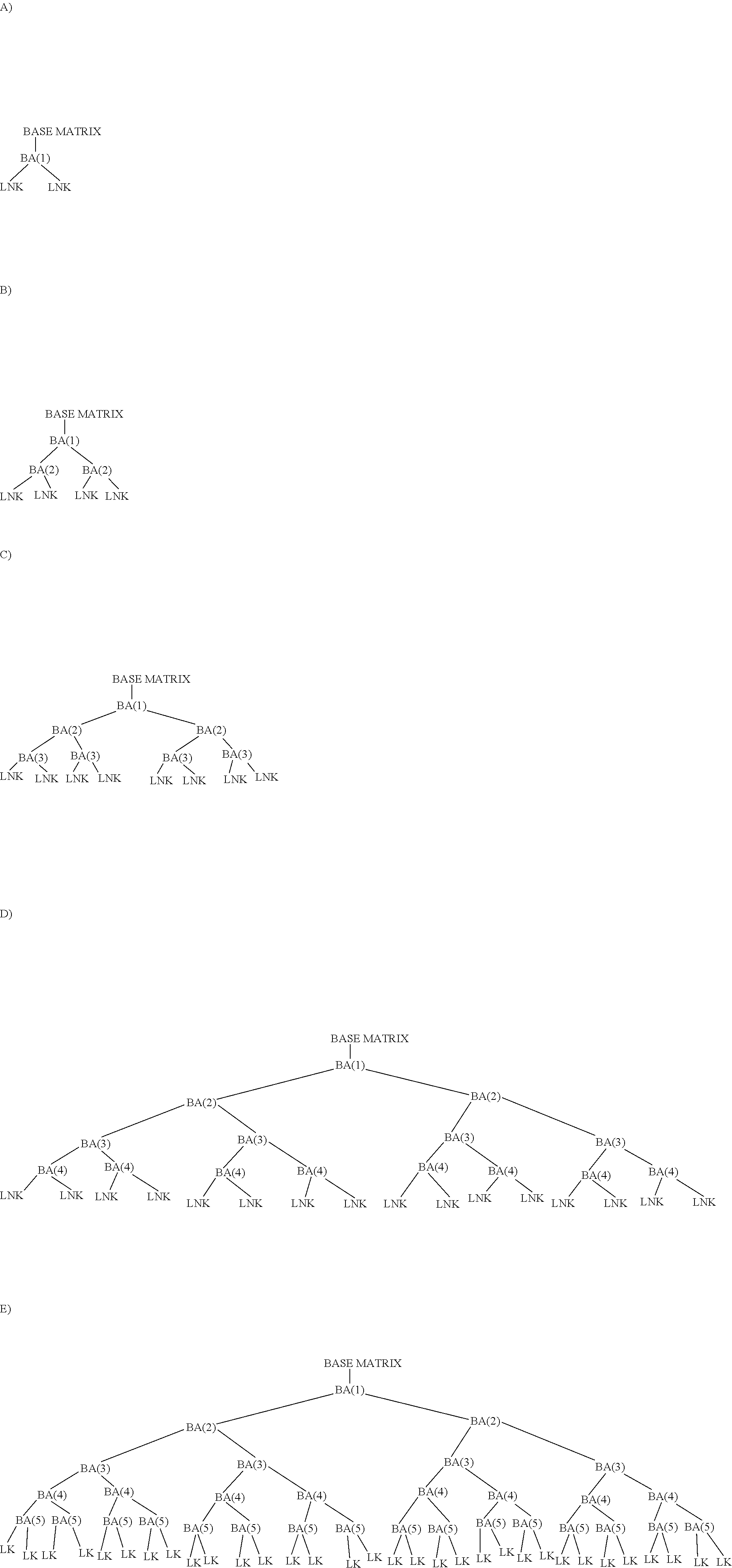

where BA is a branching agent and the integer denoting the generation of branching agents, LK denoting a cleavable linker, and where optional any number of spacers (at least one spacer) may be positioned between the polymeric matrix and the first generation branching agent BA (1), between branching agents, and between cleavable linkers and last generation of branching agents (distal branching agents).

Constructs of structure A contain only one first generation proximal branching agent. Constructs of structure B contain three branching agents, one first generation proximal branching agent and two second generation distal branching agents. Constructs of structure B have two layers or two generations of branching agents. Constructs of structure C contains seven branching agents, one first generation proximal branching agent, two second generation intermediate branching agents and four third generation distal branching agents, signifying three layers/generation of branching agents. Construct D contains fifteen branching agents, one first generation proximal branching agent, two second generation intermediate branching agents, four third generation intermediate branching agents and eight fourth generation distal branching agents, i.e construct D contains four layers (generations) of branching agents. Construct E contains thirty-one branching agents, one first generation proximal branching agent, two second generation intermediate branching agents, four third generation intermediate branching agents, eight fourth generation intermediate branching agents and 16 fifth generation distal branching agents, signifying the presence of five layers (generations) of branching agents.

According to an aspect the diaminoalkanoic acids (DAA) are selected from §-DAA(§) and §-DAA($), where DAA denotes a diaminoalkanoic acid, such as a diaminoalkanoic acids comprising from 3 up to 10 carbon atoms, suitably from 3 up to 8 carbon atoms; the character not in brackets, §, denoting alpha amine protecting groups and the characters in bracket, § and $, denoting side chain protection groups, where $ represents a protecting group cleaved under different cleavage conditions or not cleaved during the same cleavage step as protection group §.

The protection groups may be selected from Fmoc (fluorenylmethoxycarbony), Mtt (4-methyltrityl), Mmt (4-methyloxytrityl), Alloc (allyloxycarbonyl), Dde [N-(1-(4,4-dimethyl-2,6-dioxocyclohexylidene)ethyl)] or ivDde.

Protection group § may be Fmoc and $ may be selected from Mtt (4-methyltrityl), Mmt (4-methyloxytrityl), Alloc (allyloxycarbonyl), Dde [N-(1-(4,4-dimethyl-2,6-dioxocyclohexylidene)ethyl)] or ivDde.

According to an aspect all branching agents are selected from aminoalkanoic acids comprising at least 2 amino groups and from 3 up to 10 carbon atoms where both amines are protected by identical protecting groups.

According to an aspect, branching agents are exclusively selected from amine protected diaminoalkanoic acids, preferably diaminoalkanoic acids comprising from 3 up to 10 carbon atoms, suitably from 3 up to 8 carbon atoms, wherein said amine protected diaminoalkanoic acids are selected from diaminoalkanoic acids comprising amine protecting groups which are cleaved during the same cleavage step and/or cleaved under same/similar cleavage conditions, and diaminoalkanoic acids comprising amine protecting groups leaved under different cleavage conditions or not cleaved during the same cleavage step.

The polymeric matrix may also be denoted as a resin. The polymeric matrix may comprise a polymeric network such as a cross-linked polymeric network. The insoluble support but also the polymeric matrix is preferably in particulate form. Typically, the insoluble support is in particulate form, such as in the form of beads, of a size from about 1 μm up to about 1000 μm, suitably from about 5 μm up to about 750 μm, from about 10 μm up to about 300 μm or in the range of from about 50 μm up to about 200 μm.

According to a further aspect, the polymeric matrix is selected from homogeneous polymeric matrices. Homogeneous polymeric matrices herein denote polymeric matrices where binding sites are distributed throughout the matrix, preferably binding sites are distributed homogeneously (essentially evenly) throughout the polymeric matrix. Homogeneous denotes that important parameters of the matrix, such as cross-linking and distribution of (primary) binding sites, are homogeneously distributed throughout the matrix.

There is a certain type of inhomogeneous resins for solid phase peptide synthesis where the binding sites are not homogeneously distributed within the resin. Rather, the binding sites are concentrated at or close to the surface of the resin bead forming a shell and where the core of the bead essentially is devoid of binding sites.

Homogeneous polymeric matrices do not embrace inhomogeneous resins having essentially all binding sites at or close to the surface. Thus, homogeneous polymeric matrices in particulate form (essentially spherical/bead form) do not comprise a shell at or close to the surface (phase boundary) where essentially all binding sites are located and a core essentially devoid of binding sites.

According to a further aspect, the polymeric matrix is selected from polymeric matrices obtained by emulsion polymerization comprising at least styrene and divinylbenzene (DVB). Preferably, the DVB is present during emulsion polymerization in an amount of below 4.0 wt %, preferably below 3.5 wt %, preferably below 3.0 wt %. Preferably DVB is present in an amount from about 0.5 wt % up to about 2.5 wt %.

The insoluble support may also be characterized by structural features and features relating to the formation of the insoluble support. Thus, an aspect related to a insoluble support in particulate form comprising constructs as defined by any one of the aspects presented herein, the insoluble support obtained by the provision of a polymeric matrix comprising primary binding sites, wherein the constructs are covalently bound to the primary binding sites, the constructs providing secondary binding sites such that primary binding sites provides at least two secondary binding sites, where the construct comprises at least one branching agent, linkers and optional spacers, and where the construct is formed by the implementation of a solid phase synthesis protocol, the protocol comprising recurrent synthesis steps comprising the coupling of at least one branching agent to the polymeric matrix and optionally to other branching agent.

A still further embodiment relates to an insoluble support (resin) in particulate form for use in solid phase peptide synthesis comprising distal binding sites, the support comprising a polymeric matrix and constructs, the constructs covalently bound to the polymeric matrix, wherein the constructs comprise at least one branching agent selected from aminoalkanoic acids comprising at least 2 amino groups and from 3 up to 10 carbon atoms, cleavable linkers and at least one spacer coupled to at least one branching agent via an amide bond, the cleavable linkers providing the distal binding sites and being covalently bound to the at least one branching agent; the construct formed by a synthesis comprising providing a polymeric matrix as the solid phase comprising primary binding sites, branching agents, linkers and spacers, the construct formed by at least one coupling step, preferably recurrent coupling steps, said step or steps comprising at least the coupling of at least one branching agent, at least one spacer, and cleavable linkers to the branching agent.

A further embodiment relates to a method for forming an insoluble support in particulate form for use in solid phase peptide synthesis comprising secondary (distal) binding sites, the insoluble support comprising a polymeric matrix and constructs, the constructs comprising cleavable linkers and at least one spacer, the method comprising providing a polymeric matrix in particulate form comprising primary binding sites, wherein the construct is formed by a solid phase synthesis protocol, the solid phase protocol comprising at least one reaction steps where a branching agent selected from amino protected aminoalkanoic acids comprising at least 2 amino groups and from 3 up to 10 carbon atoms is coupled by an amide bond to either of primary binding sites of the polymeric matrix, a spacer or a branching agent, the protocol further comprising a reaction step where a cleavable linker is coupled to branching agents.

The insoluble support is preferably synthesized by the provision of a polymeric matrix and the coupling of the relevant compounds (branching agent, spacers and cleavable linker) by way of the formation of amide bonds and using a solid phase synthesis protocol. Preferably all compounds comprise the necessary functional groups for the provision of amide bonds when coupled to each other. Typically, all compounds comprise at least one amine/amino group and a carboxylic acid group/carboxyl group. As presented throughout this specification, the branching agent is selected from aminoalkanoic acids comprising at least 2 amino groups and from 3 up to 10 carbon atoms. Preferably, the spacers are selected from compounds comprising two binding sites, one being an amine/amino group and the other being a carboxylic acid group. Preferably, the spacers are selected from amino acids and derivatives thereof having two binding sites, i.e. one amine group and one carboxylic acid group.

It should be noted that all reactive groups of the relevant compounds need suitable protective groups save the carboxylic acid group. More specifically any amine/amino groups need to be protected by suitable protection groups.

A reaction cycle in solid phase peptide synthesis protocol includes at least the following steps:
a) provision of a compound comprising at least one amine group and a carboxylic acid group where relevant amine groups are protected by relevant protecting groups. Suitable protecting groups are covalently bound to the amine. If the compound also comprises a side chain having a reactive group such side chain reactive group is also protected. The compound comprising at least a protected amine group and a carboxylic acid group is coupled to the primary binding sites of the polymeric matrix in the presence of suitable coupling agents thereby forming an amide bond. If the compound is an amino acid the α-amine is always protected and if needed any reactive group of the side chain such as the ε-amine of lysine.
b) after coupling of the relevant compound to the polymeric matrix, the excess reagents and side products are separated
c) next a further compound comprising at least one amine group and a carboxylic acid group where relevant amine groups are protected by relevant protecting groups is coupled to the free amine group of the previous compound coupled to the polymeric resin in step.

When the last generation of branching agents have been coupled, cleavable linkers are coupled to the distal branching agents.

The process of synthesizing a polypeptide may start with the provision of a polymeric matrix, modifying the polymeric matrix in accordance to the present invention, and once the insoluble support of the invention has been formed continue with the synthesis of any relevant target polypeptide.

for synthesizing the insoluble support may comprise the following steps:
a) optionally coupling at least one spacer molecule to the primary binding sites,
b) coupling a primary branching agent which preferably is selected from diaminoalkanoic acids, the two amine groups protected by protecting groups, preferably diaminoalkanoic acids comprising from 3 up to 10 carbon atoms, suitably from 3 up to 6 carbon atoms, such as diaminopropionic acid, diaminobutyric acid, diaminopentanoic acid and diaminohexanoic acid to the primary binding sites of the polymeric matrix, or optionally coupling the branching agent to a spacer molecule,
c) removal of the protecting groups,
where steps a), b) and c) may be repeated, and
d) coupling of linkers to unprotected amine groups of the distal branching agents.

The two amines of the above diaminoalkanoic acids of step b) are suitably protected by protecting groups selected from amine protecting groups which are cleaved during the same cleavage step and/or cleaved under same/similar cleavage conditions, and diaminoalkanoic acids comprising amine protecting groups leaved under different cleavage conditions or not cleaved during the same cleavage step.

According to an embodiment, all diaminoalkanoic acids have protecting groups cleaved off during same deprotection step. Thus, suitably the alpha amine and the side chain amine are protected by the same protecting group.

A further embodiment of the invention relates to a construct. The construct and the polymeric matrix support form the insoluble support of the invention.

According to an aspect, the construct and the insoluble support can be formed by using a solid phase protocol. The solid phase may be a base-insoluble support. The construct is the entity increasing the number of binding sites, i.e. being the entity coupled to the primary binding sites of the insoluble support ultimately providing a plurality of secondary binding sites. The construct comprises at least one branching agent as disclosed herein. As presented above, the construct may comprise two or more layers/generations of branching agents. Distal or intermediate branching agents of the construct are coupled to the proximal branching agent optionally through spacers. A preferred procedure to form the construct and the modified insoluble support is the application of a solid phase protocol. According to an aspect the branching agent is selected from diaminoalkanoic acids, and the construct can be synthesized using established reaction protocols in solid phase peptide synthesis (SPPS). Once a target construct is generated the construct can be cleaved off from the insoluble support. Alternatively, the construct may be synthesized by a liquid phase reaction protocol.

The construct may be regarded as a branched polymer, or a branched polymer comprising amide couplings. The construct may also be regarded as a branched peptide, or peptide-like polymer comprising spacers.

According to one aspect if ϕ denotes the number of binding sites of a branching agent, then the number of secondary binding sites of a construct as a function of the layers may be denoted as: λ=(ϕ−1)ⁿ, where ϕ denotes the number of binding sites of the branching agent, and n denotes the number of layers of branching agents in the construct, provided that all branching agents of the construct have identical number of binding sites. If the construct only contains one branching agent and said branching agent is lysine (lysine having three binding sites the number of secondary binding sites λ provide by this construct is λ=(3−1)¹: two secondary binding sites.

Hence, modifying primary binding sites with lysine as branching agent (and construct) providing only one layer or generation of branching agents would render two secondary binding sites for every primary binding site of the polymeric matrix.

The following aspect of the insoluble support relates to insoluble supports comprising one, two, three, four and five layers (generations) of branching agents, the binding agents referred to as primary, secondary, tertiary, quaternary and quinary branching agents. Primary branching agents form the first lever (generation), secondary branching agents form the second layer, tertiary branching agents form the third lever and so on. Should the branching agent contain three binding sites, e.g. lysine, then the number of secondary binding sites provided equals 2ⁿ, where n denotes number of levels. Thus, a insoluble support comprising lysine as branching agent and further comprising five levels of quinary lysine branching agents will provide 2ⁿ, i.e. 2⁵ or 32 secondary binding sites from every primary binding site.

Within the ambit of the invention are also insoluble supports comprising more than five levels of branching agents. However, for every level the number of secondary binding sites increases exponentially. The availability of secondary binding sites for peptide synthesis may be enhanced by the introduction of spacers (spacer molecules) specifically between branching agents.

A further aspect relates to an insoluble support/polymeric matrix comprising primary binding sites where proximal branching agents are covalently bound to the primary binding sites optionally by spacer molecules, and wherein the primary branching agent provides at least two secondary binding cites.

The construct may not only comprise one type of branching agent but can comprise different types of branching agents. The construct may comprise branching agents with different numbers of binding sites and/or branching agents with the same number of binding sites but different branching agents.

A further aspect relates to any insoluble support disclosed herein used as the solid phase in any solid phase synthesis protocol. The insoluble support is preferably implemented as the solid phase in solid phase peptide synthesis.

A further aspect relates to the use of the insoluble support as the solid phase for synthesizing peptides and polypeptides in a solid phase peptide synthesis.

### Branching Agents

As presented above the construct is a molecule enabling the provision of secondary/distal binding sites. In its simplest incarnation, the construct comprises one branching agent. The most generic definition of the branching agent is an organic molecule comprising at least three binding/coupling sites. A binding/coupling site is a functional group which is able to form a covalent bond with a binding/coupling site of another molecule (e.g. two binding sites of two different molecules form a coupling in form of a covalent bond, spacer-spacer, spacer-branching agent, coupling between two branching agents, e.g. secondary (intermediary/distal) branching agent-primary (proximal) branching agent, primary (proximal) branching agent and primary binding site of a polymeric matrix).

Typically, the branching agent comprises functional groups selected from amines, carboxylic acids, alcohols, ketones and aldehydes. Alternatively, the functionals groups are selected such that the coupling generates amides, ethers and esters. Preferably, the branching agents comprise functional groups selected from amines and carboxylic acids. Preferred binding sites of branching agents are amines and carboxylic acids. The amines are preferably primary amines.

The branching agent may be selected from any natural amino acid or derivative of an amino acid providing at least three coupling sites.

According to a preferred aspect, the branching agent is selected from aminoalkanoic acids comprising at least 2 amino groups and from 3 up to 10 carbon atoms.

According to a further aspect, the branching agent is selected from aminoalkanoic acids comprising at least 2 but not more than 3 amino groups and from 3 up to 10 carbon atoms.

According to a still further aspect, the branching agent is selected from diaminoalkanoic acids comprising from 3 up to 10 carbon atoms, and preferably selected from diaminoalkanoic acids comprising from 3 up to 8 carbon atoms, such as from 3 up to 6 carbon atoms.

According to a further aspect, the branching agent is selected from 2,3-diaminopropionic acid (Dpr), 2,4-diaminobutyric acid and 2,5-diaminopentanoic acid (ornithine), 2,6-diaminohexanoic acid, suitably the branching agent is selected from 2,4-diaminobutyric acid and 2,5-diaminopentanoic acid (ornithine), 2,6-diaminohexanoic acid.

According to a further aspect, the branching agent is selected from (lysine analogs) such as diaminopropionic acid, diaminobutyric acid and diaminopentanoic acid, diaminohexanoic acid.

According to an aspect, the branching agent is lysine (diaminohexanoic acid) and more specifically 2,6-diaminohexanoic acid.

Branching agent may comprise one or more chiral carbon centers (asymmetric carbons). Such chiral carbons may either be in the S or R configuration. A branching agent comprising chiral carbons may have all chiral carbons in either the S or R configuration or if multiple chiral carbons are present exhibiting both S and R configuration.

Amino acid-based branching agents but also amino acid-based spacers may be in the D or L form. Most amino acids are chiral (contain a chiral carbon: notably the alpha carbon). Glycine does not have a chiralasymmetric carbon atom. The constructs may comprise branching agents and optional spacers which are in the D and/or L form. Hence, the construct may comprise both amino acids in D and L form. According to an embodiment, all chiral carbons of relevant amino acid-based branching agents and optional spacers are in their L-form.

### Spacers

Spacers may be present between branching agents, between a branching agent and the primary binding site of the polymeric matrix and between the cleavable linkers and the branching agents. A spacer needs to contain two binding/coupling sites/functional groups available for forming covalent couplings.

The binding/coupling/functional group of a spacer may be selected from amines, carboxylic acids, alcohols, ketones and aldehydes. Preferably, the functional groups are selected from amines, carboxylic acids and alcohols. Preferably, the functional groups of the spacers form amides, and ethers. Preferably, the spacer can be selected from any molecule having two functional groups which both can form amide bonds. Suitable spacers are any molecule having two functional groups which can form amide bonds and be applied in a solid phase synthesis protocol.

According to a preferred aspect spacers are selected from amino acids or amino acid derivatives. More specifically, spacers are selected from amino acids having one amine.

Useful spacers may be amino hydrocarbon carboxylic acids, i.e. any type of hydrocarbon chain comprising one amine and one carboxylic acid. Preferably, the amine and carboxylic acids are at the ends of the hydrocarbon chain.

The spacers may be selected from amino acids comprising one carboxylic acid group and one amine group. Suitable amino acids may also comprise aliphatic hydrocarbons. Exemplified amino acid spacers are glycine, alanine, beta-alanine, isoleucine, leucine and any derivatives thereof.

According to an aspect the spacer is selected from aminoalkanoic acids, specifically monoaminoalkanoic acids comprising from 1 up to 10 carbon atoms, such as glycine, alanine, beta alanine, valine, isoleucine, leucine, beta leucine, ε-aminopropionic acid, 4-aminobutanoic acid (4-aminobutyric acid), 5-aminopentanoic (5-aminovaleric acid) acid, ε-aminohexanoic acid (ε-aminocaproic acid), 7-aminoheptanoic acid (7-aminoenanthic acid), 8-aminooctanoic acid (8-aminocaprylic acid), 9-aminononanoic acid, 10-aminodecanoic acid.

A preferred spacer is glycine.

Any number of spacers may be applied to the construct. Spacers may be positioned between branching agents, between a branching agent and a primary binding site of the insoluble support and between the construct linker and the branching agent.

The hydrophilicity/hydrophobicity of the construct and insoluble support can be modulated by the choice of branching agent and/or spacer.

According to an aspect, PEG spacers are positioned between the last layer of branching agents (also referred to as distal branching agents) and the linkers and/or between branching agents.

PEG spacers may preferably be provided as 2-[2-[2-(Fmoc amino) ethoxy]ethoxy] acetic acid (AEEA). AEEA spacers have the advantage that they are easily integrated in a solid phase peptide synthesis protocol.

### Cleavable Linker

The cleavable linkers are positioned between the secondary binding sites and the most distal branching agents. The secondary binding site is typically integrated with the linker. Thus, the linker may also be referred to as the secondary binding site. By distal is meant furthers away from the polymeric matrix. The most distal layer of branching agents signifies the layer farthest away from the base of the polymeric matrix. The linker chosen is dependent on the chemistry used for synthesizing the peptide and especially the cleavage conditions targeted. Also, the type of polymeric matrix may have an implication on the selection of linker. Linkers may be selected from PAM, Wang, Rink such as Rink amide and Rink acid, PAL, Ramage, Sieber, MBHA, Trityl chloride, Oxime, HMPA, HMPB, DHP, Weinreb aminomethyl.

According to an aspect, the cleavable linker is not a photolytically cleavable linker.

### Polymeric Matrix

The insoluble support comprises a polymeric matrix and constructs covalently attached to the polymeric matrix, typically comprising binding sites, also referred to as primary binding sited. The constructs are covalently bound to the biding sites of the polymeric matrix. The polymeric matrix may be modified to comprise any type of binding sites suitable for the coupling of the constructs. It should be noted that the primary binding sites of a polymer matrix are consumed during the formation of the insoluble support of the invention. Thus, the primary binding sites of the polymeric matrix are used to covalently bind the constructs. Ideally, all primary binding sites of the polymeric matrix are consumed by the covalent coupling of constructs signifying that the insoluble support of the invention does not have a polymeric matrix with primary binding sites. The polymeric matrix is typically a polymer network usually comprising polymers cross-linked to a degree providing structural integrity of the polymeric matrix particles while enabling a significant solvation of the polymer network. The polymeric matrix is preferably selected from polymeric matrices comprising binding sites distributed throughout the polymeric matrix. The polymeric matrix can be referred to as a homogeneous polymeric matrix. The polymeric matrix may also be referred to as a polymeric network or resin.

According to an aspect, the polymeric matrix is selected from homogeneous polymeric matrices. Homogeneous polymeric matrices herein denote polymeric matrices where binding sites are distributed throughout the matrix, preferably binding sites are distributed homogeneously (essentially evenly) throughout the polymeric matrix. Homogeneous in relation to polymeric matrices denotes that certain parameters of the matrix, such as cross-linking and distribution of (primary) binding sites, are homogeneously distributed throughout the matrix.

There is a certain type of inhomogeneous resins for solid phase peptide synthesis where the binding sites are not homogeneously distributed within the resin. Rather, the binding sites are concentrated (confined) at or close to the surface of the resin bead forming a shell and where the core of the bead essentially is devoid of binding sites.

Homogeneous polymeric matrices do not embrace inhomogeneous resins having essentially all binding sites at or close to the surface.

According to a further aspect, the polymeric matrix is selected from polymeric matrices, specifically homogeneous matrices, obtained by emulsion polymerization comprising at least styrene and a cross-linker preferably divinylbenzene (DVB). Preferably, the cross-linker is present during emulsion polymerization in an amount of below 4.0 wt %, preferably below 3.5 wt %, preferably below 3.0 wt %. Preferably DVB is present in an amount from about 0.5 wt % up to about 2.5 wt %.

According to an aspect the polymeric matrix is selected from homogeneous cross-linked polystyrene-based matrices, preferably homogeneous cross-linked polystyrene-based matrices comprising below 4.0 wt % of cross-linking agent, preferably below 3.5 wt % of cross-linking agent, preferably below 2.0 wt % of a cross-linking agent.

Polystyrene-based matrices in addition to cross-linking preferably with DVB, may be modified by various monomers such as hydroxyethylstyrene and polyethylene glycol.

The polymeric matrix may be formed by emulsion polymerization. Particularly useful polymeric matrices are styrene-based matrices cross-linked with divinyl benzene (DVB). Styrene-based matrices cross-linked with DVB preferably have a content of DVB below 4.0 wt %, preferably below 3.5 wt %, preferably below about 3.0 wt %

The polymeric matrix may also be denoted as a resin. The polymeric matrix may be selected from polystyrene, polyacrylate, polyacrylamide, polyamide or polyethylene glycol. The polymeric matrix may also be a hybrid of at least two different types of polymers. Thus, the polymeric matrix may be polystyrene which is modified by another type of polymer such as ethylene glycol, polyacrylate, polyamide or polyacrylamide.

The insoluble support is preferably in particulate form serving as the solid phase. The particulate form of the insoluble support is suitably of spherical shape. Thus, the insoluble support is suitably of spherical shape and may be denoted as bead form or simply bead. A bead is preferably spherical. The size of the insoluble support (spherical beads) may range from about 1 μm up to about 2000 μm, such as up to about 1000 μm, suitably from about 5 μm up to about 750 μm, such as from about 20 μm up to about 500 μm, from about 50 μm up to about 300 μm, specifically from about 50 to 150 μm. Not only bead size matters, but also bead size distribution should be taken into consideration.

The polymeric matrix is usually substituted. Substitution may be accomplished by direct incorporation of the substrate onto the polymeric matrix by way of e.g. an electrophilic aromatic substitution reaction or copolymerization of the substituted polymer with the polymer of the polymer matrix, e.g. styrene.

Examples of polymeric matrices are polystyrene based matrices modified with aminomethyl (AM or AMS), 4-methylbenzhydryl amine (MBHA), cross-linked polystyrene-based matrices modified with PEG suitably by grafting PEG to the polystyrene base matrix such as Tentagel™ resins, HypoGel® and Tentage®, polyethylene glycol (PEG) based resins/matrices such as ChemMatrix® and NovaPEG, aminomethylated polystyrene-based matrices partially derivatized methyl-PEG-p-nitro-phenyl-carbonate e.g. NovaGel™, polystyrene-based matrices co-polymerized with hydroxyethylpolystyrene and polyethylene glycol (PEG) e.g NovaSyn™

### Construct

The construct may be referred to as a branched molecule and in a certain sense also as a branched polymer as the construct preferably comprises repetitive units, such as branching agents and optional spacers. As presented the construct comprises at least a branching agent and optional spacers. Theoretically, there is no upper limit of number of branching agents comprised in the construct. For most applications the upper number of branching agents in the construct is about 150. Most constructs will comprise a number of branching agents from 1 up to about 150, from 1 up to about 100, such as from about 1 up to about 80. The branching is induced by the branching agent. According to an aspect, the branching agent and spacers are selected from amino acids or derivatives of amino acids. A construct comprising branching agents and spacers selected from amino acids may be denotes as a branched peptide. Hence, certain constructs may be referred to as branched peptides or polypeptides. The molecular weight, excluding linkers, of the construct may start from about 100 g/mol up to about 200000 g/mol, up to about 100000 g/mol, up to about 10000 g/mol. The exemplified 2×(Gly) construct has a molecular weight of around 1245 g/mol, the 16×(Gly) construct (both exemplified herein) a mole weight of around 3375 g/mol. The construct comprises cleavable linkers coupled to the distal branching agent(s) suitable for the targeted polypeptide and peptide chemistry, preferably taking due account to the capping strategy.

### General Disclosure of the Method for Forming the Insoluble Support and the Construct of the Invention

The insoluble support may be formed by the provision of a commercially available polymeric matrix and the gradually synthesis of the construct e.g. by a solid phase peptide synthesis protocol: a divergent synthesis protocol. Alternatively, the insoluble support may be formed by the provision of a commercially available polymeric matrix and the coupling of the complete construct, a convergent synthesis protocol. Preferably, the insoluble support is formed by a solid phase synthesis protocol comprising the consecutive addition of the relevant chemical compounds, specifically branching agents, linkers and spacers.

The construct is preferably provided by a process protocol similar to a protocol for the provision of the insoluble support albeit applying a linker between the construct and the resin enabling the cleavage of the construct from the resin. A construct cleaved off from the resin may be used for modifying a polymeric matrix. According to an aspect the branching agents and spacers are selected form amino acids and derivatives of amino acids, providing a construct which can be referred to as a peptide, typically branched peptide, or branched polypeptide.

Any solid phase peptide synthesis chemistry may be applied for the synthesis of the construct including a variety of amine protection and side chain protection chemistries and peptide bond formation activation chemistries.

Reagents for solid-phase synthesis are readily available from commercial sources. Solid-phase synthesis procedure usually comprises the following re-current operations: deprotection of N-terminal alpha amine protecting group and/or deprotection of side chain amine protecting group of amino acids covalently coupled to the solid phase, coupling of N-terminal amine protected and side chain amine protected amino acids, optional capping of un-reacted amino acids, e.g. acetylation, and washing steps for displacement of by-products. Additionally, the following operations may be applied: blocking interfering groups and protecting amino acids during reaction.

According to an aspect the branching agents are selected from aminoalkanoic acids comprising at least 2 amino groups and from 3 up to 10 carbon atoms, preferably diaminoalkanoic acids comprising from 3 up to 10 carbon atoms, such as diaminoalkanoic acids selected from lysine analogs including diaminopropionic acid, diaminobutyric acid, diaminopentanoic acid, diaminohexanoic acid and diaminoheptanoic acid.

The amine groups of the diaminoalkanoic acids may be protected by a variety of protective groups including fluoren-9-ylmethyloxycarbonyl (Fmoc), Tert-butoxycarbonyl (Boc), tert-butyl tBu and trityl-based protection groups including 4-methyltrityl (Mtt) and 4-methoxytrityl (Mmt).

The Fmoc strategy has an advantage over the Boc strategy that Fmoc can be removed under milder reaction conditions. The Fmoc strategy enables the usage of base sensitive N-terminal amine group protection groups and acid sensitive side-chain protection groups. The Fmoc N-terminal protection group is removed under basic conditions not removing acid-labile side-chain protection groups.

According to an aspect the amine groups of the diaminoalkanoic acids (DAA) are protected by groups cleaved off under similar cleavage conditions, typically inferring that the amine groups of the diaminoalkanoic acids are protected by the same (identical) protecting group. However, the respective amine groups of the diaminoalkanoic acids, the N-terminal, alpha amine group and the amine group of the side chain, may also be protected by protecting groups cleaved under different cleavage conditions, such as different pH.

A diaminoalkanoic acid where the amines are protected by same protection group is also referred to as §-DAA(§), where § denotes the alpha amine protection group and (§) denotes the epsilon amine protection group.

A diaminoalkanoic acid where the amines are protected by different protection group is also referred to as $-DAA(€), where $ denotes the alpha amine protection group and (€) denotes the side chain or orthogonal amine protection group. DAA can be lysine (K).

The diaminoalkanoic acid protection groups may be selected from Mtt (4-methyltrityl), or Mmt (4-methyloxytrityl), Alloc (allyloxycarbonyl), Dde [N-(1-(4,4-dimethyl-2,6-dioxocyclohexylidene)ethyl)] or ivDde.

Protection group § may be Fmoc and $ may be selected from Mtt (4-methyltrityl), or Mmt (4-methyloxytrityl), Alloc (allyloxycarbonyl), Dde [N-(1-(4,4-dimethyl-2,6-dioxocyclohexylidene)ethyl)] or ivDde.

According to an aspect all aminoalkanoic acids of a construct are protected by protection groups which protection groups are essentially all simultaneously cleaved off during the same cleavage step, e.g. the two amine groups (of all diaminoalkanoic acids) are all protected by the same protective group such as Fmoc, e.g. §-DAA(§). If only aminoalkanoic acids having all amines protected by same protection group are implemented for synthesizing the construct, then e.g. both the alpha and epsilon amine (in case the aminoalkanoic acids are diaminoalkanoic acids) will be coupled to the same (identical) compound, e.g. branching agent, spacer or linker.

According to an aspect at least one aminoalkanoic acid is used as a branching agent (during synthesis of the construct of an insoluble support) having the respective amine group protected by protection groups cleaved off under different cleavage conditions (such as different pH), e.g. $-DAA(€). By implementing aminoalkanoic acids having amine protecting groups cleaved under different conditions (typically under separate cleavage steps), it is possible to couple different compounds to the alpha and side chain amine.

Constructs can be formed using any combinations of different diaminoalkanoic acids and any ratios of diaminoalkanoic acids having amine group protecting groups cleaved under same cleavage step (and/or cleaved under essentially same cleavage conditions), e.g. §-DAA(§), and diaminoalkanoic acids having amine protecting groups cleaved off under different cleavage conditions, e.g. $-DAA(€). A construct can be formed by exclusively using diaminoalkanoic acids where both amine groups (N-terminal, alpha amine groups and side chain amine group, such as the epsilon amine group) are protected by identical protection groups. For example, both amine groups of all diaminoalkanoic acids are Fmoc protected. Alternatively, a construct can be formed by exclusively using diaminoalkanoic acids where the respective amine groups are protected by different protection groups cleaved off under different cleavage conditions, typically different pH. For example, one amine group of a diaminoalkanoic acid is protected by Fmoc while the other amine group is protected by e.g. a Mtt or Mmt-group. Insoluble support 7× linear disclosed in the experimental section is synthesized by first forming a main chain exclusively using lysine as branching agent where the amine groups of each lysine are protected by one Fmoc group and one Mtt-group and thereafter coupling Fmoc-Lys(Fmoc) to the side chains of the lysines of the main chain For the exemplified 7× insoluble support the main chain contains 3 lysines (provided as e.g. Fmoc-Lys(Mtt)). Any odd number of binding sites of a construct (e.g. 3, 5, 7, 9, 11, etc) can be provided by coupling any number of e.g. Fmoc-Lys(Mtt) to the polymeric matrix (1, 2, 3, 4, 5, 6, etc.), thereby forming main chains containing from 1 up to any number of Fmoc-Lys(Mtt). The main chain may be denoted Fmoc-Lys(Mtt)-[Lys(Mtt)]_{X}-Polymeric matrix, where X is 0 and up to an integer. X may be from 0 up to 50. In this context main chain is a chain where consecutive lysines are coupled to each other by way of the alpha amine.

In general, a linear construct can be formed by providing a polymeric matrix and consecutively coupling branching agents exclusively selected from aminoalkanoic acids having the N-terminal amine and the side chain amine protected by protecting groups cleaved under different cleavage conditions (e.g. different pH) and typically cleaved during different cleavage steps, and optionally spacers, thereby forming main chains. After the formation of the main chains, the side chain protecting groups are cleaved under suitable cleavage conditions. In a subsequent step, branching agents exclusively selected from aminoalkanoic acids having the N-terminal amine and the side chain amine protected by protecting groups cleaved under same cleavage conditions effectively being identical protection groups. In a further reaction step all remaining amine protecting groups are cleaved and subsequently suitable linkers are coupled to all de-protected amine groups.

A further aspect relates to a synthesis scheme where branching agent of the format $-DAA(€) are used for providing the main chain optionally with spacers between the branching agents and between polymeric matrix and first generation branching agent. In a further step the (€) protection groups are removed by suitable cleavage conditions. Subsequently, branching agents of the format §-DAA(§) are coupled. If no more steps are implemented where branching agents are coupled so calledlinear constructs are formed. 7× linear represents of a linear construct.

For certain constructs, specifically non-symmetrical constructs also referred to herein as linear constructs, the side chain amine of the branching agent, e.g. lysine, is protected by Mtt, Mmt, alloc, Dde or ivDde.

According to an aspect the construct is synthesized on an appropriate polymeric matrix as solid phase by the application of a solid phase synthesis protocol comprising the use of a branching agent selected from diaminoalkanoic acids comprising from 3 up to 10 carbon atoms where both the N-terminal amine group (alfa amine group) and the amine group of the sidechain of said branching agents are protected by groups cleaved off at similar reaction conditions, i.e the protective groups are cleaved off simultaneously.

Where applicable, amino acid type spacers such as spacers selected from amino acids comprising one carboxylic acid group, one amine group and aliphatic hydrocarbons are Fmoc protected.

According to a further aspect the construct is synthesized on an appropriate polymeric matrix as solid phase by the application of a solid phase synthesis protocol comprising the use of a branching agent selected from diaminoalkanoic acids comprising from 3 up to 10 carbon atoms where the N-terminal (alpha amine group) and the amine group of the sidechain, respectively, are protected by two different protective groups cleaved under different cleavage conditions such as different pH. Preferably, the protective groups are selected from acid-labile and base-labile protective groups, suitably Fmoc, Mtt and Mmt. By the coupling of a base-sensitive Fmoc protection group and an acid-sensitive Mtt or Mmt group to the respective amine groups of a branching agent, e.g. diaminoalkanoic acid, different chemical moieties can be coupled to the same branching agent.

Symmetrical constructs are formed when both the alpha and side chain amine groups have protective groups which are cleaved essentially simultaneously during the same cleavage step. Non-symmetrical, linear constructs are formed when the respective amine groups of at least one branching agent, e.g. diaminoalkanoic acid, are protected by two different protective groups cleaved under different cleavage conditions such as different pH, e.g. Fmoc and Mtt or Mmt.

Preferably, both the alpha and the side chain amine groups are protected with same protective groups, such as Boc or Fmoc, preferably Fmoc. Preferably, both the alpha and epsilon amine groups of the lysine are Fmoc protected.

The 7× linear is a non-symmetrical, linear construct. A non-symmetrical construct is formed if the reaction conditions are such that the alpha and epsilon amine groups of at least one lysine are coupled to different chemical moieties, e.g. a spacer and a branching agent (lysine). For achieving that the alpha and epsilon amine groups of the lysine are coupled to different chemical moieties, the alpha and epsilon amine groups of the lysine must have protective groups which are cleaved at different reaction condition. One way of achieving this is to have one acid-labile protective group and a base-labile protective groups. Thus, one amine group of lysine may be protected by Fmoc which is base-labile while the other amine group is protected by an acid-labile protection group like Mtt and/or Mmt.

A lysine branching agent comprising Fmoc protection of the N-terminal and Mtt or Mmt protection of the side chain amine may be coupled to a further branching agent and a spacer or a linker respectively. The synthesis of a construct may incorporate diaminoalkanoic acids comprising two Fmoc groups (i.e. both N-terminal and sidechain amine are Fmoc protected) and/or diaminoalkanoic acids comprising one Fmoc group and an Mtt and/or Mmt-group.

Fmoc groups are typically removed by the application of a base, such as a heterocyclic amine, preferably piperidine. Mtt groups may be cleaved under acid conditions such as by application of a solution comprising dichloromethane (DCM) and trifluoroacetic acid (TFA). Boc protecting groups are base-labile (sensitive) and are preferably removed by the application of TFA.

Amino acid coupling chemistries include, Oxyma pure (Ethyl cyano (hydroxyimino)acetate), diisopropylcarbodiimide (DIC), N,N-dicyclohexylcarbodiimide (DCC), N,N-diisopropylcarbodiimide, 1-hydroxybenzotriazole (HOBt), 1-hydroxy-7-azabenzotriazole (HOAt), benzotriazol-1-yl-oxy-tris(dimethylamino)phosphoniumhexafluorophosphate (BOP), benzotriazol-1-yl-oxy-tris(pyrrolidino)phosphoniumhexafluorophosphate (PyBOP), 7-azabenzotriazol-1-yl-oxytris(pyrrolidino)phosphonium hexafluorophosphate (PyAOP), O-benzotriazol-1-yl-N,N,N′,N′-tetramethyluronium hexafluorophosphate (HBTU), O-(7-azabenzotriazol-1-yl)-N,N, N′N′-tetramethyluronium hexafluorophosphate (HATU), O-(6-Chlorobenzotriazol-1-yl)-N, N,N′,N′-tetramethyluronium hexafluorophosphate (HCTU), O-benzotriazol-1-yl-N,N,N′,N′-tetramethyluronium tetrafluoroborate (TBTU), O-(3,4-dihydro-4-oxo-1,2,3-benzotriazine-3-yl)-N, N,N′,N′-tetramethyluronium tetrafluoroborate (TDBTU), 3-(diethylphosphoryloxy)-1,2,3-benzotriazin-4 (3H)-one (DEPBT), ethyl-(N′,N′-dimethylamino) propylcarbodiimide hydrochloride (EDC), 4, 4, 4-trifluoro-N-Fmoc-O-tert-butyl-threonine.

Typically, secondary binding sites provided by the insoluble support comprise a cleavable linker enabling the cleavage of the target peptide from the insoluble support. Useful linkers include Rink, PAL, Ramage, Sieber, MBHA (methylbenzylhydryl amine), PAM, Wang (4-hydroxybenzyl alcohol moiety), Trityl, 2-chlorotrityl, oxime, HMPA (hydroxymethyl benzoic acid), DHP, Weinreb aminomethyl.

Useful solvents for the preparation of the insoluble support are methyl chloride, N-methylpyrrolidone (NMP), N-methyl-2-pyrrolidone (NMP) and dimethylformamide (DMF).

### Peptide Synthesis Using the Insoluble Support of the Invention

The insoluble support of the invention is preferably applied in the synthesis of peptides. According to an aspect, the peptide synthesis is initiated after the formation of the insoluble support. Thus, a peptide synthesis protocol may start with the provision of a polymeric matrix and subsequent synthesis of the construct thereby forming the insoluble support of the invention. Prior to initiation of the target peptide synthesis the insoluble support may be subjected to treatment steps bringing the insoluble support in a condition suited for subsequent target peptide synthesis.

Any solid phase peptide synthesis chemistry adapted for the synthesis of the modified insoluble support may be applied for the synthesis of the target peptide including a variety of amine protection and side chain protection chemistries and peptide bond formation activation chemistries.

According to an aspect, the insoluble support is specifically suited for the synthesis of long, complex peptides, i.e. peptides having more than 10, 15, 20, 25, 30 amino acids and even peptides with more than 50 amino acids.

The insoluble supports of the invention are capable of synthesizing complex peptides at commercially acceptable yields at commercially useful purity.

The insoluble supports of the invention are typically formed using commercially available homogeneous polymeric matrices/resins which are modified by synthesizing a branched construct from the available primary binding sites of a polymeric matrix.

Thus, the insoluble supports based on commercially available homogeneous polymeric resins is a commercially attractive option to successfully synthesis complex peptides without developing new sophisticated resins. A commercially available homogeneous polymeric resin can by a fairly simple, non-complex SPPS scheme, and the application of readily available amino acids, be transformed into an insoluble support which is capable of generating complex peptides in SPPS at high yields and at commercially relevant purities, and at high throughput.

The present invention also relates to a method for synthesizing peptides having at least 10, at least 15, at least 20, at least 25, at least 30, at least 35, at least 40 amino acids using SPPS and the application of the insoluble support disclosed herein.

The general process for synthesizing peptides on a resin starts by attaching the first amino acid, the C-terminal residue, to the resin. To prevent the polymerization of the amino acid, the alpha amino group and the reactive side chains are protected with a temporary protecting group. Once the amino acid is attached to the resin, the resin is filtered and washed to remove byproducts and excess reagents. Next, the N-alpha protecting group is removed in a deprotection process and the resin is again washed to remove byproducts and excess reagents. Then the next amino acid is coupled to the attached amino acid. This is followed by another washing procedure, which leaves the resin-peptide ready for the next coupling cycle. The cycle is repeated until the peptide sequence is complete. Then typically, the peptide is cleaved off from the resin together with the removal of all the protecting groups. Alternatively, the side chain protecting groups are removed, the peptide resin is washed, and the peptide is cleaved from the resin.

Typically, an N-α-carbamoyl protected amino acid and the N-terminal amino acid on the growing peptide chain attached to a resin with a linker construct are coupled at room temperature in an inert solvent such as dimethylformamide in the presence of coupling agents such as diisopropyl-carbodiimide and Oxyma pure. After amide bond formation the Nα-carbamoyl protecting group is removed from the resulting peptide resin using a reagent such as piperidine, and the coupling reaction is repeated with the next desired Nα-protected amino acid to be added to the peptide chain.

Suitable amine protecting groups are well known in the art and are described, for example, in Green and Wuts, “Protecting Groups in Organic Synthesis,” John Wiley and Sons, 1991. The most commonly used examples include fluorenylmethoxycarbonyl (Fmoc) and Boc (tert-butyloxycarbonyl). After completion of the synthesis, peptides are cleaved from the solid-phase support preferably with simultaneous side-chain deprotection using standard treatment methods under acidic conditions.

The target crude peptide is cleaved off from the insoluble support comprising the construct. The target peptide cleavage conditions are in part dependent on the linker used and type of side chain protection groups.

Crude peptides are typically analyzed using RP-HPLC on C18 (CHS-Agilent UPLC) columns using water-acetonitrile gradients in 0.1% TFA. Purity can be verified by analytical RPHPLC. The identity of peptides can be verified by mass spectrometry. Peptides can be solubilized in aqueous buffers over a wide pH range.

The insoluble support of the invention may be re-generated after cleavage of the target peptide. The insoluble support may be regenerated after separation of the target peptide from the peptide insoluble support solution . . .

### DETAILED DISCLOSURE OF CERTAIN ASPECT OF THE INVENTION

Hereinafter is disclosed insoluble supports comprising constructs proving two 2×, four 4×, seven 7×, eight 8× and sixteen-fold 16× increase of the initial (primary) binding sites of the polymeric matrix. Lysine (K or Lys) is used throughout as the branching agent. Where applicable glycine (G or Gly), polyethylene glycol (PEG₆) or beta-alanine (βAla) are used as a spacer. PEG₆ denotes that the PEG contains six individual PEG moieties covalently bound by peptide bonds.

Aminomethyl (AMS) polystyrene resins are used as the starting polymeric matrix for the synthesis of all insoluble supports presented below. All AMS resins used are amino methyl modified polystyrene resin cross-linked with 1% divinylbenzene. The AMS resins are provided in beads in the 35-150 μm (100 to 200 mesh) range with a degree of substitution of 0.6, 0.93 and 1.95 mmol/gram, respectively

As Lysine is used as the branching agent, the constructs of the disclosed insoluble supports in this section can be denoted as branched polypeptides synthesized on a polymeric matrix.

For all insoluble supports lysine in the S-configuration (L amino acid) and R-configuration (D amino acid) has been used.

For all insoluble supports (2×(Gly), 4×(Gly), 8×(Gly), 16×(Gly)) except 7× linear the branching agent lysine is provided with two Fmoc groups for the protection of the two amines, i.e. the branching agent is provided as Fmoc-Lys(Fmoc)-OH.

The 7× linear is a linear construct. In the synthesis of the linear 7× insoluble support two classes of lysine have been used differing in terms of amine protection groups. One class of lysines has both amine groups protected by Fmoc: Fmoc-Lys(Fmoc)-OH. The other class of lysines have the alpha amine protected by Fmoc and the epsilon amine protected by Mtt: Fmoc-Lys(Mtt)-OH.

### Presentation of the Structures of Some of the Reactants:

Structural formulas of Fmoc-Lys(Fmoc) (R and S configuration) where both amine groups are Fmoc protected:

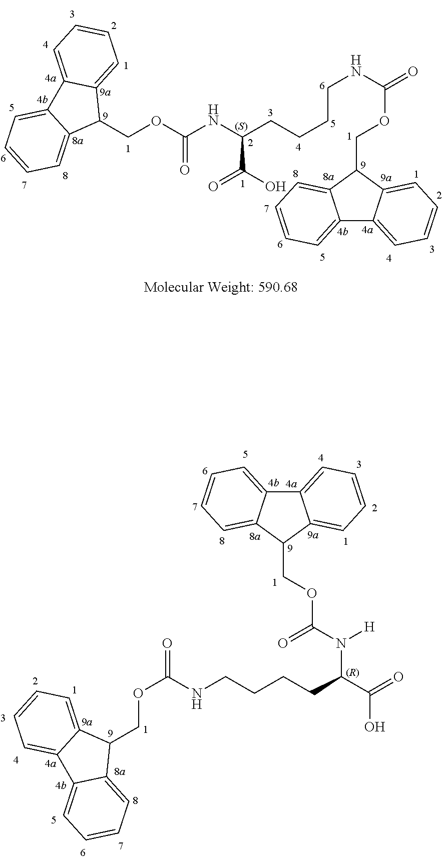

Structural formulas of Fmoc-Lys(Mtt)-OH (S configuration) where the N-terminal, alpha amine is Fmoc protected and the side chain, epsilon amine group is Mtt or Mmt protected.

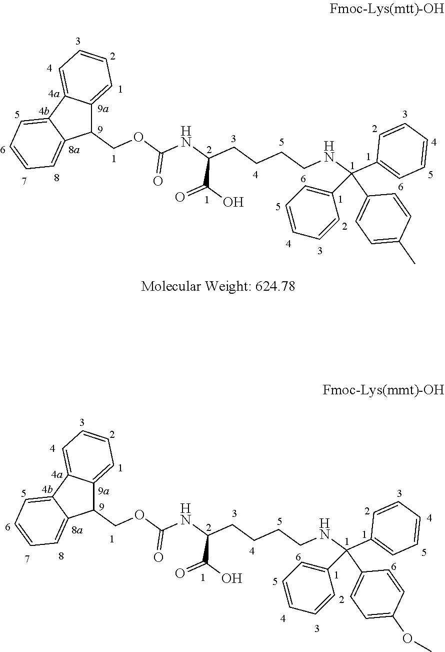 [SMILES: Cc1ccc(C(NCCCC[C@H](NC(=O)OCC2c3ccccc3-c3ccccc32)C(=O)O)(c2ccccc2)c2ccccc2)cc1 ; COc1ccc(C(NCCCC[C@H](NC(=O)OCC2c3ccccc3-c3ccccc32)C(=O)O)(c2ccccc2)c2ccccc2)cc1]

Fmoc-Rink Amide linker (Fmoc-Rink-OH) has the following chemical structure:

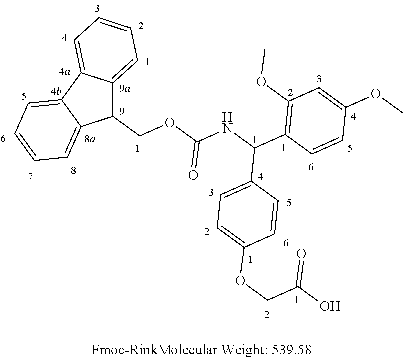 [SMILES: COc1ccc(C(NC(=O)OCC2c3ccccc3-c3ccccc32)c2ccc(OCC(=O)O)cc2)c(OC)c1]

### Fmoc Ramage Linker (MW: 505.58):

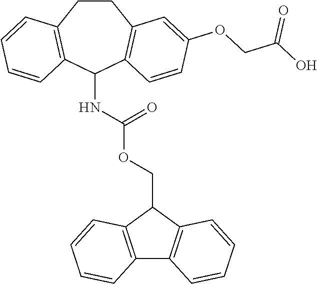 [SMILES: O=C(O)COc1ccc2c(c1)CCc1ccccc1C2NC(=O)OCC1c2ccccc2-c2ccccc21]

### HMPB TBDMS and HMPB Linkers:

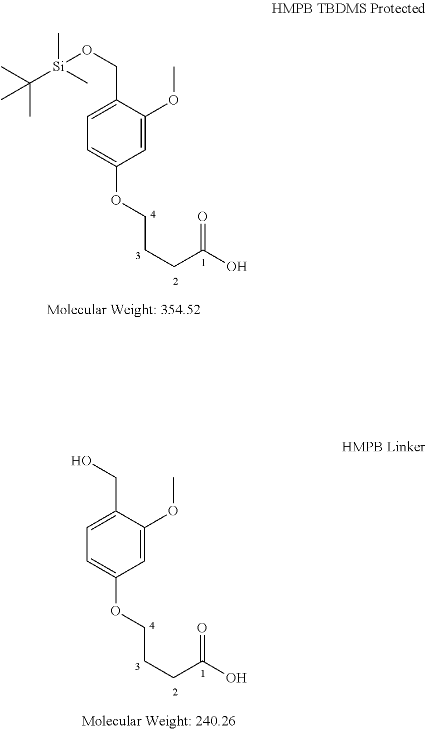 [SMILES: COc1cc(OCCCC(=O)O)ccc1CO[Si](C)(C)C(C)(C)C ; COc1cc(OCCCC(=O)O)ccc1CO]

The glycine spacer is provided as an Fmoc protected glycine. The PEG is provided as 2-[2-[2-(Fmoc amino) ethoxy]ethoxy] acetic acid (EAAE).

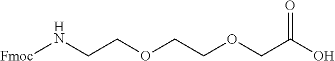

Comments to the nomenclatures of the exemplified insoluble supports:

The number in parenthesis with respect to the branching agent lysine K (or Lys) denotes the layer or generation whereas the subscript denotes the total number of lysines of each layer/generation. E.g. K(2)₂ or Lys(2)₂ denotes that the construct of the insoluble support has two secondary lysines.

### Disclosure of Solid Supports

Solids supports 2×(Gly), 4×(Gly) and 8×(Gly) based on a 0.60 mmol/g AMS polymeric matrix (without linkers) were synthesized using the following protocol:

Starting AMS resin with an initial loading of 0.60 mmol/g (30.0 g, table 1) was introduced into 1 L glass reactor equipped with mechanical stirrer and swollen in DMF (8 mL/g of starting resin, 2×1 h). All amino acid couplings were carried out using a molar ratio of 1:1:1.25 of Fmoc amino acid (4.0 eq.) in DMF (0.50M), Oxyma (4.0 eq.) and N′N′-Diisopropylcarbodiimide (5.0 eq.). Fmoc amino acids (Fmoc-Gly-OH or Fmoc-Lys(Fmoc)-OH) were dissolved in DMF (0.50M) followed by the addition of Oxyma and N′N′-Diisopropylcarbodiimide and pre-activated for 30 min at room temperature before being transferred to the glass reactor. Couplings were carried out for 2 h at room temperature. Following coupling, the resin was washed with DMF (2×8 mL/g), capped using DMF/acetic anhydride (Ac₂O)/Diisopropylethylamine (DIEA) (1:0.04:0.09) for 30 min and re-washed using DMF (7×8 mL/g). Fmoc groups were deprotected prior to each coupling step using 20% piperidine in DMF (1×10 min+1×20 min) at room temperature. Following Fmoc-deprotection, the resin was washed using DMF (7×8 mL/g of starting resin).

Following the completion of the 2×-Gly-AMS synthesis, the resin was washed with CH₂Cl₂ (3×), iPrOH (3×) and MTBE (3×) prior to its drying under vacuum overnight. ⅓ of the obtained resin was conserved while the other ⅔ (28.2 g, table 1) were used for the synthesis of 4×-Gly-AMS in the same way as 2×-Gly-AMS.

Following the completion of the 4×-Gly-AMS synthesis, the resin was washed with CH₂Cl₂ (3×), iPrOH (3×) and MTBE (3×) prior to its drying under vacuum overnight. ⅔ of the obtained resin was conserved while the other ⅓ was used for the synthesis of 8×-Gly-AMS in the same way as 2×-Gly-AMS.

Following the completion of the 8×-Gly-AMS synthesis, the resin was washed with CH₂Cl₂ (3×), iPrOH (3×) and MTBE (3×) prior to its drying under vacuum overnight.

TABLE 1

| | Mass of | | |
|---|---|---|---|
| | starting | | Mass of |
| | polymeric | Mass of | modified |
| | matrix | modified | polymeric |
| | (AMS 0.60 | polymeric | matrix |
| | mmol/g) | matrix | (insoluble |
| Type of | of each | (insoluble | support) |
| insoluble | synthesis | support) | conserved |
| support | [g] | [g] | [g] |
| 2X(Gly)-AMS 0.6 | 30.0 of AMS-0.6 | 42.30 | 14.10 |
| 4X(Gly)-AMS 0.6 | 28.2 of 2X(Gly)-AMS-0.6 | 42.60 | 28.40 |
| 8X(Gly)-AMS 0.6 | 14.2 of 4X(Gly)-AMS 0.6 | 20.38 | 20.38 |

Solids supports 2×(Gly) and 4×(Gly) based on a 0.93 mmol/g AMS polymeric matrix and 1.95 mmol/g AMS polymeric matrix respectively (without linkers) were synthesized using the following protocol:

Starting AMS resin with an initial loading of 0.93 or 1.95 mmol/g (1.0 or 2.0, tables 2 and 3) was introduced into 60 mL plastic syringes and swollen in DMF (8 mL/g of starting resin, 2×1 h). Syntheses were carried out manually where syringes were shaken with an Activo-PLS 4×4 synthesizer purchased from Activotec at 400 rpm in horizontal position. All amino acid couplings were carried out using a molar ratio of 1:1:1.25 of Fmoc amino acid (4.0 eq.) in DMF (0.50M), Oxyma (4.0 eq.) and N′N′-Diisopropylcarbodiimide (5.0 eq.). Fmoc amino acids (Fmoc-Gly-OH or Fmoc-Lys (Fmoc)-OH) were dissolved in DMF (0.50M) followed by the addition of Oxyma and N′N′-Diisopropylcarbodiimide and pre-activated for 30 min at room temperature before being transferred to the glass reactor. Couplings were carried out for 2 h at room temperature. Following coupling, the resin was washed with DMF (2×8 mL/g), capped using DMF/acetic anhydride (Ac₂O)/Diisopropylethylamine (DIEA) (1:0.04:0.09) for 30 min and re-washed using DMF (7×8 mL/g). Fmoc groups were deprotected prior to each coupling step using 20% piperidine in DMF (1×10 min+1×20 min) at room temperature. Following Fmoc-deprotection, the resin was washed using DMF (7×8 mL/g of starting resin).

2×-Gly-AMS and 4×-Gly-AMS soluble supports synthesized using AMS resin of 0.93 mmol/g were isolated starting from the same reactor batch of starting AMS resin (2.0 g). Following the completion of the 2×-Gly-AMS synthesis, the resin was washed with CH₂Cl₂ (3×), iPrOH (3×) and MTBE (3×) prior to its drying under vacuum overnight. ½ of the obtained resin was conserved while the other ½ was used for the synthesis of 4×-Gly-AMS in the same way as 2×-Gly-AMS. Following the completion of the 4×-Gly-AMS synthesis, the resin was washed with CH₂Cl₂ (3×), iPrOH (3×) and MTBE (3×) prior to its drying under vacuum overnight.

2×-Gly-AMS and 4×-Gly-AMS soluble supports synthesized using AMS resin of 1.95 mmol/g were isolated starting from starting AMS resin batches of 1.0 g and 2.0 g, respectively. Following the completion of their synthesis, resins were washed with CH₂Cl₂ (3×), iPrOH (3×) and MTBE (3×) prior to their drying under vacuum overnight.

TABLE 2

| | Mass of | | Mass of |
|---|---|---|---|
| | starting | Mass of | modified |
| | polymeric | modified | polymeric |
| | matrix | polymeric | matrix |
| | (AMS 0.93 | matrix | (insoluble |
| Type of | mmol/g) of | (insoluble | support) |
| insoluble | each | support) | conserved |
| support | synthesis [g] | [g] | [g] |
| 2X(Gly)-AMS 0.93 | 2.0 of AMS 0.93 | 3.52 | 1.76 |
| 4X(Gly)-AMS 0.93 | 1.76 of 2X(Gly)-AMS 0.93 | 2.67 | 2.67 |

TABLE 3

| | Mass of | | Mass of |
|---|---|---|---|
| | starting | Mass of | modified |
| | polymeric | modified | polymeric |
| | matrix | polymeric | matrix |
| | (AMS 1.95 | matrix | (insoluble |
| Type of | mmol/g) | (insoluble | support) |
| insoluble | of each | support) | conserved |
| support | synthesis [g] | [g] | [g] |
| 2X(Gly)-AMS 1.95 | 1.0 of AMS 1.95 | 2.40 | 2.40 |
| 4X(Gly)-AMS 1.95 | 2.0 of AMS 1.95 | 7.80 | 7.80 |

### Synthesis Protocols of Different Fmoc-Rink Amide Insoluble Supports

2×(Gly): (Fmoc-Rink)₂-Lys(1)-Gly-AMS
4×(Gly): (Fmoc-Rink)₄-Lys(2)₂-Gly₂-Lys(1)-Gly-AMS
8×(Gly): (Fmoc-Rink)₈-Lys(3)₄-Gly₄-Lys(2)₂-Gly₂-Lys(1)-Gly-AMS

Relevant soluble supports presented above were modified by the coupling of Rink amide using the following protocol:

Starting resins (1.0 g each) were introduced into 25 mL syringes and swollen in DMF (6.5 mL/g, 2×1 h). Syntheses were carried out manually where syringes were shaken with an Activo-PLS 4×4 synthesizer purchased from Activotec at 400 rpm in horizontal position. Fmoc groups were deprotected prior to rink amide linker coupling using 20% piperidine in DMF (1×10 min+1×20 min) at room temperature. Following Fmoc-deprotection, resins were washed using DMF (7×6.5 mL/g of starting resin). All rink amide couplings were carried out using 1:1:2 molar ratio of Rink amide linker in DMF (0.50M), PyAOP and Diisopropylethylamine (DIEA) at a 5-fold molar excess compared to synthesis scale. Rink amide was dissolved in DMF (0.50M) followed by the addition of PyAOP and stirred for 10 min at room temperature before adding DIEA. Next, the solution was introduced into the resin-containing syringes. Couplings were carried out overnight at room temperature. Following coupling, resins were washed with DMF (2×8 mL/g) and capped using DMF/acetic anhydride (Ac₂O)/Diisopropylethylamine (DIEA) (1:0.04:0.09) for 30 min. Next, resins were washed using DMF (7×8 mL/g), isopropanol (iPrOH, 3×) and methyl tert-butyl ether (MTBE, 3×) prior to their drying under vacuum overnight.

Table 4 presents the resin substitution before and after Rink amide coupling.

TABLE 4

| Base resin/ | | Resin | Resin |
|---|---|---|---|
| polymeric | | substitution | substitution |
| matrix | Resin type/ | before Rink | after Rink |
| [substitution | insoluble | amide coupling | amide coupling |
| in mmol/g] | support | [mmol/g] | [mmol/g] |
| AMS (0.60) | AMS | 0.60 | 0.48 |
| AMS (0.60) | 2X(Gly)-AMS | 0.90 | 0.68 |
| AMS (0.60) | 4X(Gly)-AMS | 1.20 | 0.90 |
| AMS (0.60) | 8X(Gly)-AMS | 1.76 | 0.98 |
| AMS (0.93) | AMS | 0.93 | 0.62 |
| AMS (0.93) | 2X(Gly)-AMS | 1.06 | 0.82 |
| AMS (0.93) | 4X(Gly)-AMS | 1.48 | 1.10 |
| AMS (1.95) | AMS | 1.95 | 1.08 |
| AMS (1.95) | 2X(Gly)-AMS | 1.56 | 1.14 |
| AMS (1.95) | 4X(Gly)-AMS | 1.80 | 1.20 |

### Synthesis Protocols of Different Fmoc-Ramage Insoluble Supports

2×(Gly): (Fmoc-Ramage)₂-Lys(1)-Gly-AMS
4×(Gly): (Fmoc-Ramage)₄-Lys(2)₂-Gly₂-Lys(1)-Gly-AMS
8×(Gly): (Fmoc-Ramage)₈-Lys(3)₄-Gly₄-Lys(2)₂-Gly₂-Lys(1)-Gly-AMS

Relevant soluble supports presented above were modified by the coupling of Ramage using the following protocol:

Starting resins (1.0 g each) were introduced into 25 mL syringes and swollen in DMF (6.5 mL/g, 2×1 h). Syntheses were carried out manually where syringes were shaken with an Activo-PLS 4×4 synthesizer purchased from Activotec at 400 rpm in horizontal position. Fmoc groups were deprotected prior to Ramage linker coupling using 20% piperidine in DMF (1×10 min+1×20 min) at room temperature. Following Fmoc-deprotection, resins were washed using DMF (7×6.5 mL/g of starting resin). All ramage couplings were carried out using an equimolar ratio 1:1:1 of Ramage linker in DMF (0.15M-0.40M), Oxyma and N′N′-Diisopropylcarbodiimide (DIC) at a 2-fold molar excess compared to synthesis scale. Ramage was dissolved in DMF (0.15M-0.40M) followed by the addition of Oxyma and stirred for 10 min at room temperature before adding DIC. Next, the solution was introduced into the resin-containing syringes. Couplings were carried out overnight at room temperature. Following coupling, resins were washed with DMF (2×8 mL/g) and capped using DMF/acetic anhydride (Ac₂O)/Diisopropylethylamine (DIEA) (1:0.04:0.09) for 30 min. Next, resins were washed using DMF (7×8 mL/g), isopropanol (iPrOH, 3×) and methyl tert-butyl ether (MTBE, 3×) prior to their drying under vacuum overnight.

Table 5 presents the resin substitution before and after Ramage coupling.

TABLE 5

| Base resin/ | | Resin | Resin |
|---|---|---|---|
| polymeric | | substitution | substitution |
| matrix | Resin type/ | before Rink | after Rink |
| [substitution | insoluble | amide coupling | amide coupling |
| in mmol/g] | support | [mmol/g] | [mmol/g] |
| AMS (0.60) | AMS | 0.60 | 0.48 |
| AMS (0.60) | 2X(Gly)-AMS | 0.90 | 0.62 |
| AMS (0.60) | 4X(Gly)-AMS | 1.20 | 1.12 |
| AMS (0.60) | 8X(Gly)-AMS | 1.76 | 1.16 |
| AMS (0.93) | AMS | 0.93 | 0.49 |
| AMS (0.93) | 2X(Gly)-AMS | 1.06 | 1.00 |
| AMS (0.93) | 4X(Gly)-AMS | 1.48 | 1.04 |
| AMS (1.95) | AMS | 1.95 | 0.82 |
| AMS (1.95) | 2X(Gly)-AMS | 1.56 | 0.94 |
| AMS (1.95) | 4X(Gly)-AMS | 1.80 | 0.90 |

### Synthesis Protocols of Different Fmoc-Leu-HMPB Insoluble Supports

4×(Gly): (Fmoc-Leu-HMPB-Leu)₄-Lys(2)₂-Gly₂-Lys(1)-Gly-AMS
8×(Gly): (Fmoc-Leu-HMPB-Leu)₈-Lys(3)₄-Gly₄-Lys(2)₂-Gly₂-Lys(1)-Gly-AMS Relevant soluble supports presented above were modified by the coupling Fmoc-Leu-HMPB using the following protocol:

Starting resins (1.0 g each) were introduced into 25 mL syringes and swollen in DMF (6.5 mL/g, 2×1 h). Syntheses were carried out manually where syringes were shaken with an Activo-PLS 4×4 synthesizer purchased from Activotec at 400 rpm in horizontal position. Fmoc groups were deprotected prior to TBDMS-HMPB coupling using 20% piperidine in DMF (1×10 min+1×20 min) at room temperature. Following Fmoc-deprotection, resins were washed using DMF (7×6.5 mL/g of starting resin). AII TBDMS-HMPB couplings were carried out using 1:1:2 molar ratio of TBDMS-HMPB in DMF (0.50M), PyOxim and Diisopropylethylamine (DIEA) at a 2-fold molar excess compared to synthesis scale. TBDMS-HMPB was dissolved in DMF (0.50M) followed by the addition of PyOxim and stirred for 10 min at room temperature before adding DIEA. Next, the solution was introduced into the resin-containing syringes. Couplings were carried out overnight at room temperature. Following HMPB coupling, resins were washed with DMF (2×8 mL/g) and capped using DMF/acetic anhydride (Ac₂O)/Diisopropylethylamine (DIEA) (1:0.04:0.09) for 30 min. Next, resins were washed using DMF (7×8 mL/g), isopropanol (iPrOH, 3×) and methyl tert-butyl ether (MTBE, 3×) prior to their drying under vacuum overnight.

Starting HMPB-resins (300 mg each) were introduced into 12 mL syringes and swollen in THF (6.5 mL/g, 2×1 h). Syntheses were carried out manually where syringes were shaken with an Activo-PLS 4×4 synthesizer purchased from Activotec at 400 rpm in horizontal position. TBDMS group was deprotected prior to Fmoc-Leu-OH coupling using Et3N.3HF (12 equiv. compared to synthesis scale) in THF (0.22M) for 2 h at room temperature. Following TBDMS-deprotection, resins were washed using DIEA (1×) then using DMF until neutral PH.

Prior to their coupling with Fmoc-Leu-OH, resins were swollen in DMF (6.5 mL/g, 2×1 h). All couplings were carried out using 1:1:1:0.25 molar ratio of Fmoc-Leu-OH in DMF (0.45M), Oxyma, N′N′-Diisopropylcarbodiimide (DIC) and 4-Dimethylaminopyridine (DMAP) at a 2.4-fold molar excess compared to synthesis scale. Fmoc-Leu-OH was dissolved in DMF (0.45M) followed by the addition of Oxyma, DIC and DMAP and stirred for 5 min at room temperature before being introduced into the resin-containing syringes. Couplings were carried out overnight at room temperature. Following coupling, resins were washed with DMF (2×8 mL/g) and capped using DMF/acetic anhydride (Ac₂O)/Diisopropylethylamine (DIEA) (1:0.04:0.09) for 30 min. Next, resins were washed using DMF (7×8 mL/g), isopropanol (iPrOH, 3×) and methyl tert-butyl ether (MTBE, 3×) prior to their drying under vacuum overnight.

Table 6 presents the resin substitution before and after Leu-HMPB coupling.

TABLE 6

| Base resin/ | | Resin | Resin |
|---|---|---|---|
| polymeric | | substitution | substitution |
| matrix | Resin type/ | before Rink | after Rink |
| [substitution | insoluble | amide coupling | amide coupling |
| in mmol/g] | support | [mmol/g] | [mmol/g] |
| AMS (0.60) | 4X(Gly)-AMS | 1.20 | 0.88 |
| AMS (0.60) | 8X(Gly)-AMS | 1.76 | 0.90 |
| AMS (0.93) | AMS | 0.93 | 0.49 |

### Synthesis Protocols of Different Fmoc-Gly-HMPB Insoluble Supports

4×(Gly): (Fmoc-Gly-HMPB)₄-Lys(2)₂-Gly₂-Lys(1)-Gly-AMS
8×(Gly): (Fmoc-Gly-HMPB-)₈-Lys(3)₄-Gly₄-Lys(2)₂-Gly₂-Lys(1)-Gly-AMS

Relevant soluble supports presented above were modified by the coupling Fmoc-Gly-HMPB using the following protocol:

Starting resins (1.0 g each) were introduced into 25 mL syringes and swollen in DMF (6.5 mL/g, 2×1 h). Syntheses were carried out manually where syringes were shaken with an Activo-PLS 4×4 synthesizer purchased from Activotec at 400 rpm in horizontal position. Fmoc groups were deprotected prior to TBDMS-HMPB coupling using 20% piperidine in DMF (1×10 min+1×20 min) at room temperature. Following Fmoc-deprotection, resins were washed using DMF (7×6.5 mL/g of starting resin). All TBDMS-HMPB couplings were carried out using 1:1:2 molar ratio of TBDMS-HMPB in DMF (0.50M), PyOxim and Diisopropylethylamine (DIEA) at a 2-fold molar excess compared to synthesis scale. TBDMS-HMPB was dissolved in DMF (0.50M) followed by the addition of PyOxim and stirred for 10 min at room temperature before adding DIEA. Next, the solution was introduced into the resin-containing syringes. Couplings were carried out overnight at room temperature. Following HMPB coupling, resins were washed with DMF (2×8 mL/g) and capped using DMF/acetic anhydride (Ac₂O)/Diisopropylethylamine (DIEA) (1:0.04:0.09) for 30 min. Next, resins were washed using DMF (7×8 mL/g), isopropanol (iPrOH, 3×) and methyl tert-butyl ether (MTBE, 3×) prior to their drying under vacuum overnight.

Starting HMPB-resins (300 mg each) were introduced into 12 mL syringes and swollen in THF (6.5 mL/g, 2×1 h). Syntheses were carried out manually where syringes were shaken with an Activo-PLS 4×4 synthesizer purchased from Activotec at 400 rpm in horizontal position. TBDMS group was deprotected prior to Fmoc-Gly-OH coupling using Et3N.3HF (12 equiv. compared to synthesis scale) in THF (0.22M) for 2 h at room temperature. Following TBDMS-deprotection, resins were washed using DIEA (1×) then using DMF until neutral PH.

Prior to their coupling with Fmoc-Gly-OH, resins were swollen in DMF (6.5 mL/g, 2×1 h). All couplings were carried out using 1:1:1:0.25 molar ratio of Fmoc-Gly-OH in DMF (0.45M), Oxyma, N′N′-Diisopropylcarbodiimide (DIC) and 4-Dimethylaminopyridine (DMAP) at a 2.4-fold molar excess compared to synthesis scale. Fmoc-Gly-OH was dissolved in DMF (0.45M) followed by the addition of Oxyma, DIC and DMAP and stirred for 5 min at room temperature before being introduced into the resin-containing syringes. Couplings were carried out overnight at room temperature. Following coupling, resins were washed with DMF (2×8 ml/g) and capped using DMF/acetic anhydride (Ac₂O)/Diisopropylethylamine (DIEA) (1:0.04:0.09) for 30 min. Next, resins were washed using DMF (7×8 ml/g), isopropanol (IPrOH, 3×) and methyl tert-butyl ether (MTBE, 3×) prior to their drying under vacuum overnight.

Table 7 presents the resin substitution before and after Gly-HMPB coupling.

TABLE 7

| Base resin/ | | Resin | Resin |
|---|---|---|---|
| polymeric | | substitution | substitution |
| matrix | Resin type/ | before Rink | after Rink |
| [substitution | insoluble | amide coupling | amide coupling |
| in mmol/g] | support | [mmol/g] | [mmol/g] |
| AMS (0.60) | AMS | 0.60 | 0.40 |
| AMS (0.60) | 4X(Gly)-AMS | 1.20 | 0.88 |
| AMS (0.60) | 8X(Gly)-AMS | 1.76 | 1.00 |
| AMS (0.93) | AMS | 0.93 | 0.53 |

### (Fmoc-Linker)₂-Lys(1)-Gly-AMS Insoluble Support [2×(Gly)]

Linkers: Fmoc-Rink and Fmoc-Ramage
   Molecular Weight of the Respective Constructs without Support:
Fmoc-Rink: Molecular weight (g/mol): 1245.40
Fmoc-Ramage: Molecular weight (g/mol): 1291.47

The constructs of the insoluble supports comprise one lysine, one glycine spacer and two Fmoc-Rink-linkers or two Fmoc-Ramage-linkers coupled to the amines of the first generation (first layer) of lysine, also referred to as distal lysine (distal branching agent). The glycine spacer is positioned between the polymeric matrix and the lysine branching agent.

The (Fmoc-Rink)₂-Lys(1)-Gly and (Fmoc-Ramage)₂-Lys(1)-Gly constructs contain one layer (first generation) of lysine, a primary layer, i.e. primary lysine. Each construct provides two distal binding sites available for peptide synthesis. Two Rink-linkers or two Ramage-linkers are bound to the amine groups (N-terminal alpha amine and side chain epsilon amine) of each lysine. The lysine is bound to the glycine and the glycine bound to the amines of the polymeric matrix.

Only lysine has been used where both amine groups of lysine are Fmoc protected.

The polymeric matrices used for modification are AMS 0.6, AMS 0.93 and AMS 1.95.

Reactants: Fmoc-Lys(Fmoc)-OH, Fmoc-Gly-OH, Fmoc-Rink-OH, Fmoc-Ramage-OH

### Structural Formula of the (Fmoc-Rink)₂-Lys(1)-Gly Construct

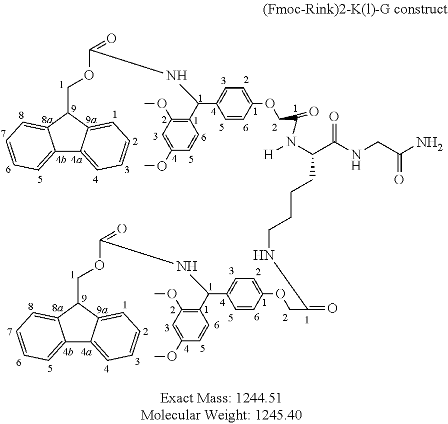

### Structural Formula of the (Fmoc-Ramage)₂-Lys(1)-Gly Construct

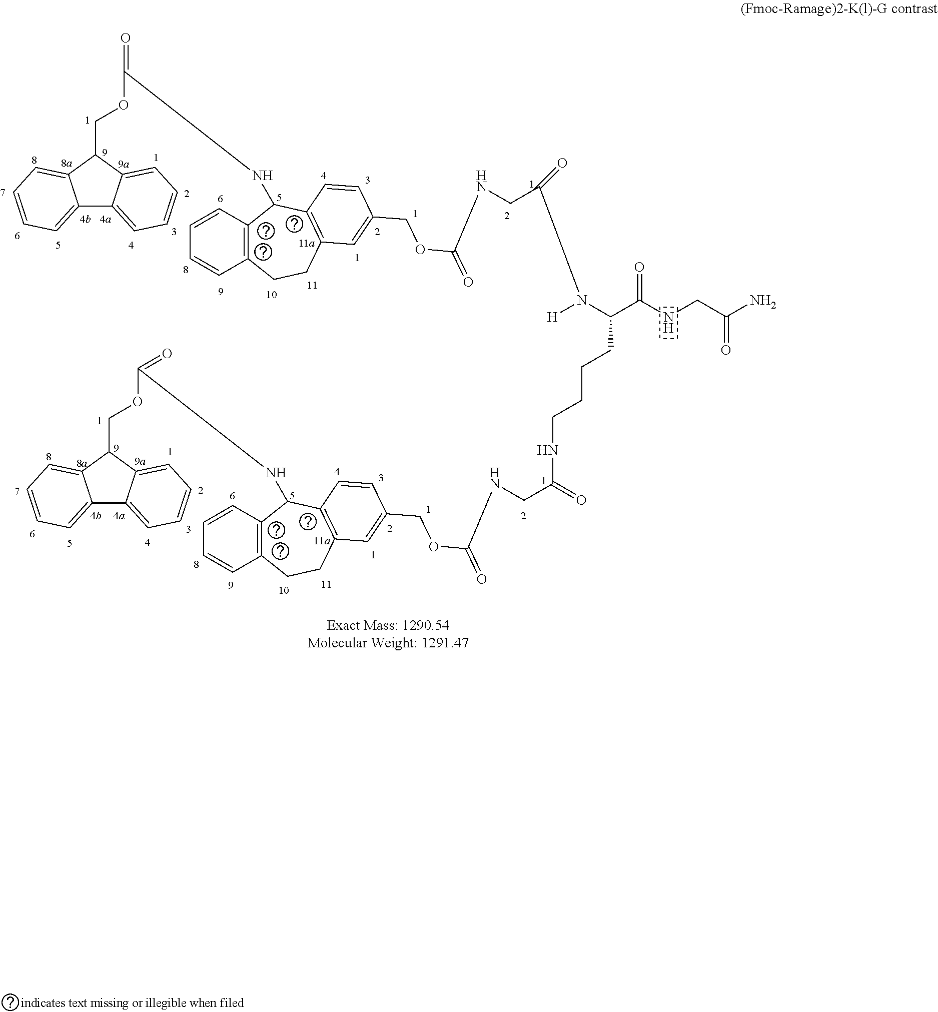 [SMILES: NC(=O)CNC(=O)[C@H](CCCCNC(=O)CNC(=O)OCc1ccc2c(c1)CCc1ccccc1C2NC(=O)OCC1c2ccccc2-c2ccccc21)NC(=O)CNC(=O)OCc1ccc2c(c1)CCc1ccccc1C2NC(=O)OCC1c2ccccc2-c2ccccc21]

### Synthesis Scheme for (Fmoc-Linker)₂-Lys(1)-Gly-AMS:

Linkers: Rink and Ramage

Resin-AMS + Fmoc-Gly-OH
↓ Coupling
Resin-AMS-Gly-Fmoc
↓ deprotection Fmoc, coupling Fmoc-Lys(Fmoc)-OH
Resin-AMS-Gly-Lys(Fmoc)-Fmoc
↓ deprotection Fmoc, coupling Fmoc-Linker-OH
Resin-AMS-Gly-Lys(Fmoc-Linker)-Fmoc-Linker
(Resin-AMS-Gly-Lys(1)-(Fmoc-Linker)₂

(Linker)₄-Lys(2)₂-Gly-Lys(1)-Gly-AMS Insoluble Support [4×(Gly)]
Linkers: Fmoc-Rink, Fmoc-Ramage and HMPB TBDMS
   Molecular Weight of the Respective Constructs without Support:
Fmoc-Rink: Molecular weight (g/mol): 2658.99
Fmoc-Ramage: Molecular weight (g/mol): 2522.93
HMPB TBDMS: Molecular weight (g/mol): 1461.66

The construct of these modified resins comprises Gly (G) in addition to three lysines and four linkers coupled to the second generation lysines. The Gly or beta-Ala spacers are positioned between first and second generation of lysines and between the first generation lysines and polymeric matrix.

The (Fmoc-Rink)₄-Gly(2)₂-Gly₂-Lys(1)-Gly, (Fmoc-Ramage)₄-Lys(2)₂-Gly₂-Lys(1)-Gly and (HMPB TBDMS)₄-Lys(2)₂-Gly₂-Lys(1)-Gly constructs contain two layers (generations) of lysines, a primary and secondary layer, i.e. primary and secondary lysines, the secondary layer of lysines being the most distal lysines with respect to the polymeric matrix. The second layer of two secondary lysines provides in total four secondary binding sites available for peptide synthesis. Two Fmoc-Rink-linkers, two Fmoc-Ramage-linkers or two HMPB TBDMS)-linkers are bound to the two amine groups (N-terminal alpha amine and side chain epsilon amine) of each of the secondary lysines. Thus, the constructs have four linkers.

The polymeric matrices used for modification are AMS 0.6, AMS 0.93 and AMS 1.95.

Only lysine has been used where both amine groups of lysine are Fmoc protected.
Reactants: Fmoc-Lys(Fmoc)-OH, Fmoc-Gly-OH, Fmoc-beta-Ala-OH, Fmoc-Rink-OH, Fmoc-Ramage-OH, HMPB TBDMS-OH
   Structural Formula of the (Fmoc-Rink)₄-Lys(2)₂-Gly₂-Lys(1)-Gly Construct:

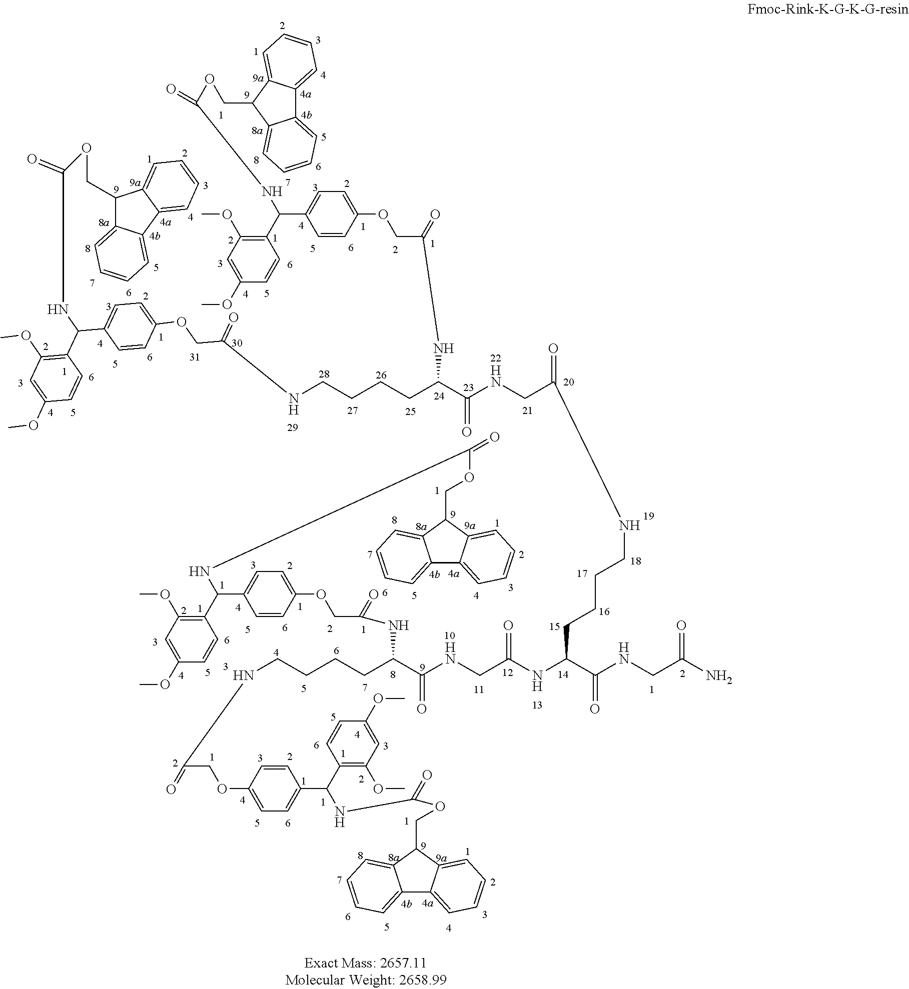 [SMILES: COc1ccc(C(NC(=O)OCC2c3ccccc3-c3ccccc32)c2ccc(OCC(=O)NCCCC[C@H](NC(=O)COc3ccc(C(NC(=O)OCC4c5ccccc5-c5ccccc54)c4ccc(OC)cc4OC)cc3)C(=O)NCC(=O)NCCCC[C@H](NC(=O)CNC(=O)[C@H](CCCCNC(=O)COc3ccc(C(NC(=O)OCC4c5ccccc5-c5ccccc54)c4ccc(OC)cc4OC)cc3)NC(=O)COc3ccc(C(NC(=O)OCC4c5ccccc5-c5ccccc54)c4ccc(OC)cc4OC)cc3)C(=O)NCC(N)=O)cc2)c(OC)c1]

Structural Formula of the (Fmoc-Ramage)₄-Lys(2)₂-Gly₂-Lys(1)-Gly Construct:

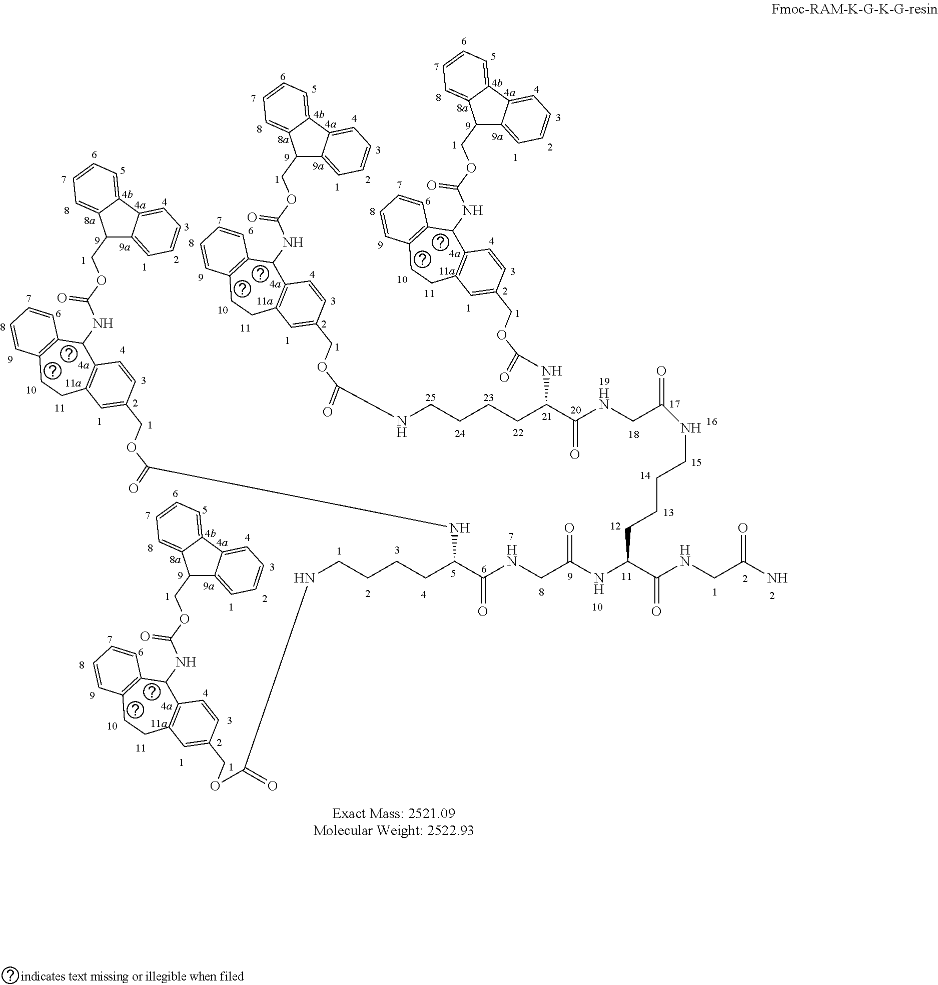 [SMILES: NC(=O)CNC(=O)[C@H](CCCCNC(=O)CNC(=O)[C@H](CCCCNC(=O)OCc1ccc2c(c1)CCc1ccccc1C2NC(=O)OCC1c2ccccc2-c2ccccc21)NC(=O)OCc1ccc2c(c1)CCc1ccccc1C2NC(=O)OCC1c2ccccc2-c2ccccc21)NC(=O)CNC(=O)[C@H](CCCCNC(=O)OCc1ccc2c(c1)CCc1ccccc1C2NC(=O)OCC1c2ccccc2-c2ccccc21)NC(=O)OCc1ccc2c(c1)CCc1ccccc1C2NC(=O)OCC1c2ccccc2-c2ccccc21]

Structural formula of the (TBDMS-HMPB)₄-Lys(2)₂-Gly₂-Lys(1)-Gly construct:

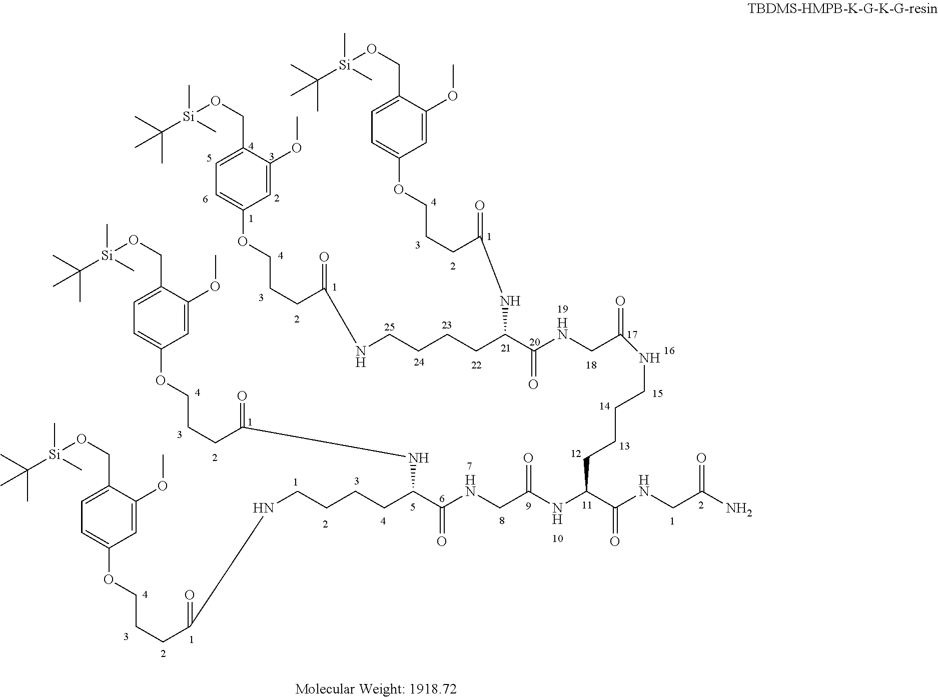 [SMILES: COc1cc(OCCCC(=O)NCCCC[C@H](NC(=O)CCCOc2ccc(CO[Si](C)(C)C(C)(C)C)c(OC)c2)C(=O)NCC(=O)NCCCC[C@H](NC(=O)CNC(=O)[C@H](CCCCNC(=O)CCCOc2ccc(CO[Si](C)(C)C(C)(C)C)c(OC)c2)NC(=O)CCCOc2ccc(CO[Si](C)(C)C(C)(C)C)c(OC)c2)C(=O)NCC(N)=O)ccc1CO[Si](C)(C)C(C)(C)C]

For the characterization of 4× constructs a (NH₂-Rink)₄-Lys(2)₂-Gly₂-Lys(1)-Gly-NH₂ and (NH₂-Rink)₄-Lys(2)₂-βAla₂-Lys(1)-βAla-NH₂ were synthesized using Fmoc chemistry on a Symphony multi-channel peptide synthesizer. In order to cleave the construct from the polymeric matrix a sieber amide resin was used (Cas 915706-90-0). Branching agent Fmoc-Lys(Fmoc)-OH, Oxyma, Dic (Diisopropylcarbodiimide) and spacers (Gly, βAla=Beta alanine) were used at a 3-fold molar excess over the theoretical free amino groups. The amide couplings were allowed to proceed for 3 hours at RT. The obtained constructs were characterized by LC/MC.

(NH₂-Rink)₄-Lys(2)₂-Gly₂-Lys(1)-Gly-NH₂
Calculated mass: 1770.99
Calculated: (M+2H)2+=886.49
Observed: (M+2H)2+=885.48

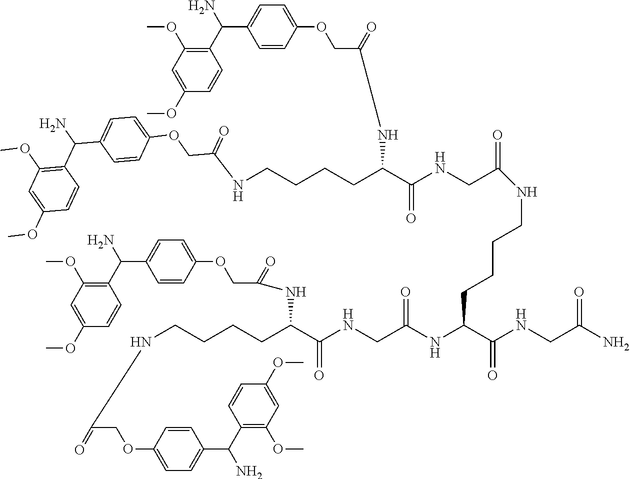 [SMILES: COc1ccc(C(N)c2ccc(OCC(=O)NCCCC[C@H](NC(=O)COc3ccc(C(N)c4ccc(OC)cc4OC)cc3)C(=O)NCC(=O)NCCCC[C@H](NC(=O)CNC(=O)[C@H](CCCCNC(=O)COc3ccc(C(N)c4ccc(OC)cc4OC)cc3)NC(=O)COc3ccc(C(N)c4ccc(OC)cc4OC)cc3)C(=O)NCC(N)=O)cc2)c(OC)c1]

(NH₂-Rink)₄-Lys(2)₂-βAla₂-Lys(1)-βAla-NH₂
Calculated mass: 1810.89
Calculated (M+2H)2+=906.46
Observed: (M+2H)2+=907.36,

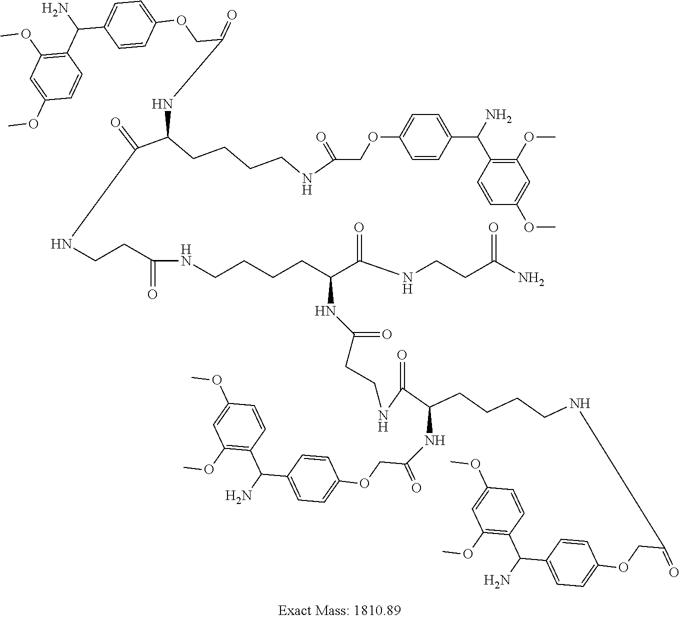 [SMILES: COc1ccc(C(N)c2ccc(OCC(=O)NCCCC[C@H](NC(=O)COc3ccc(C(N)c4ccc(OC)cc4OC)cc3)C(=O)NCCC(=O)NCCCC[C@H](NC(=O)CCNC(=O)[C@@H](CCCCNC(=O)COc3ccc(C(N)c4ccc(OC)cc4OC)cc3)NC(=O)COc3ccc(C(N)c4ccc(OC)cc4OC)cc3)C(=O)NCCC(N)=O)cc2)c(OC)c1]

Synthesis Scheme for (Linker)₄-K(2)₂-G₂-K(1)-G-AMS):
Linkers: Fmoc-Rink, Fmoc-Ramage and HMPB TBDMS

Resin-AMS + Fmoc-Gly-OH
↓ Coupling, deprotection Fmoc
Resin-AMS-Gly-H
↓coupling Fmoc-Lys(Fmoc)-OH
Resin-AMS-Gly-Lys(Fmoc)-Fmoc
↓deprotection Fmoc, coupling Fmoc-Gly-OH
Resin-AMS-Gly-Lys(Gly-Fmoc)-Gly-Fmoc
↓ Deprotection Fmoc, coupling Fmoc-Lys(Fmoc)-OH
Resin-AMS-Gly-Lys(Gly-Lys(Fmoc)-Fmoc)-Gly-
Lys(Fmoc)-Fmoc
↓ deprotection Fmoc, coupling Linker-OH
Resin-AMS-Gly-Lys(Gly-Lys(Linker)-Linker)-Gly-
Lys(Linker)-Linker
Resin-AMS-G-K(1)-G₂-K(2)₂-(Linker)₄

(Linker)₈-Lys(3)₄-Gly₄-Lys(2)₂-Gly₂-K(1)-Gly-AMS Insoluble Support [8×(Gly)]
Linkers: Fmoc-Rink, Fmoc-Ramage and HMPB TBDMS
Fmoc-Rink: Molecular weight (g/mol): 5486.17
Fmoc-Ramage: Molecular weight (g/mol): 4370.12
HMPB TBDMS: Molecular weight (g/mol): 4005.64

The construct of this insoluble support increases each primary binding site of the base AMS resin theoretically 8-fold. The construct comprises seven lysines, seven glycines and eight linkers. The construct has three layers/generations of lysines, one primary (proximal) lysine, two secondary (intermediate) lysines and four tertiary (distal) lysines. All secondary intermediate lysines between the primary (proximal lysine) and distal lysines (here tertiary lysines) are all bound to three different lysines signifying that the construct is symmetrical.

The polymeric matrix used for modification is AMS 0.6.

All lysines are exclusively of the type Fmoc-Lys(Fmoc)-OH.
Reactants: Fmoc-Lys(Fmoc)-OH, Fmoc-Gly-OH, Fmoc-Rink-OH, Fmoc-Ramage-OH, TBDMS-HMPB-OH
   Structure of the (Fmoc-Rink)₈-Lys(3)₄-Gly₄-Lys(2)₂-Gly₂-Lys(1)-Gly-AMS Insoluble Support

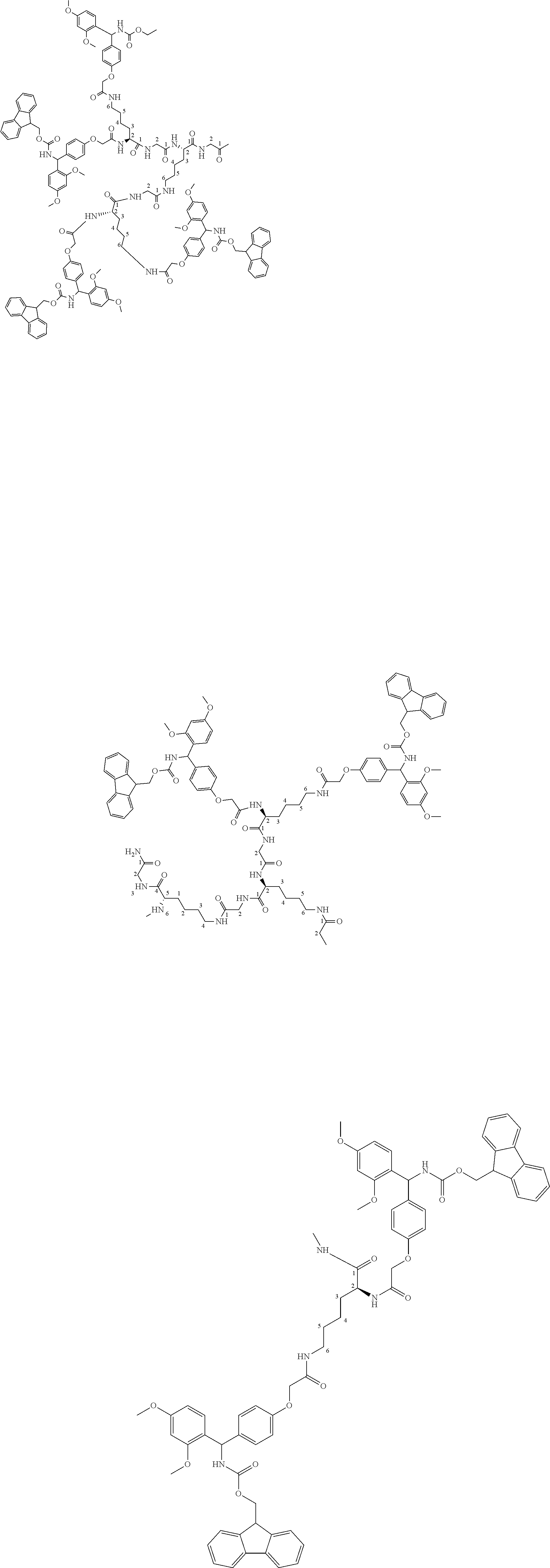

Structure of the (Fmoc-Ramage)₈-K(3)₄-G₄-K(2)₂-G₂-K(1)-G-AMS Insoluble Support

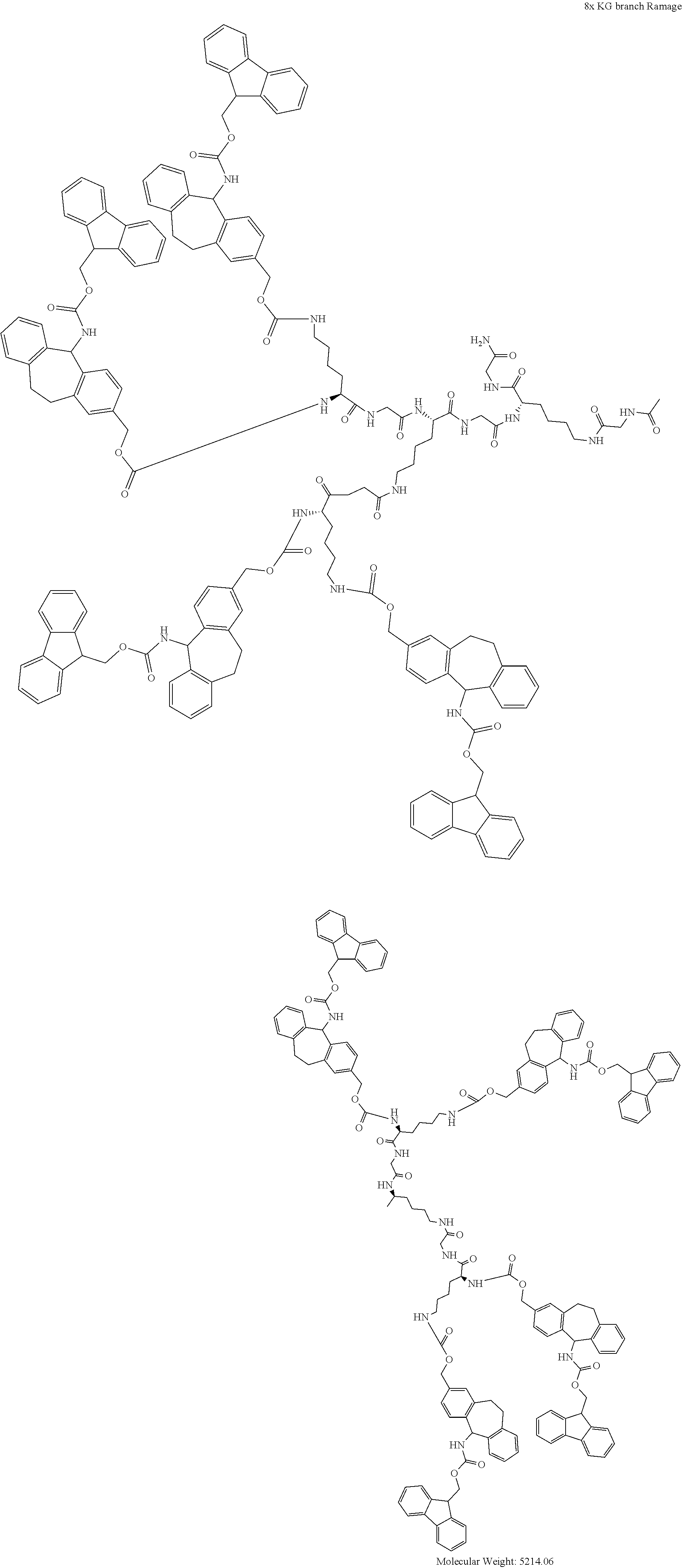

Structure of the (TBDMS-HMPB)₈-K(3)₄-G₄-K(2)₂-G₂-K(1)-G-AMS Insoluble Support

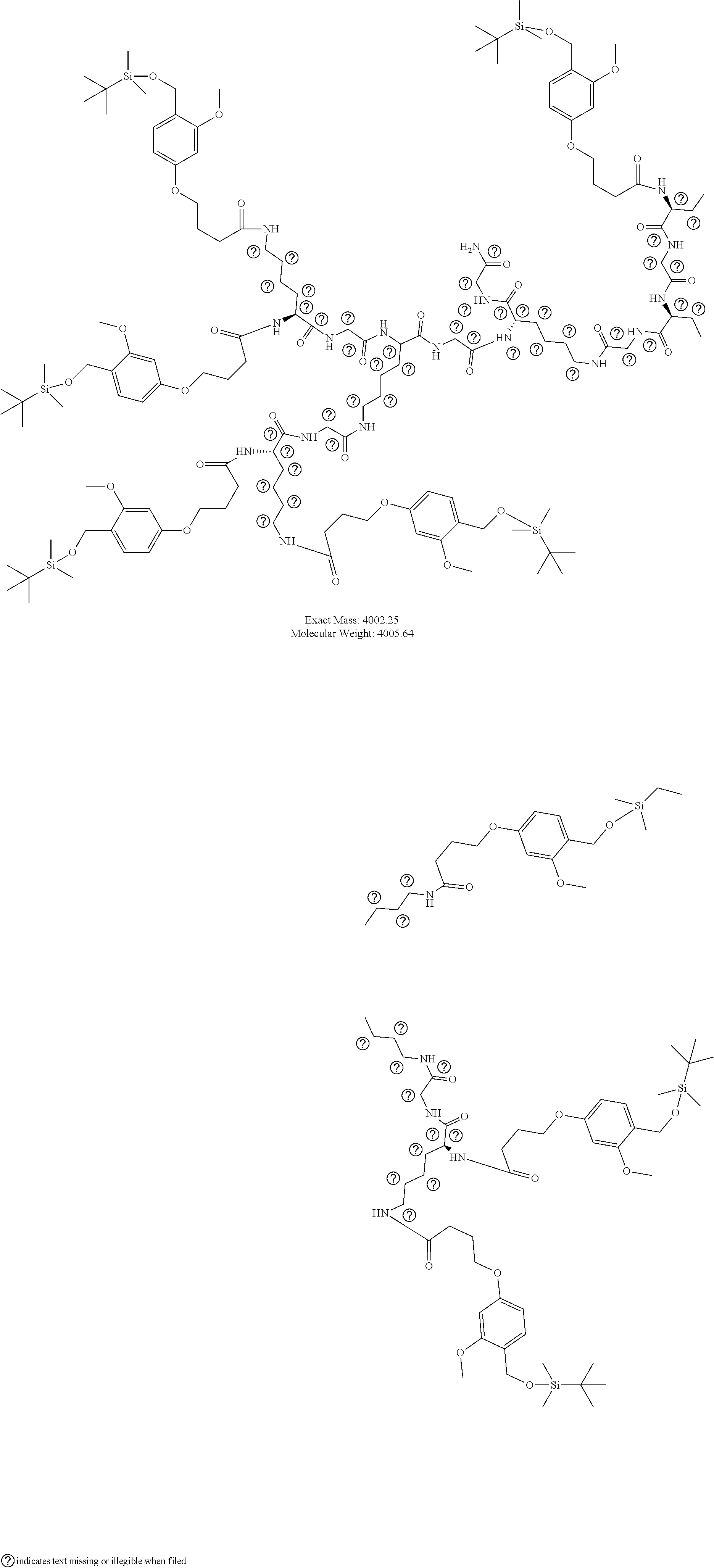

Synthesis scheme for (Linker)₈-Lys(3)₄-Gly₄-Lys(2)₂-Gly₂-Lys(1)-Gly-AMS
Linkers: Fmoc-Rink, Fmoc-Ramage, TBDMS-HMPB

Resin-AMS + Fmoc-Gly-OH
↓ Coupling, deprotection Fmoc
Resin-AMS-Gly-H
↓coupling Fmoc-Lys(Fmoc)-OH
Resin AMS-Gly-Lys(Fmoc)-Fmoc
↓ deprotection Fmoc, coupling Fmoc-Gly-OH
Resin AMS-Gly-Lys(Gly-Fmoc)-Gly-Fmoc
↓ deprotection Fmoc, coupling Fmoc-Lys(Fmoc)
Resin AMS-Gly-Lys(Gly-Lys(Fmoc)-Fmoc)-Gly-
Lys(Fmoc)-Fmoc
↓ deprotection Fmoc, coupling Fmoc-Gly-OH
Resin AMS-Gly-Lys(Gly-Lys(Gly-Fmoc)-Gly-Fmoc)-Gly-
Lys (Gly-Fmoc)-Gly-Fmoc
↓deprotection, coupling Fmoc-Lys(Fmoc)-OH
Resin AMS-Gly-Lys(Gly-Lys(Gly-Lys(Fmoc)-Fmoc)-Gly-
Lys(Fmoc)-Fmoc)-Gly-Lys(Gly-Lys(Fmoc)-Fmoc)-Gly-
Lys(Fmoc)-Fmoc
↓deprotection Fmoc coupling Linker-OH
Resin AMS-Gly-Lys(Gly-Lys(Gly-Lys(Linker)-Linker)-
Gly-Lys(Linker)-Linker)-Gly-Lys(Gly-Lys(Linker)-
Linker)-Gly-Lys(Linker)-Linker
(Resin-AMS-Gly-Lys(1)-Gly₂-Lys(2)₂-Gly₄-Fmoc-Lys(3)₄-
Linker₈

(Fmoc-Rink)₄-(PEG₆)₄-Lys(2)₂-Lys(1)-AMS Insoluble Support [4×(PEG)]

The construct of this modified insoluble support comprises PEG₆ spacers in addition to three lysines and four Fmoc-Rink-linkers. The PEG₆ spacers are positioned between the distal lysines and the Fmoc-Rink-linkers.

The PEG spacer is provided as 2-[2-[2-(Fmoc amino) ethoxy]ethoxy] acetic acid (EAAE). Thus, the PEG₆ spacer is provided by coupling 6 AEEA (PEG): s to each amine group of the secondary (distal) lysines.

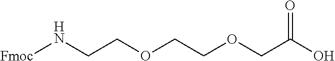

The polymer matrix used for modification is AMS 0.6.

Only lysine has been used where both amine groups are Fmoc protected.
Reactants: Fmoc-Lys(Fmoc)-OH, AEEA, Fmoc-Rink-OH
   Synthesis of Insoluble Support (Fmoc-Rink)₄-(PEG₆)₄-Lys(2)₂-Lys(1)-AMS

Fmoc protected amino acids (K) and AEEA (PEG) are coupled by recurrent reaction steps comprising deprotection of the Fmoc groups followed by the coupling of relevant Fmoc protected amino acids and AEEA. After the consecutive coupling of the last AEEA the linkers are coupled. The amide bonds are formed using equimolar ratio of Fmoc-Lys(Fmoc)-OH, AEEA and Fmoc-Rink-OH, respectively and Oxyma pure (Ethyl cyano (hydroxyimino)acetate: Novabiochem®) and diisopropylcarbodiimide (DIC) at 3-fold excess over the theoretical free amino groups (calculated from the first AMS binding site) in DMF. Fmoc groups are removed prior to each coupling step using two treatments of 25% (v/v) piperidine in DMF (5 and 20 minutes respectively). The amide coupling procedure is allowed to proceed for 2 hours at room temperature RT.

Synthesis scheme for (Fmoc-Rink)₄-(PEG₆)₄-LysK(2)₂-LysK(1)-AMS:

Resin-AMS + Fmoc-Lys(Fmoc)-OH
↓ Coupling, deprotection Fmoc
Resin-AMS-Lys-H
↓coupling Fmoc-Lys(Fmoc)-OH
Resin-AMS-Lys-(Lys(Fmoc)-Fmoc)₂
↓ Deprotection Fmoc, coupling AEEA
Resin-AMS-Resin-AMS-Lys-Lys₂-(PEG6)₄
↓ deprotection Fmoc, coupling Fmoc-Rink-OH
Resin-AMS-Resin-AMS-Lys-Lys₂-(PEG6)₄-(Rink-
Fmoc)₄

Structure of the (Fmoc-Rink)₄-(PEG₆)₄-Lys(2)₂-Lys(1)-NH₂Construct:

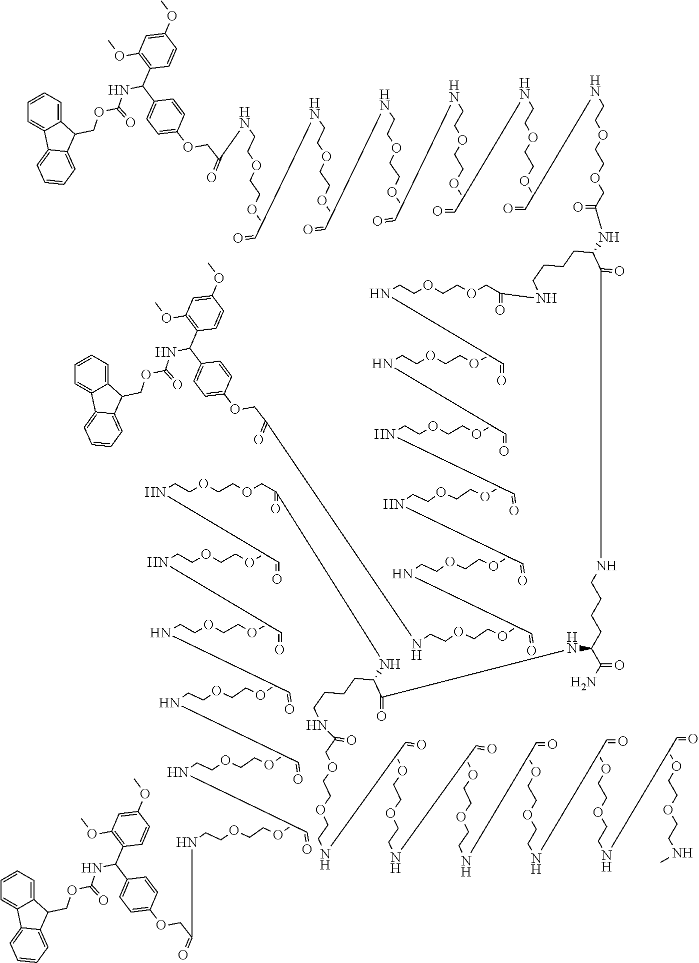

Molecular weight 5971.62

### (Linker)₇-LysGly-Linear-Gly-AMS Insoluble Support [7× Linear]

Linker: Fmoc-Rink, Fmoc-Ramage and HMPB TBDMS
   Molecular Weight of the Respective Constructs without Support:
Fmoc-Rink: Molecular weight (g/mol): 4608.22
Fmoc-Ramage: Molecular weight (g/mol): 4370.12
TBDMS-HMPB: Molecular weight (g/mol): 3312.76

The construct of this insoluble support contains six lysines, three glycines as spacers, and seven linkers selected from Fmoc-Rink-OH, Fmoc-Ramage, HMPB TBDMS, said linkers coupled to four of the six lysines.

In the synthesis of the linear 7× insoluble support two classes of lysine have been used differing in terms of protection groups. One class of lysines have both amine groups protected by Fmoc: Fmoc-Lys(Fmoc) epsilon amine protected by Mtt: Fmoc-Lys(Mtt)-OH.

The construct of the 7× insoluble support is synthesized by first synthesizing a main chain by only coupling Fmoc-Lys(Mtt)-OH. The main chain is the chain where all amide bonds involve an alfa amine. After the synthesis of the main chain comprising three lysines and three glycines, the Mtt is cleaved of with a solution of 30% hexafluoroisopropanol HFIP in DCM for 1 hour at 25° C. In a subsequent step Fmoc-Lys (Fmoc) is coupled to the epsilon amines of each lysine of the main chain. Thereafter, all Fmoc groups are cleaved of using two treatments of 25% (v/v) piperidine in DMF (5 and 20 minutes respectively). Finally, linkers are coupled to the lysines. The seven form 7 secondary (distal) binding sites.

The side chain amines of each lysine of the main chain are only coupled to one lysine, respectively, providing a construct with a configuration which herein is referred to as ‘linear’.

The amide bonds are formed using equimolar ratio of Fmoc-Lys(Fmoc)-OH, Fmoc-Lys(Mtt)-OH, and linker, respectively and Oxyma pure (Ethyl cyano (hydroxyimino)acetate: Novabiochem®) and diisopropylcarbodiimide (DIC) at 3-fold excess over the theoretical free amino groups (calculated from the first AMS binding site) in DMF. Where applicable, Fmoc groups are removed prior to each coupling step using two treatments of 25% (v/v) piperidine in DMF (5 and 20 minutes respectively). The amide coupling procedure is allowed to proceed for 2 hours.

The polymeric matrix used for modification is AMS 0.6.

### Synthesis Scheme for 7× Linear:

Resin-AMS + Fmoc-Gly-OH
↓ Coupling, deprotection Fmoc
Resin-AMS-Gly-H
↓coupling Fmoc-Lys(Mtt)-OH
Resin-AMS-Gly-Lys(Mtt)-Fmoc
↓ deprotection Fmoc, coupling Fmoc-Gly-OH
Resin-AMS-Gly-Lys(Mtt)-Gly-Fmoc
↓ deprotection Fmoc, coupling Fmoc-Lys(Mtt)-OH
Resin-AMS-Gly-Lys(Mtt)-Gly-Lys(Mtt)-Fmoc
↓ deprotection Fmoc, coupling Fmoc-Gly-OH
Resin-AMS-Gly-Lys(Mtt)-Gly-Lys(Mtt)-Gly-Fmoc
↓ deprotection Fmoc, coupling Fmoc-Lys(Mtt)-OH
Resin-AMS-Gly-Lys(Mtt)-Gly-Lys(Mtt)-Gly-Lys(Mtt)-
Fmoc
↓ deprotection Mtt (30% HFIP in DCM)
Resin-AMS-Gly-Lys-Gly-Lys-Gly-Lys-Fmoc
↓ coupling Fmoc-Lys(Fmoc)-OH
Resin-AMS-Gly-Lys(Lys(Fmoc)-Fmoc)-Gly-
Lys(Lys(Fmoc)-Fmoc)-Gly-Lys(Lys(Fmoc)-Fmoc)-Fmoc
↓deprotection Fmoc and coupling Fmoc-Linker-OH
Resin-AMS-Gly-Lys(Lys(Fmoc-Linker)-Fmoc-Linker)-
Gly-Lys(Lys(Fmoc-Linker)-Fmoc-Linker)-Gly-
Lys(Lys(Fmoc-Linker)-Fmoc-Linker)-Fmoc-Linker
Resin-AMS-Gly-Lys(1)(Fmoc-Linker-Lys(2)(Fmoc-
Linker)-Gly-Lys(2)(Fmoc-Linker-Lys(3)(Fmoc-Linker))-
Gly-Lys(3)(Fmoc-Linker)-Lys(4)(Fmoc-Linker)-Fmoc-
Linker =
(Fmoc-Rink)7-LysGly-linear-Gly-AMS)

This modified solid resin has a construct comprising lysines which are only bound to one other lysine, where applicable through a Glycine spacer. The presence of lysines only bound to one other lysine (lysine with other identity) provides an odd number of secondary binding sites: here seven secondary binding sites.

### Structural Formula of the (Fmoc-Rink)₇-LysGly-Linear-Gly-AMS Insoluble Support:

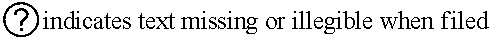

### Structural Formula of the (Fmoc-Ramage)₇-LysGly-Linear-Gly-AMS Insoluble Support:

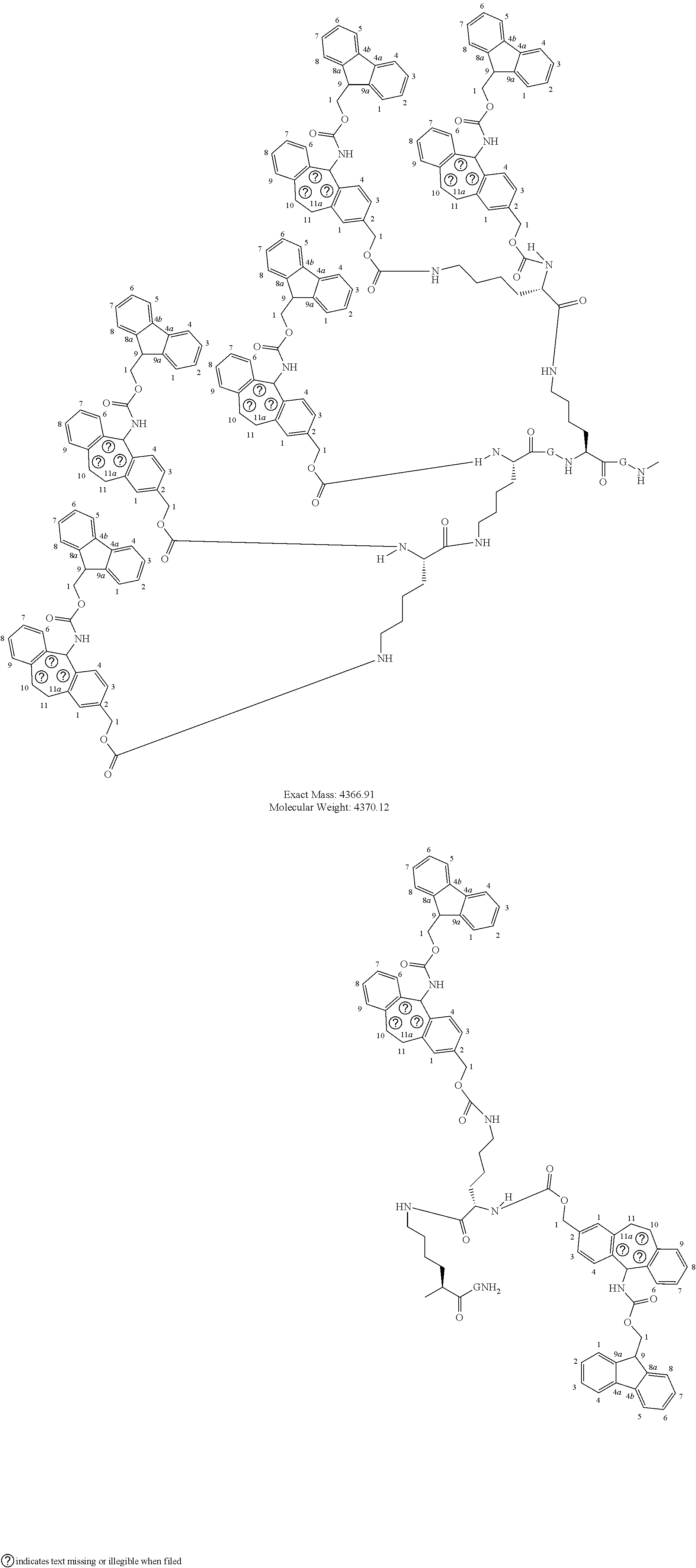

### Structural Formula of the (TBDMS-HMPB)₇-LysGly-Linear-Gly-AMS Insoluble Support:

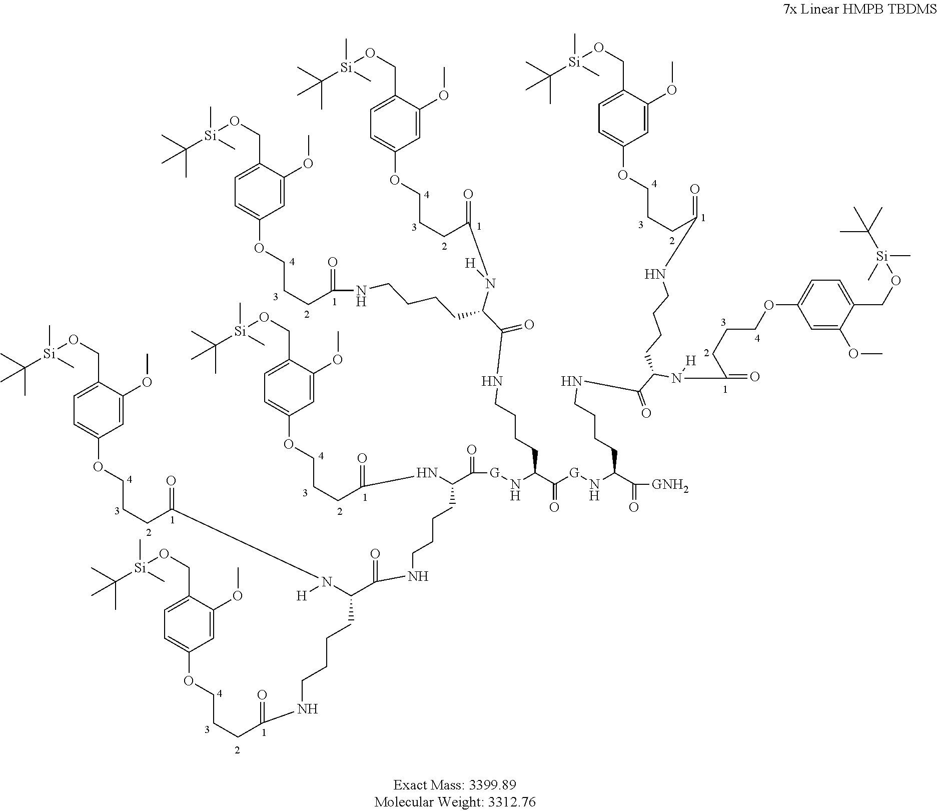

(Fmoc-Rink)₁₆-Lys(4)₈-Gly₈-Lys(3)₄-Gly₄-Lys(2)₂-Gly₂-Lys(1)-Gly-AMS [16×(Gly)]
Molecular weight (g/mol): 1112.49

The 16×(Gly) insoluble support comprises a construct providing a 16-fold increase of the primary binding sites of the base AMS resin. The construct comprises 15 lysines, 15 glycine spacers and 16 Fmoc-Rink-linkers. This construct is symmetrical containing four layers (generations) of lysines, i.e. eight quaternary lysines Lys (4), four tertiary lysines Lys (3), two secondary lysines Lys (2) and a primary lysine Lys(1). Furthermore, all lysines (secondary Lys (2) and tertiary Lys (3)) between the lysine bound to the primary binding site of the base resin and the quaternary lysines Lys (4) are bound to three different lysines. E.g. each of the two secondary lysines Lys (2) are bound to the primary lysine Lys(1) and to two tertiary lysines Lys (3). If all lysines except the distal (distal lysines are in this case the quaternary lysines Lys (4)) and primary are all bound to three lysines then the construct is symmetrical. Also, the number of secondary binding sites are a function of the following equation: Y=2″, where
n denotes the number of layers of lysines in the construct. Here we have 4 layers and, hence, 16 secondary binding sites.

The polymeric matrix used for modification is AMS 0.6.

All lysines are exclusively Fmoc-Lys(Fmoc).
Reactants: Fmoc-Lys(Fmoc), Fmoc-Gly, Fmoc-Rink
   Structural Formula of the (Fmoc-Rink)₁₆-Lys(4)₈-Gly₈-Lys(3)₄-Gly₄-Lys(2)₂-Gly₂-Lys(1)-Gly-AMS Insoluble Support:

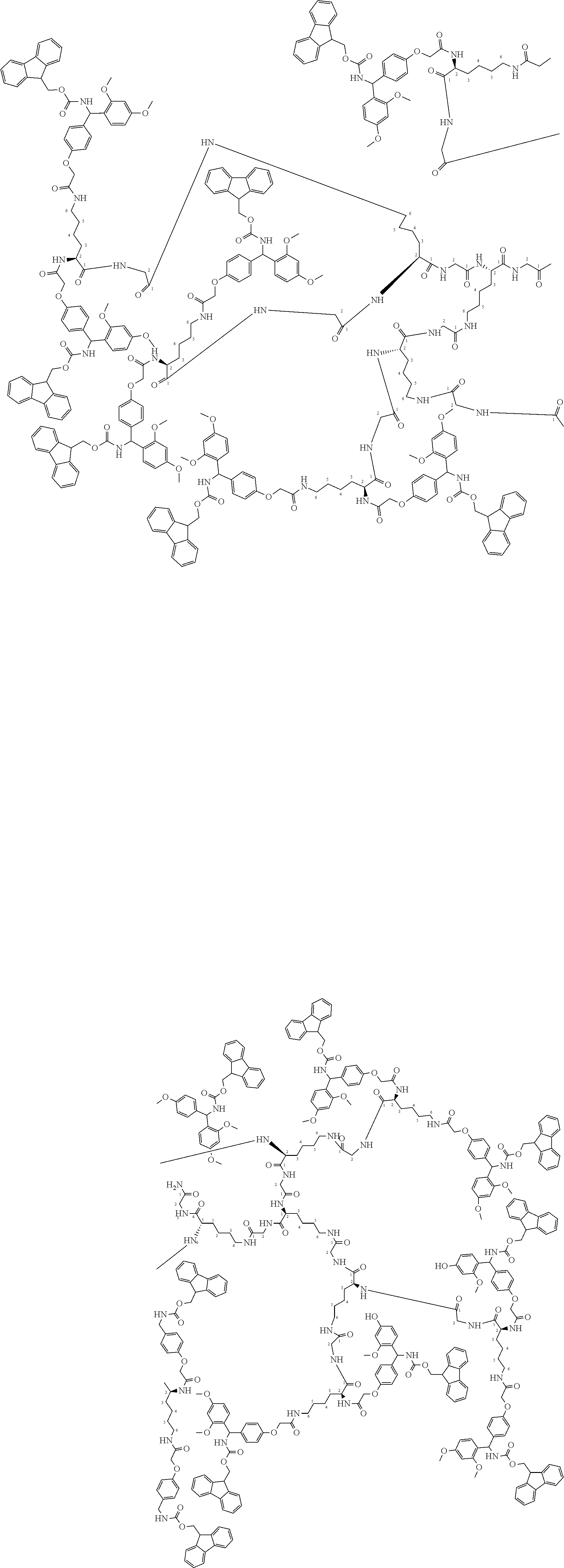

Synthesis of the (Fmoc-Rink)₁₆-Lys(4)₈-Gly₈-Lys(3)₄-Gly₄-Lys(2)₂-Gly₂-Lys(1)-Gly-AMS

The peptide construct is synthesized on a manual peptide synthesis reactor.

Fmoc protected Lysine is coupled to the amines of the AMS. The amide bonds are formed using equimolar ratio of Fmoc-Lys(Fmoc)-OH, Fmoc-Gly-OH, Fmoc-Rink-OH respectively and Oxyma pure (Ethyl cyano (hydroxyimino)acetate: Novabiochem®) and diisopropylcarbodiimide (DIC) at 3-fold excess over the theoretical free amino groups (calculated from the first AMS binding site) in DMF. Fmoc groups are removed prior to each coupling step using two treatments of 25% (v/v) piperidine in DMF (5 and 20 minutes respectively). The amide coupling procedure is allowed to proceed for 2 hours.

### Synthesis Scheme for 16×(Gly)

Resin-AMS + Fmoc-Gly-OH
↓ Coupling, deprotection Fmoc
Resin-AMS-Gly-H
↓coupling Fmoc-Lys(Fmoc)-OH
Resin AMS-Gly-Lys(Fmoc)-Fmoc
↓ deprotection Fmoc, coupling Fmoc-Gly
Resin AMS-Gly-Lys(Gly-Fmoc)-Gly-Fmoc
↓ deprotection Fmoc, coupling Fmoc-Lys(Fmoc)
Resin AMS-Gly-Lys(Gly-Lys(Fmoc)-Fmoc)-Gly-
Lys(Fmoc)-Fmoc
↓ deprotection Fmoc, coupling Fmoc-Gly
Resin AMS-Gly-Lys(Gly-Lys(Gly-Fmoc)-Gly-Fmoc)-Gly-
Lys(Gly-Fmoc)-Gly-Fmoc
↓ deprotection, coupling Fmoc-Lys(Fmoc)-OH
Resin AMS-Gly-Lys(Gly-Lys(Gly-Lys(Fmoc)-Fmoc)-Gly-
Lys(Fmoc)-Fmoc)-Gly-Lys(Gly-Lys(Fmoc)-Fmoc)-Gly-
Lys(Fmoc)-Fmoc
↓deprotection Fmoc, coupling Fmoc-Gly
Resin AMS-Gly-Lys(Gly-Lys(Gly-Lys(Gly-Fmoc)-Gly-
Fmoc)-Gly-Lys(Gly-Fmoc)-Gly-Fmoc)-Gly-Lys(Gly-
Lys(Gly-Fmoc)-Gly-Fmoc)-Gly-Lys(Gly-Fmoc)-Gly-
Fmoc
deprotection Fmoc, coupling Fmoc-Lys(Fmoc)-OH
Resin AMS-Gly-Lys(Gly-Lys(Gly-Lys(Gly-Lys(Fmoc)-
Fmoc)-Gly-Lys(Fmoc)-Fmoc)-Gly-Lys(Gly-Lys(Fmoc)-
Fmoc)-Gly-Lys(Fmoc)-Fmoc)-Gly-Lys(Gly-Lys(Gly-
Lys(Fmoc)-Fmoc)-Gly-Lys(Fmoc)-Fmoc)-Gly-Lys(Gly-
Lys(Fmoc)-Fmoc)-Gly-Lys(Fmoc)-Fmoc
↓ deprotection Fmoc, coupling Fmoc-Rink
Resin AMS-Gly-Lys(Gly-Lys(Gly-Lys(Gly-Lys(Fmoc-
Rink)-Fmoc-Rink)-Gly-Lys(Fmoc-Rink)-Fmoc-Rink)-Gly-
Lys(Gly-Lys(Fmoc-Rink)-Fmoc-Rink)-Gly-Lys(Fmoc-
Rink)-Fmoc-Rink)-Gly-Lys(Gly-Lys(Gly-Lys(Fmoc-
Rink)-Fmoc-Rink)-Gly-Lys(Fmoc-Rink)-Fmoc-Rink)-Gly-
Lys(Gly-Lys(Fmoc-Rink)-Fmoc-Rink)-Gly-Lys(Fmoc-
Rink)-Fmoc-Rink
(Resin-AMS-Gly-Lys(1)-Gly2-Lys(2)₂-Gly₄-Fmoc-
Lys(3)₄-Fmoc-Gly₈-Fmoc-Lys(4)₈-Fmoc-Rink₁₆)

### Synthesis of Polypeptides Using the Insoluble Support of the Invention

Template polypeptide sequences PP1 to PP 6 were synthesized using the following insoluble supports (in detail presented above):

Supports with Rink Linker:
1×: (Fmoc-Rink)-AMS 0.6 (resin without construct)
1×: (Fmoc-Rink)-AMS 0.93 (resin without construct)
1×: (Fmoc-Rink)-AMS 1.95 (resin without construct)
2×(Gly): (Fmoc-Rink)₂-Lys(1)-gly-AMS 0.6
2×(Gly): (Fmoc-Rink)₂-Lys(1)-Gly-AMS 0.93
2×(Gly): (Fmoc-Rink)₂-Lys(1)-Gly-AMS 1.95
4×(Gly): (Fmoc-Rink)₄-Lys(2)₂-Gly₂-Lys(1)-Gly-AMS 0.6
4×(Gly): (Fmoc-Rink)₄-Lys(2)₂-Gly₂-Lys(1)-Gly-AMS 0.93
4×(Gly): (Fmoc-Rink)₄-Lys(2)₂-Gly₂-Lys(1)-Gly-AMS 1.95
8×(Gly): (Fmoc-Rink)₈-Lys(3)₄-Gly₄-Lys(2)₂-Gly₂-Lys(1)-Gly-AMS 0.6
   Supports with Ramage Linker:
1×: (Fmoc-Ramage)-AMS 0.6 (resin without construct)
1×: (Fmoc-Ramage)-AMS 0.93 (resin without construct)
1×: (Fmoc-Ramage)-AMS 1.95 (resin without construct)
2×(Gly): (Fmoc-Ramage)₂-Lys(1)-Gly-AMS 0.6
2×(Gly): (Fmoc-Ramage)₂-Lys(1)-Gly-AMS 0.93
2×(Gly): (Fmoc-Ramage)₂-Lys(1)-Gly-AMS 1.95
4×(Gly): (Fmoc-Ramage)₄-Lys(2)₂-Gly₂-Lys(1)-Gly-AMS 0.6
4×(Gly): (Fmoc-Ramage)₄-Lys(2)₂-Gly₂-Lys(1)-Gly-AMS 0.93
4×(Gly): (Fmoc-Ramage)₄-Lys(2)₂-Gly₂-Lys(1)-Gly-AMS 1.95
   Supports with HMPB TBDMS Linker:
1×: HMPB TBDMS AMS 0.6 (resin without construct)
1×: HMPB TBDMS AMS 0.93 (resin without construct)
4×(Gly): (HMPB TBDMS)₄-Lys(2)₂-Gly₂-Lys(1)-Gly-AMS 0.6
8×(Gly): (HMPB TBDMS)₈-Lys(3)₄-Gly₄-Lys(2)₂-Gly₂-Lys(1)-Gly-AMS 0.6

The reference resin is an amino methyl (AM/AMS) polystyrene resin cross-linked with 1% divinylbenzene in the 100-200 mesh size range (75-150 μm) with a degree of substitution of 0.6, 0.93 and 1.95 mmol/gram respectively.

The following polypeptides were synthesized:

PP 1: WLFAGGPSSGAPPPS (15mer)
PP 2: YAEGTFTSDYSIALDKIAQKAFVQWLIAGGPSSGAPPPS
(39mer)
PP 3: HGEGTFTSDLSKQMEEEAVRLFIEWLKNGGPSSGAPPPS
(39mer)
PP 4: fFPRPGGGGNGDFEEIPEEYL (20mer)
PP 5: FVQYLIQG (8mer)
PP 6: HAEGTFTSDVSSYLEGQAAKEFIAWLVRGRG (31mer)

### Constructs:

1×: No construct only unmodified resin with linker as indicated below.
2×(Gly): -Gly-Lys(1)-Linker₂
4×(Gly): -Gly-Lys(1)-Gly₂-Lys(2)₂-Linker₄
8×(Gly): -Gly-Lys(1)-Gly₂-Lys(2)₂-Gly₄-Lys(3)₄-Linkers

Tables 8 to 14 present various data related to the polypeptide synthesis.

In some columns the assembly yield [%] and cleavage yield [%] are indicated. These parameters are calculated as follows:

$$Assembly yield = \frac{m ( peptide - resin ) - m ( resin )}{MW ( protected peptide \times scale} \times 100 \qquad ( 1 )$$

$$Cleavage yield = \frac{m ( crude peptide )}{MW ( peptide salt ) \times scale} \times 100 \qquad ( 2 )$$

Synthesis Protocol for PP 1 with DMF as Solvent

Starting resins (400 mg each) were introduced into 25 mL syringes and swollen in DMF (6.5 mL/g, 2×1 h) for initial swelling measurements prior to their transfer to Symphony X reactors. Fmoc groups were deprotected prior to each coupling step using 20% piperidine in DMF (1×10 min+1×20 min) at room temperature. All amino acid couplings were carried out using an equimolar ratio (1:1:1) of Fmoc amino acid (0.30M in DMF), Oxyma (0.90M in DMF) and N′N′-Diisopropylcarbodiimide (0.90M in DMF) at 5-fold molar excess compared to synthesis scale. Couplings were carried out without pre-activation for 1 h 30 min at room temperature.

After syntheses completion, resins were transferred again to the 25 mL syringes to measure their final swellings then washed with isopropanol (iPrOH, 3×) and methyl tert-butyl ether (MTBE, 3×) prior to their drying under vacuum overnight.

Dried Fmoc-protected peptidyl resins were swollen again in DMF (6.5 mL/g, 2×1 h). Fmoc groups were deprotected using 20% piperidine in DMF (1×10 min+1×20 min) at room temperature. Next, resins were washed with DMF (7×), iPrOH (3×) and MTBE (3×) prior to their re-drying under vacuum overnight.

Dried Fmoc-deprotected peptides were cleaved from resins using a cleavage cocktail containing TFA (Trifluoroacetic acid)/H₂O/DTT (Dithiothreitol) (13 mL/g of peptidyl resin, 85/5/5 v/v/w) for 2 h at room temperature. TIS (5% v) was then added and stirring was continued for another 1 h. The resin was filtered out and washed with TFA (3 mL/g of peptidyl resin). Peptides were precipitated with MTBE (10-folds the volume of TFA) at 0° C. The resulting suspensions were transferred to 50 ml conical centrifuge tubes and centrifuged at 2500 rcf for 10 min prior to supernatants' decantation. The crude solid products were washed again with cold MTBE (5×) and dried under vacuum overnight.
Molecular weight: 1426.60
Calculated mass: +2/2=714.30
Observed mass (AMS 0.93 2×(Gly)) +2/2=714.05
Observed mass (AMS 1.95 2×(Gly)): +2/2=714.43
Observed mass (AMS 1.95 4×(Gly)): +2/2=714.64

All 15 amino acids used were Fmoc-protected.

Side chains protecting groups involved: tBu, Boc: S=Ser(tBu) and W=Trp(Boc)
Molecular weight of protected PP 1:1917.28 g/mol
Molecular weight of unprotected PP 1:1426.60 g/mol

TABLE 8
Data synthesis of 15mer polypeptide: WLFAGGPSSGAPPPS: PP 1 Solvent for the synthesis:
DMF Mass of matrix at the beginning of synthesis after coupling of Rink linker: 400 mg

| | | | | Theoretical | Final mass | Final volume |
|---|---|---|---|---|---|---|
| | | Substitution | | mass of Fmoc | of Fmoc | of Fmoc |
| Unmodified | | at beginning | Synthesis | protected | protected | protected |
| matrix/resin | Type of | of synthesis | scale | peptidyl resin | peptidyl resin | peptidyl resin |
| [mmol/g] | construct | [mmol/g] | [mmol] | [mg] | [mg] | [mL] |
| AMS 0.6 | 1X | 0.48 | 0.192 | 725 | 660 | 4.6 |
| AMS 0.6 | 2X(Gly) | 0.68 | 0.272 | 861 | 800 | 4.3 |
| AMS 0.6 | 4X(Gly) | 0.90 | 0.360 | 1010 | 930 | 4.0 |
| AMS 0.6 | 8X(Gly) | 0.98 | 0.392 | 1065 | 987 | 3.7 |
| AMS 0.93 | 1X | 0.62 | 0.248 | 820 | 760 | 4.3 |
| AMS 0.93 | 2X(Gly) | 0.82 | 0.328 | 956 | 788 | 3.6 |
| AMS 0.93 | 4X(Gly) | 1.10 | 0.440 | 1146 | 680 | 3.3 |
| AMS 1.95 | 1X | 1.08 | 0.432 | 1132 | 990 | 3.8 |
| AMS 1.95 | 2x(Gly) | 1.14 | 0.456 | 1173 | 995 | 3.3 |
| AMS 1.95 | 4X(Gly) | 1.20 | 0.480 | 1214 | 684 | 2.5 |

| | | | | | | SPPS reactor |
|---|---|---|---|---|---|---|
| | | | | Theoretical | | throughput |
| HPLC purity | | | | crude peptide | | (mass after |
| of crude | Weight | Assembly | Cleavage | mass after | Crude peptide | cleavage/final |
| peptide | gain | yield | yield | cleavage | after cleavage | volume) |
| [%] | [mg] | [%] (1) | [%] (2) | [mg] | [mg] | [g/L] |
| 82.4 | 303 | 82 | 71 | 296 | 211 | 45.9 |
| 89.3 | 460 | 88 | 88 | 419 | 370 | 86.1 |
| 81.3 | 610 | 89 | 88 | 555 | 486 | 114.0 |
| 78.2 | 674 | 90 | 87 | 604 | 523 | 141.4 |
| 74.4 | 415 | 87 | 66 | 382 | 250 | 58.1 |
| 85.5 | 461 | 73 | 66 | 505 | 330 | 91.7 |
| 84.5 | 378 | 45 | 64 | 678 | 430 | 130.3 |
| 81.8 | 686 | 83 | 69 | 666 | 460 | 121.1 |
| 83.7 | 696 | 80 | 70 | 703 | 490 | 148.5 |
| 83.2 | 391 | 43 | 58 | 740 | 429 | 171.6 |

Implementation of support of invention significantly increases throughput while essentially maintaining purity or even increasing purity.

Synthesis Protocol for PP 1 with DMSO/EtOAc (3:7 v/v) and DMF as Solvent

Starting resins (400 mg each) were introduced into 25 mL syringes and swollen in DMF (6.5 mL/g, 2×1 h). Syntheses were carried out manually where syringes were shaken with an Activo-PLS 4×4 synthesizer purchased from Activotec at 400 rpm in horizontal position. Fmoc groups were deprotected prior to each coupling step using 20% piperidine in DMSO/EtOAc (3:7, v/v) or DMF (1×10 min+1×20 min) at room temperature. Following Fmoc-deprotection, resins were washed using DMSO/EtOAc (3:7, v/v) or DMF (7×6.5 mL/g of starting resin). All amino acid couplings were carried out using an equimolar ratio (1:1:1) of Fmoc amino acid in DMSO/EtOAc (3:7, v/v) or DMF (0.30M), Oxyma and N′N′-Diisopropylcarbodiimide at 2-fold molar excess compared to synthesis scale. Fmoc amino acids were dissolved in the corresponding solvent/solvent mixture (0.30M) followed by the addition of Oxyma and N′N′-Diisopropylcarbodiimide and stirred for 5 min at room temperature before being introduced into the resin-containing syringes. Couplings were carried out for 1 h 30 min at room temperature. Following coupling, resins were washed using DMSO/EtOAc (3:7, v/v) or DMF (2×6.5 mL/g of starting resin).

After syntheses completion, final resin swellings were measured then resins were washed with (iPrOH, 3×) and methyl tert-butyl ether (MTBE, 3×) prior to their drying under vacuum overnight.

Dried Fmoc-protected peptidyl resins were swollen again in DMF (6.5 mL/g, 2×1 h). Fmoc groups were deprotected using 20% piperidine in DMF (1×10 min+1×20 min) at room temperature. Next, resins were washed with DMF (7×), iPrOH (3×) and MTBE (3×) prior to their re-drying under vacuum overnight.

Dried Fmoc-deprotected peptides were cleaved from resins using a cleavage cocktail containing TFA/H₂O/DTT (13 mL/g of peptidyl resin, 85/5/5 v/v/w) for 2 h at room temperature. TIS (5% v) was then added and stirring was continued for another 1 h. The resin was filtered out and washed with TFA (3 mL/g of peptidyl resin). Peptides were precipitated with MTBE (10-folds the volume of TFA) at 0° C. The resulting suspensions were transferred to 50 ml conical centrifuge tubes and centrifuged at 2500 rcf for 10 min prior to supernatants' decantation. The crude solid products were washed again with cold MTBE (5×) and dried under vacuum overnight.

Mass of resin at beginning of synthesis after coupling of Rink Amide linker: 400 mg

Side chains protecting groups involved: tBu, Boc: S=Ser(tBu) and W=Trp(Boc)
Molecular weight of protected PP 1:1917.28 g/mol
Molecular weight of unprotected PP 1:1426.60 g/mol

TABLE 9
Data Synthesis of 15mer polypeptide: WLFAGGPSSGAPPPS: PP 1 Solvent for the synthesis: DMSO/EtOAc (3:7) except
first row AMS 0.6 with DMF Mass of matrix at the beginning of synthesis after coupling of Rink linker: 400 mg

| | | | | Theoretical | Final mass | Final volume | Theoretical | |
|---|---|---|---|---|---|---|---|---|
| | | Substitution | | mass of Fmoc | of Fmoc | of Fmoc | crude peptide | |
| Unmodified | | at beginning | Synthesis | protected | protected | protected | mass after | Assembly |
| matrix/resin | Type of | of synthesis | scale | peptidyl resin | peptidyl resin | peptidyl resin | cleavage | yield |
| [mmol/g] | construct | [mmol/g] | [mmol] | [mg] | [mg] | [mL] | [mg] | [%] (1) |
| AMS 0.6 | 1X | 0.48 | 0.192 | 725 | 650 | 4.3 | 296 | 77 |
| DMF | | | | | | | | |
| AMS 0.6 | 1X | 0.48 | 0.192 | 725 | 640 | 2.7 | 296 | 80 |
| AMS 0.6 | 2X(Gly) | 0.68 | 0.272 | 861 | 808 | 2.8 | 419 | 90 |
| AMS 0.6 | 4X(Gly) | 0.90 | 0.360 | 1010 | 950 | 2.8 | 555 | 91 |
| AMS 0.6 | 8X(Gly) | 0.98 | 0.392 | 1065 | 1006 | 2.7 | 604 | 92 |

| | | | | | SPPS reactor |
|---|---|---|---|---|---|
| | | | | | throughput |
| HPLC purity | | | | | (mass after |
| of crude | | | Cleavage | Crude peptide | cleavage/final |
| peptide | Weight gain | | yield | after cleavage | volume) |
| [%] | [mg] | Solvent | [%] (2) | [mg] | [g/L] |
| 83.8 | 283 | DMF | 87 | 264 | 61.4 |
| 85.1 | 293 | DMSO/EtOAc | 264 | 258 | 95.6 |
| 81.5 | 468 | DMSO/EtOAc | 93 | 388 | 138.6 |
| 81.9 | 630 | DMSO/EtOAc | 96 | 530 | 189.3 |
| 80.0 | 693 | DMSO/EtOAc | 99 | 595 | 220.4 |

Synthesis Protocol for PP 2 (YAEGTFTSDYSIALDKIAQKAFVQWLIAGGPSSGAPPPS) with DMF as Solvent

Starting resins (200 mg each) were introduced into 12 mL syringes and swollen in DMF (6.5 mL/g, 2×1 h) for initial swelling measurements prior to their transfer to Symphony X reactors. Fmoc groups were deprotected prior to each coupling step using 20% piperidine in DMF (1×10 min+1×20 min) at room temperature. All amino acid couplings were carried out using an equimolar ratio (1:1:1) of Fmoc amino acid (0.30M in DMF), Oxyma (0.90M in DMF) and N′N′-Diisopropylcarbodiimide (0.90M in DMF) at 5-fold molar excess compared to synthesis scale. Couplings were carried out without pre-activation for 2 h at room temperature except for Thr5 which was coupled during 8 h and Ile^{12 and 17} which were coupled during 6 h.

After syntheses completion, resins were transferred again to the 12 mL syringes to measure their final swellings then washed with (iPrOH, 3×) and methyl tert-butyl ether (MTBE, 3×) prior to their drying under vacuum overnight.

Dried Boc-protected peptides were cleaved from resins using a cleavage cocktail containing TFA/H₂O/DTT/TIS (13 mL/g of peptidyl resin, 85/5/5/5 v/v/w/v) for 3 h at room temperature. The resin was filtered out and washed with TFA (3 mL/g of peptidyl resin). Peptides were precipitated with MTBE (10-folds the volume of TFA) at 0° C. The resulting suspensions were transferred to 50 ml conical centrifuge tubes and centrifuged at 2500 rcf for 10 min prior to supernatants' decantation. The crude solid products were washed again with cold MTBE (5×) and dried under vacuum overnight.
Molecular weight: 4041.54
Calculated mass: +3/3=1348.26; +4/4=1011.38
Observed mass (AMS 0.6 2×(Gly)): +3/3=1347.98; +4/4=1011.70
Observed mass (AMS 0.93 4×(Gly)): +3/3=1348.6; +4/4=1011.67

TABLE 10
Data Synthesis of 39mer polypeptide: YAEGTFTSDYSIALDKIAQKAFVQWLIAGGPSSGAPPPS:
PP 2 Solvent for the synthesis: DMF Mass of matrix at the beginning
of synthesis after coupling of Ramage linker: 200 mg

| | | | | Theoretical | Final mass | Final volume |
|---|---|---|---|---|---|---|
| | | Substitution | | mass of Boc | of Boc | of Boc |
| Unmodified | | at beginning | Synthesis | protected | protected | protected |
| matrix/resin | Type of | of synthesis | scale | peptidyl resin | peptidyl resin | peptidyl resin |
| [mmol/g] | construct | [mmol/g] | [mmol] | [mg] | [mg] | [mL] |
| AMS 0.6 | 1X | 0.39 | 0.078 | 619 | 560 | 1.9 |
| AMS 0.6 | 2X(Gly) | 0.62 | 0.124 | 866 | 840 | 2.3 |
| AMS 0.6 | 4X(Gly) | 1.28 | 0.224 | 1404 | 1060 | 2.4 |
| AMS 0.6 | 8X(Gly) | 1.16 | 0.232 | 1447 | 870 | 1.9 |
| AMS 0.93 | 1X | 0.49 | 0.098 | 727 | 692 | 2.5 |
| AMS 0.93 | 2X(Gly) | 1.00 | 0.200 | 1275 | 880 | 2.2 |
| AMS 0.93 | 4X(Gly) | 1.04 | 0.208 | 1318 | 1002 | 2.1 |
| AMS 1.95 | 1X | 0.82 | 0.164 | 1081 | 1030 | 2.7 |
| AMS 1.95 | 2x(Gly) | 0.94 | 0.188 | 1210 | 823 | 2.2 |
| AMS 1.95 | 4X(Gly) | 0.90 | 0.180 | 1167 | 421 | 1.0 |

| | | | SPPS reactor |
|---|---|---|---|
| Theoretical | | | throughput |
| crude peptide | | | (mass after |
| mass after | Crude peptide | HPLC purity | cleavage/final |
| cleavage | after cleavage | after cleavage | volume) |
| [mg] | [mg] | [%] | [g/L] |
| 333 | 280 | 54.3 | 147.4 |
| 533 | 500 | 57.7 | 217.4 |
| 962 | 680 | 56.3 | 283.3 |
| 991 | 550 | 23.4 | 289.5 |
| 418 | 380 | 54.5 | 152.0 |
| 854 | 661 | 54.8 | 300.5 |
| 888 | 640 | 39.1 | 304.8 |
| 700 | 650 | 49.6 | 240.8 |
| 803 | 674 | 24.1 | 306.4 |
| 769 | 295 | 10.6 | 295.0 |

| Weight gain | Assembly yield | Cleavage yield |
|---|---|---|
| [mg] | [%] (1) | [%] (2) |
| 377 | 86 | 84 |
| 668 | 96 | 94 |
| 910 | 73 | 71 |
| 722 | 56 | 56 |
| 514 | 94 | 91 |
| 725 | 65 | 75 |
| 848 | 73 | 72 |
| 867 | 95 | 93 |
| 665 | 63 | 84 |
| 261 | 26 | 38 |

Synthesis Protocol for PP 3 (HGEGTFTSDLSKQMEEEAVRLFXEWLKNGGPSSGAPPPS) with DMF as Solvent

Starting resins (200 mg each) were introduced into 12 mL syringes and swollen in DMF (6.5 mL/g, 2×1 h) for initial swelling measurements prior to their transfer to Symphony X reactors. Fmoc groups were deprotected prior to each coupling step using 20% piperidine in DMF (1×10 min+1×20 min) at room temperature. All amino acid couplings were carried out using an equimolar ratio (1:1:1) of Fmoc amino acid (0.30M in DMF), Oxyma (0.90M in DMF) and N′N′-Diisopropylcarbodiimide (0.90M in DMF) at 5-fold molar excess compared to synthesis scale. Couplings were carried out without pre-activation for 2 h at room temperature except for Thr5 which was coupled during 8 h and Ile^{12 and 17} which were coupled during 6 h.

After syntheses completion, resins were transferred again to the 12 mL syringes to measure their final swellings then washed with (iPrOH, 3×) and methyl tert-butyl ether (MTBE, 3×) prior to their drying under vacuum overnight.

Dried Boc-protected peptides were cleaved from resins using a cleavage cocktail containing TFA/H₂O/DTT/TIS (13 mL/g of peptidyl resin, 85/5/5/5 v/v/w/v) for 3 h at room temperature. The resin was filtered out and washed with TFA (3 mL/g of peptidyl resin). Peptides were precipitated with MTBE (10-folds the volume of TFA) at 0° C. The resulting suspensions were transferred to 50 ml conical centrifuge tubes and centrifuged at 2500 rcf for 10 min prior to supernatants' decantation. The crude solid products were washed again with cold MTBE (5×) and dried under vacuum overnight.
Molecular weight: 4186.63
Calculated mass: +3/3=1396.54; +4/4=1047.65
Observed mass (AMS 0.6 2×(Gly)): +3/3=1397.12; +4/4=1048.11
Observed mass (AMS 0.6 4×(Gly)): +3/3=1396.89; +4/4=1047.65

All amino acids used were Fmoc-protected except for the last amino acid (Tyr) which was Boc-protected.
Side chains protecting groups involved: tBu, Boc, Trt: S=Ser(tBu), W=Trp(Boc), Q=Gln(Trt), K=Lys(Boc), D=Asp(OtBu), Y=Tyr(tBu), T=Thr(tBu), E=Glu(OtBu)
Molecular weight of protected 39-mer: 5596.22 g/mol
Molecular weight of unprotected 39-mer: 4041.54 g/mol

TABLE 11
Data Synthesis of 39mer polypeptide: HGEGTFTSDLSKQMEEEAVRLFXEWLKNGGPSSGAPPPS:
PP 3 Solvent for the synthesis: DMF Mass of matrix at the beginning
of synthesis after coupling of Ramage linker: 200 mg

| | | | | Theoretical | Final mass | Final volume |
|---|---|---|---|---|---|---|
| | | Substitution | | mass of Boc | of Boc | of Boc |
| Unmodified | | at beginning | Synthesis | protected | protected | protected |
| matrix/resin | Type of | of synthesis | scale | peptidyl resin | peptidyl resin | peptidyl resin |
| [mmol/g] | construct | [mmol/g] | [mmol] | [mg] | [mg] | [mL] |
| AMS 0.6 | 1X | 0.39 | 0.078 | 674 | 360 | 2.4 |
| AMS 0.6 | 2X(Gly) | 0.62 | 0.124 | 953 | 690 | 2.9 |
| AMS 0.6 | 4X(Gly) | 1.28 | 0.224 | 1560 | 993 | 3.1 |

| | | | SPPS reactor |
|---|---|---|---|
| Theoretical | | | throughput |
| crude peptide | | | (mass after |
| mass after | Crude peptide | HPLC purity | cleavage/final |
| cleavage | after cleavage | after cleavage | volume) |
| [mg] | [mg] | [%] | [g/L] |
| 362 | 339 | 53.3 | 141.3 |
| 576 | 603 | 52.4 | 207.9 |
| 1040 | 885 | 43.5 | 275.8 |

| Weight gain | Assembly yield | Cleavage yield |
|---|---|---|
| [mg] | [%] (1) | [%] (2) |
| 167 | 34 | 94 |
| 518 | 66 | 99 |
| 843 | 60 | 82 |

The implementation of a support of the invention significantly increases throughput at good/commercially relevant/useful purity.

Synthesis Protocol for PP 4 (fPRPGGGGNGDFEEIPEEYL) with DMF as Solvent

Starting resins (200 mg each) were introduced into 12 mL syringes and swollen in DMF (6.5 mL/g, 2×1 h) for initial swelling measurements prior to their transfer to Symphony X reactors. Fmoc groups were deprotected prior to each coupling step using 20% piperidine in DMF (1×10 min+1×20 min) at room temperature. All amino acid couplings were carried out using an equimolar ratio (1:1:1) of Fmoc amino acid (0.30M in DMF), Oxyma (0.90M in DMF) and N′N′-Diisopropylcarbodiimide (0.90M in DMF) at 5-fold molar excess compared to synthesis scale. Couplings were carried out without pre-activation for 1 h 30 min at room temperature.

After syntheses completion, resins were transferred again to the 12 mL syringes to measure their final swellings then washed with (iPrOH, 3×) and methyl tert-butyl ether (MTBE, 3×) prior to their drying under vacuum overnight.

Dried Boc-protected peptides were cleaved from resins using a cleavage cocktail containing TFA/H₂O/DTT/TIS (13 mL/g of peptidyl resin, 85/5/5/5 v/v/w/v) for 3 h at room temperature. The resin was filtered out and washed with TFA (3 mL/g of peptidyl resin). Peptides were precipitated with MTBE (10-folds the volume of TFA) at 0° C. The resulting suspensions were transferred to 50 ml conical centrifuge tubes and centrifuged at 2500 rcf for 10 min prior to supernatants' decantation. The crude solid products were washed again with cold MTBE (5×) and dried under vacuum overnight.
Molecular weight: 2180.32
Calculated mass: +2/2=1091.16
Observed mass (AMS 0.6 4×(Gly)): +2/2=1091.02
Observed mass (AMS 0.6 8×(Gly)): +2/2=1091.06

All amino acids used were Fmoc-protected except for the last amino acid (D-Phe) which was Boc-protected.
Side chains protecting groups involved: tBu, Trt, Pbf: Y=Tyr(tBu), E=Glu(OtBu), D=Asp(OtBu), N=Asn(Trt), Arg(Pbf)
Molecular weight of protected 20-mer: 3233, 86 g/mol
Molecular weight of unprotected 20-mer: 2180.32 g/mol

TABLE 12
Data Synthesis of 20mer polypeptide: fPRPGGGGNGDFEEIPEEYL: PP
4 Solvent for the synthesis: DMF Mass of matrix at the beginning
of synthesis after coupling of HMPB-leucine linker: 200 mg

| | | | | Theoretical | Final mass | Final volume |
|---|---|---|---|---|---|---|
| | | Substitution | | mass of Boc | of Boc | of Boc |
| Unmodified | | at beginning | Synthesis | protected | protected | protected |
| matrix/resin | Type of | of synthesis | scale | peptidyl resin | peptidyl resin | peptidyl resin |
| [mmol/g] | construct | [mmol/g] | [mmol] | [mg] | [mg] | [mL] |
| AMS 0.93 | 1X | 0.49 | 0.098 | 484 | 166 | 2.4 |
| AMS 0.6 | 4X(Gly) | 0.88 | 0.176 | 710 | 451 | 2.5 |
| AMS 0.6 | 8X(Gly) | 0.90 | 0.180 | 722 | 490 | 1.7 |

| | | | SPPS reactor |
|---|---|---|---|
| Theoretical | | | throughput |
| crude peptide | | | (mass after |
| mass after | Crude peptide | HPLC purity | cleavage/final |
| cleavage | after cleavage | after cleavage | volume) |
| [mg] | [mg] | [%] | [g/L] |
| 236 | 135 | 78.8 | 56.3 |
| 424 | 320 | 76.5 | 128.0 |
| 433 | 290 | 46.1 | 170.6 |

| Weight gain | Assembly yield | Cleavage yield |
|---|---|---|
| [mg] | [%] (1) | [%] (2) |
| 62 | 20 | 57 |
| 290 | 53 | 76 |
| 330 | 59 | 67 |

Application of support of invention significantly increases throughput while essentially preserving purity.

Synthesis Protocol for PP 5 (FVQYLIQG) with DMF as Solvent

Starting resins (100 mg each Fmoc-HMPB-Gly-AMS 0.60) were introduced into 12 mL syringes and swollen in DMF (6.5 mL/g, 2×1 h) for initial swelling measurements prior to their transfer to Symphony X reactors. Fmoc groups were deprotected before prior to each coupling step using 20% piperidine in DMF (1×10 min+1×20 min) at room temperature. All amino acid couplings were carried out using an equiamolar ratio (1:1:1) of Fmoc amino acid (0.30M in DMF), Oxyma (0.90M in DMF) and N′N′-Diisopropylcarbodiimide (0.90M in DMF) at 5-fold molar excess compared to synthesis scale. Couplings were carried out without pre-activation for 1 h 30 min at room temperature.

After syntheses completion, resins were transferred again to the 12 mL syringes to measure their final swellings then washed with (iPrOH, 3×) and methyl tert-butyl ether (MTBE, 3×) prior to their drying under vacuum overnight.

Dried Fmoc-protected peptide fragments were cleaved from resins using a soft cleavage cocktail containing 3% TFA in dichloromethane (DCM) for 15 min. This treatment was done 3 times and each time the resin was filtered out and resulting solution was collected. Combined collected solutions were evaporated under vacuum and the obtained concentrated oil was precipitated in MTBE/Heptane (1:1, v/v) prior to filtration and drying under vacuum overnight.

All amino acids used were Fmoc-protected.
Side chains protecting groups involved: tBu, Trt: Q=Gln(Trt), Y=Tyr(tBu)
Molecular weight of protected 8-mer: 1730, 1 g/mol

TABLE 13
Synthesis of 8mer polypeptide: FVQYLIQG: PP 5 Solvent for the synthesis: DMF Mass of
matrix at the beginning of synthesis after coupling of HMPB-Glycine linker: 100 mg

| | | | | Theoretical | Final mass | Final volume |
|---|---|---|---|---|---|---|
| | | Substitution | | mass of Fmoc- | of Fmoc | of Fmoc |
| Unmodified | | at beginning | Synthesis | protected | protected | protected |
| matrix/resin | Type of | of synthesis | scale | peptidyl resin | peptidyl resin | peptidyl resin |
| [mmol/g] | construct | [mmol/g] | [mmol] | [mg] | [mg] | [mL] |
| AMS 0.6 | 1X | 0.40 | 0.04 | 160 | 132 | 0.7 |
| AMS 0.6 | 8X(Gly) | 1.00 | 0.10 | 251 | 187 | 0.5 |

| | | | SPPS reactor |
|---|---|---|---|
| Theoretical | | | throughput |
| crude peptide | | | (mass after |
| mass after | Crude peptide | HPLC purity | cleavage/final |
| cleavage | after cleavage | after cleavage | volume) |
| [mg] | [mg] | [%] | [g/L] |
| 69 | 66 | 93 | 94.3 |
| 173 | 116 | 87 | 232.0 |

| Weight gain | Assembly yield | Cleavage yield |
|---|---|---|
| [mg] | [%] (1) | [%] (2) |
| 41 | 59 | 96 |
| 109 | 63 | 67 |

Application of support of invention significantly increases throughput while essentially preserving purity.

Synthesis Protocol for PP 6 (HAEGTFTSDVSSYLEGQAAKEFIAWLVRGRG) with DMF as Solvent

Starting resins (200 mg each) were introduced into 12 mL syringes and swollen in DMF (6.5 mL/g, 2×1 h) for initial swelling measurements prior to their transfer to Symphony X reactors. Fmoc groups were deprotected prior to each coupling step using 20% piperidine in DMF (1×10 min+1×20 min) at room temperature. All amino acid couplings were carried out using an equimolar ratio (1:1:1) of Fmoc amino acid (0.30M in DMF), Oxyma (0.90M in DMF) and N′N′-Diisopropylcarbodiimide (0.90M in DMF) at 5-fold molar excess compared to synthesis scale. Couplings were carried out without pre-activation for 1 h 30 min at room temperature.

After syntheses completion, resins were transferred again to the 12 mL syringes to measure their final swellings then washed with (iPrOH, 3×) and methyl tert-butyl ether (MTBE, 3×) prior to their drying under vacuum overnight.

Dried Boc-protected peptides were cleaved from resins using a cleavage cocktail containing TFA/H₂O/DTT/TIS (13 mL/g of peptidyl resin, 85/5/5/5 v/v/w/v) for 3 h at room temperature. The resin was filtered out and washed with TFA (3 mL/g of peptidyl resin). Peptides were precipitated with MTBE (10-folds the volume of TFA) at 0° C. The resulting suspensions were transferred to 50 ml conical centrifuge tubes and centrifuged at 2500 rcf for 10 min prior to supernatants' decantation. The crude solid products were washed again with cold MTBE (5×) and dried under vacuum overnight.
Molecular weight: 3383.73
Calculated mass: +3/3=1128.91; +4/4=846.93
Observed mass (AMS 0.6 4×(Gly)): +3/3=1129.01; +4/4=847.06
Solvent used for the synthesis of PP 6 on Symphony X: DMF
Mass of resin at beginning of synthesis after coupling of HMPB-Glycine linker: 200 mg.

All amino acids used were Fmoc-protected except for the last amino acid (His) which was Boc-protected.
Side chains protecting groups involved: tBu, Boc, Trt, Pbf: R=Arg(Pbf), W=Trp(Boc), E=Glu(OtBu), K=Lys(Boc), Q=Gln(Trt), Y=Tyr(tBu), S=Ser(tBu), D=Asp(OtBu), T=Thr(tBu), H=His(Trt)
Molecular weight of protected PP 6:5234.46 g/mol
Molecular weight of unprotected PP 6:3383.73 g/mol

TABLE 14
Data synthesis of PP 6 polypeptide: HAEGTFTSDVSSYLEGQAAKEFIAWLVRGRG

| | | | | Theoretical | Final mass | Final volume |
|---|---|---|---|---|---|---|
| | | Substitution | | mass of Boc | of Boc | of Boc |
| Unmodified | | at beginning | Synthesis | protected | protected | protected |
| matrix/resin | Type of | of synthesis | scale | peptidyl resin | peptidyl resin | peptidyl resin |
| [mmol/g] | construct | [mmol/g] | [mmol] | [mg] | [mg] | [mL] |
| AMS 0.93 | 1X | 0.53 | 0.106 | 731 | 479 | 1.7 |
| AMS 0.60 | 4X(Gly) | 0.88 | 1.176 | 1082 | 728 | 2.1 |

| | | | SPPS reactor |
|---|---|---|---|
| Theoretical | | | throughput |
| crude peptide | | | (mass after |
| mass after | Crude peptide | HPLC purity | cleavage/final |
| cleavage | after cleavage | after cleavage | volume) |
| [mg] | [mg] | [%] | [g/L] |
| 407 | 288 | 30 | 169.4 |
| 676 | 500 | 28 | 280.1 |

| Weight gain | Assembly yield | Cleavage yield |
|---|---|---|
| [mg] | [%] (1) | [%] (2) |
| 303 | 55 | 71 |
| 567 | 62 | 74 |

1. An insoluble support (resin) in particulate form comprising distal binding sites, the support comprising a homogeneous polymeric matrix and constructs, the constructs covalently bound to the polymeric matrix, wherein the constructs comprise at least one branching agent selected from aminoalkanoic acids comprising at least 2 amino groups and from 3 up to 10 carbon atoms, cleavable linkers and at least one spacer coupled to at least one branching agent via an amide bond, the cleavable linkers providing the distal binding sites.

2. The insoluble support according to claim 1, wherein the at least one branching agent is selected from aminoalkanoic acids comprising at least 2 but not more than 3 amino groups and from 3 up to 10 carbon atoms.

3. The insoluble support according to claim 1, wherein the at least one branching agent is selected from diaminoalkanoic acids comprising from 3 up to 10 carbon atoms.

4. The insoluble support according to claim 1, wherein the at least one branching agents are selected from diaminoalkanoic acids comprising from 3 up to 8 carbon atoms.

5. The insoluble support according to claim 1, wherein the at least one branching agent is selected from 2,3-diaminopropionic acid (Dpr), 2,4-diaminobutyric acid and 2,5-diaminopentanoic acid (ornithine), 2,6-diaminohexanoic acid, suitably the branching agent is selected from 2,4-diaminobutyric acid and 2,5-diaminopentanoic acid (ornithine), 2,6-diaminohexanoic acid.

6. The insoluble support according to claim 1, wherein the at least one branching agent is lysine.

7. The insoluble support according to claim 1, wherein all branching agents are identical.

8. The insoluble support according to claim 3, wherein the number of branching agents d of a construct is given by the formula d(n)=2ⁿ−1 and the number of linkers providing distal binding site 1 is given by the formula l(n)=2ⁿ, where n denotes the number of generations of branching agents and n being from 1 to 10.

9. The insoluble support according to claim 8, wherein n is from 2 to 10.

10. The insoluble support according to claim 9, wherein the at least one spacer is positioned between all branching agents.

11. The insoluble support according to claim 3, wherein the constructs are selected from:
(A) [polymeric matrix]-BA(1)-LK₂;
(B) [polymeric matrix]-BA(1)-BA(2)₂-LK₄;
(C) [polymeric matrix]-BA(1)-BA(2)₂-BA(3)₄-LK₈;
(D) [polymeric matrix]-BA(1)-BA(2)₂-BA(3)₄-BA(4)₈-LK₁₆;
(E) [polymeric matrix]-BA(1)-BA(2)₂-BA(3)₄-BA(4)₈-BA(5)₁₆-LK₃₂;
where BA denotes a branching agent and the integer in brackets indicates the generation of branching agent, and LK denotes cleavable linkers.

12. The insoluble support according to claim 1; wherein the at least one spacer molecule is selected from organic molecules comprising two binding sites.

13. The insoluble support according to claim 1, wherein the at least one spacer molecule is selected from organic molecules comprising two binding sites selected form any one of carboxylic acids, amines, hydroxyls.

14. The insoluble support according to claim 1; wherein the at least one spacer molecule is selected from organic molecules comprising two binding sites selected form amino acids and polyethylene glycol.

15. The insoluble support according to claim 1, wherein at least one spacer is selected from amino acids comprising two binding sites, suitably glycine and alanine.

16. The insoluble support according to claim 1, wherein the linkers are selected form Rink amide, Wang, 2-chlorotrityl, PAM, PAL, HMPB, Sieber and Ramage.

17. The insoluble support according to claim 1; wherein the polymeric matrix is selected from homogeneous polymeric matrices comprising primary binding sites distributed throughout the polymeric matrix.

18. The insoluble support according to claim 1, wherein the polymeric matrix is selected from homogeneous polymeric matrices formed by emulsion polymerization comprising at least styrene and divinylbenzene (DVB).

19. The insoluble support according to claim 1, wherein the polymeric matrix is selected from homogeneous polymeric matrices formed from a polymerization composition comprising at least styrene and divinylbenzene (DVB) and DVB being present in an amount of below 4.0 wt %.

20. (canceled)

21. (canceled)

22. A method for forming an insoluble support as defined by claim 1, the method comprising providing a polymeric matrix comprising primary binding sites, and wherein the construct is formed by a divergent synthesis approach, a convergent synthesis approach or a combined divergent and convergent synthesis approach.

23. A method for forming an insoluble support as defined by claim 1, the method comprising providing a polymeric matrix comprising primary binding sites, and wherein the constructs are formed by a method comprising at least the steps:
a) optionally coupling at least one spacer to the primary binding sites,
b) coupling a branching agent to the primary binding sites of the base matrix, or optionally coupling the branching agent to at least one spacer, where the at least two amino groups are protected by protecting groups,
c) removal of the protecting groups,
where steps a), b) and c) may be repeated, and
d) coupling of linkers to binding sites of distal branching agents.

24. A solid phase peptides synthesis protocol, solid phase morpholino oligomer synthesis and solid phase oligonucleotides synthesis for the synthesis of polypeptides, morpholino oligomers and oligonucleotides, the protocol comprising using an insoluble support as defined by claim 1.

25. A solid phase peptides synthesis protocol for the synthesis of polypeptides, the protocol comprising using an insoluble support as defined by claim 1.

26. The solid phase peptides synthesis protocol according to claim 25, wherein the polypeptides have at least 15 amino acids.
